SEQUENCE LISTING (31 sequences)

SEQ ID NO: 1 - protein, 632 aa, Trichoderma reesei
MHVLSTAVLLGSVAVQKVLGRPGSSGLSDVTKRSVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 2 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSGTPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 3 - protein, 453 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTC

SEQ ID NO: 4 - DNA, 1899 bp, Trichoderma reesei
atgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggaaccaaaccggctttgacctctgggaagaagtcaatgggagctcattctttactgttgccaaccagcaccgagcacttgtcgagggcgccactcttgctgccactcttggccagtcgggaagcgcttattcatctgttgctccccaggttttgtgctttctccaacgattctgggtgtcgtctggtggatacgtcgactccaacatcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcgcttcacctgacgtggtcgtacgcctcgttcttgacagccacggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccagacggtcaaggtggcgggcaacgccgcggccctgggcaactggagcacgagcgccgccgtggctctggacgccgtcaactatgccgataaccaccccctgtggattgggacggtcaacctcgaggctggagacgtcgtggagtacaagtacatcaatgtgggccaagatggctccgtgacctgggagagtgatcccaaccacacttacacggttcctgcggtggcttgtgtgacgcaggttgtcaaggaggacacctggcagtcgtaa

SEQ ID NO: 5 - protein, 448 aa, Aspergillus awamori
ATLDSWLSNEATVARTAILNNIGADGAWVSGADSGIVVASPSTDNPDYFYTWTRDSGLVIKTLVDLFRNGDTDLLSTIENYISSQAIVQGISNPSGDLSSGGLGEPKFNVDETAYTGSWGRPQRDGPALRATAMIGFRQWLLDNGYTSAATEIVWPLVRNDLSYVAQYWNQTGYDLWEEVNGSSFFTIAVQHRALVEGSAFATAVGSSCSWCDSQAPQILCYLQSFWTGEYILANFDSSRSGKDTNTLLGSIHTFDPEAGCDDSTFQPCSPRALANHKEVVDSFRSIYTLNDGLSDSEAVAVGRYPKDSYYNGNPWFLCTLAAAEQLYDALYQWDKQGSLEITDVSLDFFQALYSDAATGTYSSSSSTYSSIVDAVKTFADGFVSIVETHAASNGSLSEQYDKSDGDELSARDLTWSYAALLTANNRRNSVMPPSWGETSASSVPGTC

SEQ ID NO: 6 - protein, 449 aa, Aspergillus niger
ATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTC

SEQ ID NO: 7 - protein, 450 aa, Aspergillus oryzae
QSDLNAFIEAQTPIAKQGYLNNIGADGKLVEGAAAGIVYASPSKSNPDYFYTWTRDAGLTMEEYIEQFIGGDATLESTIQNYVDSQANEQAVSNPSGGLSDGSGLAEPKFYYNISQFTDSWGRPQRDGPALRASALIAYGNSLISSDKQSVVKANIWPIYQNDLSYVGQYWNQTGFDLWEEVQGSSFFTVAVQHKALVEGDAFAKALGEECQACSVAPQILCHLQDFWNGSAVLSNLPTNGRSGLDTNSLLGSIHTFDPAAACDDTTFQPCSSRALSNHKLVVDSFRSVYGINNGRGAGKAAAVGPYAEDTYQGGNPWYLTTLVAAELLYDALYQWDKQGQVNVTETSLPFFKDLSSNVTTGSYAKSSSAYESLTSAVKTYADGFISVVQEYTPDGGALAEQYSRDQGTPVSASDLTWSYAAFLSAVGRRNGTVPASWGSSTANAVPSQC

SEQ ID NO: 8 - protein, 441 aa, Humicola grisea
AAVDTFINTEKPIAWNKLLANIGPNGKAAPGAAAGVVIASPSRTDPPYFFTWTPDAALVLTGIIESLGHNYNTTLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECPPCTTVAPQVLCFQQAFWNSKGNYVVSTSTAGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTC

SEQ ID NO: 9 - protein, 452 aa, Hypocrea vinosa
SVDDFINTQTPIALNNLLCNVGPDGCRAFGTSAGAVIASPSTTDPDYYYMWTRDSALVFKNIVDRFTQQYDAGLQRRIEQYISAQVTLQGISNPSGSLSDGSGLGEPKFELTLSQFTGNWGRPQRDGPALRAIALIGYSKWLINNNYQSTVSNIIWPIVRNDLNYVAQYWNQTGFDLWEEVNGSSFFTVANQHRALVEGATLAATLGQSGSTYSSVAPQILCFLQRFWVSGGYIDSNINTNEGRTGKDANSLLASIHTFDPSLGCDASTFQPCSDKALSNLKVVVDSFRSIYGVNKGIPAGSAVAIGRYPEDVYFNGNPWYLATFAAAEQLYDSVYVWKKTGSITVTSTSSAFFQELVPGVAAGTYSSSQSTFTSIINAISTYADGFLSEAAKYVPADGSLAEQFDRNTGTPLSAVHLTWSYASFLTAAARRAGVVPPSWASSGANTVPSSC

SEQ ID NO: 10 - protein, 37 aa, Trichoderma reesei
SGASVVGSYSRPTATSFPPSQTPKPGVPSGTPYTPLP

SEQ ID NO: 11 - protein, 109 aa, Trichoderma reesei
CATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 12 - protein, 6 aa, Trichoderma reesei
SVDDFI

SEQ ID NO: 13 - protein, 599 aa, Trichoderma reesei
SVDDFISTETPIALNNLLCNVGPDGCRAFGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSAVHLTWSYASFLTAAARRAGIVPPSWANSSASTIPSTCSGASVVGSYSRPTATSFPPSQTPKPGVPSGTPYTPLPCATPTSVAVTFHELVSTQFGHTVKVAGNAAALGNWSTSAAVALDAVNYRDNHPLWIGTVNLEAGDVVEYKYIIVGQDGSVTWESDPNHTYTVPAVACVTQVVKEDTWQS

SEQ ID NO: 14 - protein, 599 aa, Trichoderma reesei
SVDDFISTETPIALNNLLCNVGPDGCRAVGTSAGAVIASPSTIDPDYYYMWTRDSALVFKNLIDRFTETYDAGLQRRIEQYITAQVTLQGLSNPSGSLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSAVHLTWSYASFLTAAARRAGIVPPSWANSSASTIPSTCSGASVVGSYSRPTATSFPPSQTPKPGVPSSTPYTPLPCATPTSVAVTFHELVSTQFGHTVKVAGNAAALGNWSTSAAVALDAVNYRDNHPLWIGTVNLEAGDVVEYKYIIVGQDGSVTWESDPNHTYTVPAVACVTQVVKEDTWQS

SEQ ID NO: 15 - protein, 599 aa, Trichoderma reesei
SVDDFISTETPIALNNLLCNVGPDGCRAFGTSAGAVIASPSTIDPDYVYMWTRDSALVFKNLIDRFTETYDAGLQRRIEQYITAQVTLQGLSNPSGSEADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSAVHLTWSYASFLTAAARRAGIVPPSWANSSASTIPSTCSGASVVGSYSRPTATSFPPSQTPKPGVPSGTPYTPLPCATPTSVAVTFSELVSTQFGHTVKVAGNAAALGNWSTSAAVALDAVNYRDNHPLWIGTVNLEAGDVVEYKYIIVGQDGSVTWESDPNHTYTVPAVACVTQVVKEDTWQS

SEQ ID NO: 16 - protein, 599 aa, Trichoderma reesei
SVDDFISTETPIALNNLLCNVGPDGCRAVGTSAGAVIASPSTIDPDYYYMWTRDSALVFKNLIDRFTETYDAGLQRRIEQYITAQVTLQGLSNPSGMLADGSGLGEPKFELTLKPFTGNWGRPQRDGPALRAIALIGYSKWLINNNYQSTVSNVIWPIVRNDLNYVAQYWNQTGFDLWEEVNGSSFFTVANQHRALVEGATLAATLGQSGSAYSSVAPQVLCFLQRFWVSSGGYVDSNINTNEGRTGKDVNSVLTSIHTFDPNLGCDAGTFQPCSDKALSNLKVVVDSFRSIYGVNKGIPAGAAVAIGRYAEDVYYNGNPWYLATFAAAEQLYDAIYVWKKTGSITVTATSLAFFQELVPGVTAGTYSSSSSTFTNIINAVSTYADGFLSEAAKYVPADGSLAEQFDRNSGTPLSALHLTWSYASFLTATARRAGIVPPSWANSSASTIPSTCSGASVVGSYSRPTATSFPPSQTPKPGVPSSWPYTPLPCATPTSVAVTFHELVSTQFGQTVKVAGNAAALGNWSTSAAVALDAVNYADNHPLWIGTVNLEAGDVVEYKYINVGQDGSVTWESDPNHTYTVPAVACVTQVVKEDTWQS

SEQ ID NO: 17 - protein, 599 aa, Trichoderma reesei; identical to SEQ ID NO: 13

SEQ ID NO: 18 - protein, 614 aa, Aspergillus awamori
TLDSWLSNEATVARTAILNNIGADGAWVSGADSGIVVASPSTDNPDYFYTWTRDSGLVIKTLVDLFRNGDTDLLSTIENYISSQAIVQGISNPSGDLSSGGLGEPKFNVDETAYTGSWGRPQRDGPALRATAMIGFRQWLLDNGYTSAATEIVWPLVRNDLSYVAQYWNQTGYDLWEEVNGSSFFTIAVQHRALVEGSAFATAVGSSCSWCDSQAPQILCYLQSFWTGEYILANFDSSRSGKDTNTLLGSIHTFDPEAGCDDSTFQPCSPRALANHKEVVDSFRSIYTLNDGLSDSEAVAVGRYPKDSYYNGNPWFLCTLAAAEQLYDALYQWDKQGSLEITDVSLDFFQALYSDAATGTYSSSSSTYSSIVDAVKTFADGFVSIVETHAASNGSLSEQYDKSDGDELSARDLTWSYAALLTANNRRNSVMPPSWGETSASSVPGTCAATSASGTYSSVTVTSSPSIVATGGTTTTATTTGFGGVTSTSKTTTTASKTSTTTSSTSCTTPTAVAVTFDLTATTTYGENIYLVGSISQLGDWDTSDGIALSADKYTSSNPLWYVTVTLPAGESFEYKFIRIESDDSVEWESDPNREYTVPQACGESTATVTDTWR

SEQ ID NO: 19 - protein, 616 aa, Aspergillus niger
ATLDSWLSNEATVARTAILNNIGADGAWVSGADSGIVVASPSTDNPDYFYTWTRDSGLVLKTLVDLFRNGDTSLLSTIENYISAQAIVQGISNPSGDLSSGAGLGEPKFNVDETAYTGSWGRPQRDGPALRATAMIGFGQWLLDNGYTSTATDIVWPLVRNDLSYVAQYWNQTGYDLWEEVNGSSFFTIAVQHRALVEGSAFATAVGSSCSWCDSQAPEILCYLQSFWTGSFILANFDSSRSGKDANTLLGSIHTFDPEAACDDSTFQPCSPRALANHKEVVDSFRSIYTLNDGLSDSEAVAVGRYPEDTYYNGNPWFLCTLAAAEQLYDALYQWDKQGSLEVTDVSLDFFKALYSDAATGTYSSSSSTYSSIVDAVKTFADGFVSIVETHAASNGSMSEQYDKSDGEQLSARDLTWSYAALLTANNRRNSVVPASWGETSASSVPGTCAATSAIGTYSSVTVTSWPSIVATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 20 - protein, 476 aa, Aspergillus oryzae
HPSFPIHKRQSDLNAFIEAQTPIAKQGVLNNIGADGKLVEGAAAGIVVASPSKSNPDYFYTWTRDAGLTMEEVIEQFIGGDATLESTIQNYVDSQANEQAVSNPSGGLSDGSGLAEPKFYVNISQFTDSWGRPQRDGPALRASALIAYGNSLISSDKQSVVKANIWPIVQNDLSYVGQYWNQTGFDLWEEVQGSSFFTVAVQHKALVEGDAFAKALGEECQACSVAPQILCHLQDFWNGSAVLSNLPTNGRSGLDTNSLLGSIHTFDPAAACDDTTFQPCSSRALSNHKLVVDSFRSVYGINNGRGAGKAAAVGPYAEDTYQGGNPWYLTTLVAAELLYDALYQWDKQGQVNVTETSLPFFKDLSSNVTTGSYAKSSSAYESLTSAVKTYADGFISVVQEYTPDGGALAEQYSRDQGTPVSASDLTWSYAAFLSAVGRRNGTVPASWGSSTANAVPSQCSGGTVSGSYTTPTVGSW

SEQ ID NO: 21 - protein, 601 aa, Humicola grisea
RPHGSSRLQERAAVDTFINTEKPIAWNKLLANIGPNGKAAPGAAAGVVIASPSRTDPPYFFTWTPDAALVLTGIIESLGHNYNTTLQQVSNPSGTFADGSGLGEAKFNVDLTAFTGEWGRPQRDGPPLRAIALIQYAKWLIANGYKSTAKSVVWPVVKNDLAYTAQYWNETGFDLWEEVPGSSFFTIASSHRALTEGAYLAAQLDTECPPCTTVAPQVLCFQQAFWNSKGNYVVSTSTAGEYRSGKDANSILASIHNFDPEAGCDNLTFQPCSERALANHKAYVDSFRNLYAINKGIAQGKAVAVGRYSEDVYYNGNPWYLANFAAAEQLYDAIYVWNKQGSITVTSVSLPFFRDLVSSVSTGTYSKSSSTFTNIVNAVKAYADGFIEVAAKYTPSNGALAEQYDRNTGKPDSAADLTWSYSAFLSAIDRRAGLVPPSWRASVAKSQLPSTCSRIEVAGTYVAATSTSFPSKQTPNPSAAPSPSPYPTACADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 22 - protein, 611 aa, Hypocrea vinosa
RPGSNGLSDITKRSVDSFISAETPIALNNLLCNVGPDGCRAFGTSAGAVIASPSTVDPDYYYMWTRDSALVFKNIVDRFTQKYDAGLQRRIEQYISAQVTLQGISNPSGSLSDGSGLGEPKFELTLNQFTGNWGRPQRDGPALRAIALIGYSKWLINNNYQSTVSSVIWPIVKNDLNYVAQYWNQTGFDLWEEVNGSSFFTVANQHRALVEGATLATTLGQSGSTYSSVAPQILCFLQRFWVSGSYIDSNINVNEGRTGKDANSLLASIHTFDPSLGCDASTFQPCSDKALSNLKVVVDSFRSIYGVNSGISASSAVAIGRYPEDVYFNGNPWYLATFAAAEQLYDALYVWKQAGSITVTSTSLAFFQQLVPGVAAGTYSSSQSTYTSIINAVSAYADGFMNEAAKYVPADGSLAEQFDKNSGTPLSAVHLTWSYASFLTAADRRAGIVPSSWASSGANTVPSSCSGASVVGSYSRPTATSFPPSQTPKPGVPSGTPFTPIPCATPTSVAVTFHELATTQFGQTIKVVGSVPELGNWSTNAAVALNAVNYASNHPLWLGSINLAAGEVVQYKYINVGSDGSVTWESDPNHTYTVPAVACVTQVVKEDTWQS

SEQ ID NO: 23 - protein, 588 aa, Talaromyces sp.
GSLDSFLATETPIALQGVLNNIGPNGADVAGASAGIVVASPSRSDPDYFYSWTRDAALTAKYLVDAFIAGNKDLEQTIQEYISAQAQVQTISNPSGDLSTGGLGEPKFNVNETAFTGPWGRPQRDGPALRATALIAYANYLIDNGQASTADEIIWPIVQNDLSYVTQYWNSSTFDLWEEVEGSSFFTTAVQHRALVEGNALATRLNHTCPNCVSQAPQVLCFLQSYWTGSYVLANFGGSGRSGKDVNSILGSIHTFDPAGGCDDSTFQPCSARALANHKVVTDSFRSVYAVNSGIAEGSAVAVGRYPEDVYQGGNPWYLATAAAAEQLYDAIYQWNKIGSISITDVSLAFFQDIYPSAAVGTYNSGSSTFNDIISAVQTYADGYLSIIEKYTPSDGSLTEQFSRSDGTPLSASGLTWSYASLLTAAARRQSIVPASWGESSASSVPAVCSATSATGPYSTATNTAWPSSGSGPSTTTSVPCTTPTSVAVTFDEIVSTTYGETIYLAGSIPELGNWSPSSAIPLRADAYTSSNPLWYVTLNLPAGTSFEYKFFKKETDGTIVWEDDPNRSYTVPAYCGQTTAILDDSWQ

SEQ ID NO: 24 - protein, 112 aa, Humicola grisea
CADASEVYVTFNERVSTAWGETIKVVGNVPALGNWDTSKAVTLSASGYKSNDPLWSITVPIKATGSAVQYKYIKVGTNGKITWESDPNRSITLQTASSAGKCAAQTVNDSWR

SEQ ID NO: 25 - protein, 109 aa, Thermomyces lanuginosus
CTPPSEVTLTFNALVDTAFGQNIYLVGSIPELGSWDPANALLMSAKSWTSGNPVWTLSISLPAGTSFEYKFIRKDDGSSDVVWESDPNRSYNVPKDCGANTATVNSWWR

SEQ ID NO: 26 - protein, 108 aa, Talaromyces emersonii
CTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 27 - protein, 108 aa, Aspergillus niger [sequence omitted]

SEQ ID NO: 28 - protein, 108 aa, Aspergillus awamori
CTTPTAVAVTFDLTATTTYGENIYLVGSISQLGDWDTSDGIALSADKYTSSNPLWYVTVTLPAGESFEYKFIRIESDDSVEWESDPNREYTVPQACGESTATVTDTWR

SEQ ID NO: 29 - protein, 108 aa, Thielavia terrestris
CSTPTAVAVTFNERVTTQWGQTIKVVGDAAALGGWDTSKAVPLSAAGYTASDPLWSGTVDLPAGLAVQYKYINVAADGGVTWEADPNHSFTVPAACGTTAVTRDDTWQ

SEQ ID NO: 30 - DNA, 1899 bp, Trichoderma reesei; identical to SEQ ID NO: 4

SEQ ID NO: 31 - DNA, 1899 bp, Trichoderma reesei
atgcacgtcctgtcgactgcggtgctgctcggctccgttgccgttcaaaaggtcctgggaagaccaggatcaagcggtctgtccgacgtcaccaagaggtctgttgacgacttcatcagcaccgagacgcctattgcactgaacaatcttctttgcaatgttggtcctgatggatgccgtgcattcggcacatcagctggtgcggtgattgcatctcccagcacaattgacccggactactattacatgtggacgcgagatagcgctcttgtcttcaagaacctcatcgaccgcttcaccgaaacgtacgatgcgggcctgcagcgccgcatcgagcagtacattactgcccaggtcactctccagggcctctctaacccctcgggctccctcgcggacggctctggtctcggcgagcccaagtttgagttgaccctgaagcctttcaccggcaactggggtcgaccgcagcgggatggcccagctctgcgagccattgccttgattggatactcaaagtggctcatcaacaacaactatcagtcgactgtgtccaacgtcatctggcctattgtgcgcaacgacctcaactatgttgcccagtactggaaccaaaccggctttgacctctgggaagaagtcaatgggagctcattctttactgttgccaaccagcaccgagcacttgtcgagggcgccactcttgctgccactcttggccagtcgggaagcgcttattcatctgttgctccccaggttttgtgctttctccaacgattctgggtgtcgtctggtggatacgtcgactccaacatcaacaccaacgagggcaggactggcaaggatgtcaactccgtcctgacttccatccacaccttcgatcccaaccttggctgtgacgcaggcaccttccagccatgcagtgacaaagcgctctccaacctcaaggttgttgtcgactccttccgctccatctacggcgtgaacaagggcattcctgccggtgctgccgtcgccattggccggtatgcagaggatgtgtactacaacggcaacccttggtatcttgctacatttgctgctgccgagcagctgtacgatgccatctacgtctggaagaagacgggctccatcacggtgaccgccacctccctggccttcttccaggagcttgttcctggcgtgacggccgggacctactccagcagctcttcgacctttaccaacatcatcaacgccgtctcgacatacgccgatggcttcctcagcgaggctgccaagtacgtccccgccgacggttcgctggccgagcagtttgaccgcaacagcggcactccgctgtctgcggttcacctgacgtggtcgtacgcctcgttcttgacagccgcggcccgtcgggctggcatcgtgcccccctcgtgggccaacagcagcgctagcacgatcccctcgacgtgctccggcgcgtccgtggtcggatcctactcgcgtcccaccgccacgtcattccctccgtcgcagacgcccaagcctggcgtgccttccggtactccctacacgcccctgccctgcgcgaccccaacctccgtggccgtcaccttccacgagctcgtgtcgacacagtttggccatacggtcaaggtggcgggcaacgccgcggccctgggcaactggagcacgagcgccgccgtggctctggacgccgtcaactatcgtgataaccaccccctgtggattgggacggtcaacctcgaggctggagacgtcgtggagtacaagtacatcattgtgggccaagatggctccgtgacctgggagagtgatcccaaccacacttacacggttcctgcggtggcttgtgtgacgcaggttgtcaaggaggacacctggcagtcgtaa

(19) United States
(12) Patent Application Publication
(10) Pub. No.: US 2017/0306309 A1
(43) Pub. Date: Oct. 26, 2017

(54) VARIANTS HAVING GLUCOAMYLASE ACTIVITY

(71) Applicant: DuPont Nutrition Biosciences APS, Copenhagen (DK)

(72) Inventors: Jacob Flyvholm CRAMER, Højbjerg (DK); Peter Edvard DEGN, Egå (DK); Igor NIKOLAEV, Noordwijk (NL); Viktor ALEKSEYEV, Palo Alto, CA (US); Casper Willem VROEMEN, Palo Alto, CA (US); Daniel TORRES PAZMINO, Overrijn 12 (NL); Neville Marshall FISH, Stockport (GB)

(73) Assignee: DuPont Nutrition Biosciences APS, Copenhagen (DK)

(21) Appl. No.: 15/485,509

(22) Filed: Apr. 12, 2017

Related U.S. Application Data
(63) Continuation of application No. 14/423,063, filed on Feb. 20, 2015, now abandoned.
(60) Provisional application No. 61/692,040, filed on Aug. 22, 2012.

(30) Foreign Application Priority Data
Oct. 23, 2012 (EP) 12189588.2

(51) Int. Cl. C12N 9/34; C12C 11/00; C12C 7/04

(52) U.S. Cl. CPC C12N 9/2428; C12Y 302/01003; C12C 7/04; C12C 11/00; Y02E 10/38

(57) ABSTRACT

The present invention relates to variants having glucoamylase activity with improved properties and to compositions comprising these variants suitable for use for example in the production of a food, beverage (e.g. beer), feed, biochemical, or biofuel. Also disclosed are DNA constructs encoding the variants and methods of producing the glucoamylase variants in host cells. Furthermore, different methods and uses related to glucoamylases according to the invention are disclosed, such as in a brewing process.

### REFERENCE TO A SEQUENCE LISTING

Attached is a sequence listing comprising SEQ ID NOs: 1-31, which are herein incorporated by reference in their entirety.

### FIELD OF THE INVENTION

The present invention relates to variants having glucoamylase activity with improved properties and to compositions comprising these variants suitable for use for example in the production of a food, beverage (e.g. beer), feed, biochemical, or biofuel. Also disclosed are DNA constructs encoding the variants and methods of producing the glucoamylase variants in host cells. Furthermore, different methods and uses related to glucoamylases according to the invention are disclosed, such as in a brewing process.

### BACKGROUND

Glucoamylases (glucan 1,4-α-glucohydrolases, EC 3.2.1.3) are starch hydrolyzing exo-acting carbohydrases, which catalyze the removal of successive glucose units from the non-reducing ends of starch or related oligo and polysaccharide molecules. Glucoamylases can hydrolyze both the linear and branched glucosidic linkages of starch (e.g., amylose and amylopectin).

Glucoamylases are produced by numerous strains of bacteria, fungi, yeast and plants. Particularly interesting, and commercially important, glucoamylases are fungal enzymes that are extracellularly produced, for example from strains of Aspergillus (Svensson et al., Carlsberg Res. Commun. 48: 529-544 (1983); Boel et al., EMBO J. 3: 1097-1102 (1984); Hayashida et al., Agric. Biol. Chem. 53: 923-929 (1989); U.S. Pat. No. 5,024,941; U.S. Pat. No. 4,794,175 and WO 88/09795); Talaromyces (U.S. Pat. No. 4,247,637; U.S. Pat. No. 6,255,084; and U.S. Pat. No. 6,620,924); Rhizopus (Ashikari et al., Agric. Biol. Chem. 50: 957-964 (1986); Ashikari et al., App. Microbio. Biotech. 32: 129-133 (1989) and U.S. Pat. No. 4,863,864); Humicola (WO 05/052148 and U.S. Pat. No. 4,618,579); and Mucor (Houghton-Larsen et al., Appl. Microbiol. Biotechnol. 62: 210-217 (2003)). Many of the genes that code for these enzymes have been cloned and expressed in yeast, fungal and/or bacterial cells. Commercially, glucoamylases are very important enzymes and have been used in a wide variety of applications that require the hydrolysis of starch (e.g., for producing glucose and other monosaccharides from starch). Glucoamylases are used to produce high fructose corn sweeteners, which comprise over 50% of the sweetener market in the United States.

In general, glucoamylases may be, and commonly are, used with alpha-amylases in starch hydrolyzing processes to hydrolyze starch to dextrins and then glucose. The glucose may then be converted to fructose by other enzymes (e.g., glucose isomerases); crystallized; or used in fermentations to produce numerous end products (e.g., ethanol, citric acid, lactic acid, succinate, ascorbic acid intermediates, glutamic acid, glycerol and 1, 3-propanediol). Ethanol produced by using glucoamylases in the fermentation of starch and/or cellulose containing material may be used as a source of fuel or for alcoholic consumption.

At the high solids concentrations used commercially for high glucose corn syrup (HGCS) and high fructose corn syrup (HFCS) production, glucoamylase synthesizes di-, tri-, and tetra-saccharides from glucose by condensation reactions. This occurs because of the slow hydrolysis of alpha-(1-6)-D-glucosidic bonds in starch and the formation of various accumulating condensation products, mainly isomaltose, from D-glucose. Accordingly, the glucose yield in many conventional processes does not exceed 95% of theoretical yield. The amount of syrups produced worldwide by this process is very large and even very small increases in the glucose yield pr ton of starch are commercially important.

Several glucoamylases are described in for example WO/2008/045489, WO/2009/048488, WO/2009/048487, U.S. Pat. No. 8,058,033, WO/2011/022465, WO2011/020852 and WO 2012/001139.

The use of glucoamylases in the hydrolysis of starch derived carbohydrate has increasing importance in the brewing industry, particularly for the production of highly attenuated (sometimes referred to as low calorie) beers. Glucose is readily converted to alcohol by yeast making it possible for the breweries to obtain a very high alcohol yield from fermentation and at the same time obtain a beer, which is very low in residual carbohydrate. The ferment is diluted down to the desired alcohol % with water, and the final beer is sold as “low carb”. For reasons relating to product stability and legislation it is important that the added enzymatic activity is removed/inactivated in the final beer. Unfortunately this requirement is difficult to fulfill due to the thermostability of the enzymes, when the glucoamylase is derived from the usual source Aspergillus spp., such as A. niger and A. awamori; Humicola spp.; Talaromyces spp., such as T. emersonii; Athelia spp., such as A. rolfsii; Penicillium spp., such as P. chrysogenum, for example, and the enzyme is added into the fermenting vessel (FV) in the brewing process.

Although the addition of glucoamylase to the mashing vessel, or at any stage prior to wort boiling, may avoid this problem, this introduces other practical difficulties. U.S. Pat. No. 4,666,718 describes a brewing process employing a reactor comprising the brewing enzyme glucoamylase immobilised on a solid support, whereby the enzyme can be recovered from the product. U.S. Pat. No. 5,422,267A describes a brewing process employing genetically engineered yeast expressing a recombinant glucoamylase, but where the enzyme is secreted by the yeast.

Therefore, a need still exists for glucoamylases for example in the form of a composition having glucoamylase activity that can be added to any stage of a conventional process for preparing a fermented beverage such as beer using conventional equipment and whose activity can safely be removed from the final product.

It would be especially efficient to add glucoamylase variants having hydrolytic activity for example in the form of a composition into a fermentation vessel (FV) used in preparing a fermented beverage. The benefits are for example lower enzyme doses, increased starch conversion to fermentable carbohydrate and reduced yeast stress. The reason why this approach is not commonly used is that active enzymes then may be present in the final product, which is undesirable as described above. The commercially available glucoamylases are in general thermostable and the energy applied during pasteurisation of a fermented beverage is not sufficient to inactivate the enzymes. Thus, a further need exist for a thermolabile glucoamylase that may be inactivated by pasteurisation after fermentation.

### SUMMARY

The present invention relates to a glucoamylase variant comprising one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase.

The present invention also relates to a nucleic acid capable of encoding a glucoamylase variant of the present invention.

The present invention also relates to a nucleic acid capable of expressing a glucoamylase variant of the present invention. The present invention further relates to a plasmid or an expression vector such as a recombinant expression vector comprising the nucleic acid or capable of expressing a glucoamylase variant of the present invention. The present invention also relates to a host cell having heterologous expression of a glucoamylase variant of the present invention and a host cell comprising a plasmid or expressing vector as defined above.

The present invention further relates to methods of isolating, producing and/or expressing a glucoamylase variant of the present invention.

The present invention also relates to a composition comprising one or more glucoamylase variant (s) of the present invention.

The present invention also relates to the use of a glucoamylase variant or a composition of the present invention in a fermentation, wherein said glucoamylase variant or composition is added before or during a fermentation step.

The present invention also relates to the use of a thermolabile glucoamylase variant of the present invention to enhance the production of fermentable sugars in the fermentation step of a brewing process.

The present invention also relates to method which comprises adding a glucoamylase variant or a composition of the invention before or during a fermentation step.

The present invention also relates to a fermented beverage wherein the fermented beverage is produced by a method of the present invention.

The present invention also relates to a method for the production of a food, feed, or beverage product, such as an alcoholic or non-alcoholic beverage, such as a cereal- or malt-based beverage like beer or whiskey, such as wine, cider, vinegar, rice wine, soya sauce, or juice, said method comprising the step of treating a starch and/or sugar containing plant material with a glucoamylase variant or a composition of the present invention.

The present invention also relates to a method for the production of a first- or second-generation biofuel, such as bioethanol, said method comprising the step of treating a starch comprising material with a glucoamylase variant as described herein, and products obtained by such method. The present invention also relates to a method for the production of a biochemical, such as bio-based isoprene, said method comprising the step of treating a starch comprising material with a glucoamylase variant as described herein, and products obtained by such method. The present invention further relates to the use of a glucoamylase variant or a composition as disclosed herein in the production of a first- or second-generation biofuel, such as bioethanol, or in the production of a biochemical, such as bio-based isoprene.

The present invention also relates to a kit comprising a glucoamylase variant, or a composition of the present invention; and instructions for use of said glucoamylase variant or composition.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or embodiments, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following detailed description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 is a schematic representation of the entry vectors: A) pEntry-GA CS4 and B) pEntry-GA wt.

FIG. 2 is a schematic representation of the expression vectors: A) pTTT-pyrG13-GACS4 and B) pTTTpyr2-GACS4.

FIG. 3 depicts a SDS-PAGE analysis of TrGA variants. In top, Trichoderma reesei glucoamylase variants: R_C_1, R_C_2, R_C_5, R_C_12, R_C_7 R_D_2, R_D_3 and R_D_5; in bottom, variant R_C_13, R_C_22, R_A_1, R_A_2, R_A_6, R_A_7 and TrGA (wt). For each variant is the glucoamylase activity (GAU/mL) shown in brackets.

FIG. 4 depicts a SDS-PAGE analysis of TrGA variants. In top, Trichoderma reesei glucoamylase variants CPS3-B01-CPS2-E08 as indicated and the fermentation product of the empty vector and TrGA-CS4. For each variant is the glucoamylase activity (GAU/mL) shown in brackets.

FIG. 5 depicts a SDS-PAGE analysis of purified TrGA variants. From left: molecular weight marker and purified Trichoderma reesei glucoamylase variants R_C_1 and R_C_2 as indicated.

FIG. 6A depicts a comparison of the three dimensional structure of Trichoderma reesei glucoamylase (black) (SEQ ID NO: 2) and Aspergillus awamori glucoamylase (grey) (SEQ ID NO: 5) viewed from the side. The side is measured in reference to the active site and the active site entrance is at the ‘top’ of the molecule.

FIG. 6B depicts the three dimensional structure of Trichoderma reesei glucoamylase (black) (SEQ ID NO: 2) viewed from the side. The side is measured in reference to the active site and the active site entrance is at the “top” of the molecule. Residues forming the interface region in between the catalytic domain and the starch binding domain are shown as transparent spheres (residues from the catalytic domain are colored in dark gray and residues from the starch binding domain are colored in light gray).

FIG. 7 depicts a comparison of the three dimensional structures of Trichoderma reesei glucoamylase (black) (SEQ ID NO: 2) and Aspergillus awamori glucoamylase (grey) (SEQ ID NO: 5) viewed from the top.

FIG. 8 depicts an alignment of the three dimensional structures of TrGA (SEQ ID NO: 2) and AnGA (SEQ ID NO: 6) viewed from the side showing binding sites 1 and 2.

FIG. 9 depicts a model of the binding of acarbose in the TrGA structure.

FIGS. 10A and 10B depict an alignment comparison of the catalytic domains of parent glucoamylases from Aspergillus awamori (AaGA) (SEQ ID NO: 5); Aspergillus niger (AnGA) (SEQ ID NO: 6); Aspergillus oryzae (AoGA) (SEQ ID NO: 7); Trichoderma reesei (TrGA) (SEQ ID NO: 3); Humicola grisea (HgGA) (SEQ ID NO: 8); and Hypocrea vinosa (HvGA) (SEQ ID NO: 9). Identical amino acids are indicated by an asterisk (*).

FIG. 10C depicts a Talaromyces glucoamylase (TeGA) mature protein sequence (SEQ ID NO: 23).

FIGS. 10D and 10E depict an alignment comparing the Starch Binding Domain (SBD) of parent glucoamylases from Trichoderma reesei (SEQ ID NO: 11); Humicola grisea (HgGA) (SEQ ID NO: 24); Thermomyces lanuginosus (ThGA) (SEQ ID NO: 25); Talaromyces emersonii (TeGA) (SEQ ID NO: 26); Aspergillus niger (AnGA) (SEQ ID NO: 27); Aspergillus awamori (AaGA) (SEQ ID NO: 28); and Thielavia terrestris (TtGA) (SEQ ID NO: 29).

### SEQUENCES

Following the example section are sequences, which are herein incorporated by reference in their entirety.

SEQ ID NO: 1: Trichoderma reesei glucoamylase, full-length; with signal peptide
SEQ ID NO: 2: Trichoderma reesei glucoamylase, mature protein; without signal peptide
SEQ ID NO: 3: Trichoderma reesei glucoamylase catalytic domain, 1-453 of mature TrGA, CD
SEQ ID NO: 4: Trichoderma reesei glucoamylase cDNA
SEQ ID NO: 5: Aspergillus awamori GA (AaGA); CD
SEQ ID NO: 6: Aspergillus niger (AnGA), CD
SEQ ID NO: 7: Aspergillus oryzae (AoGA), CD
SEQ ID NO: 8: Humicola grisea glucoamylase (HgGA); CD
SEQ ID NO: 9: Hypocrea vinosa glucoamylase (HvGA); CD
SEQ ID NO: 10: TrGA, linker region

### SEQ ID NO: 11: TrGA, SBD

SEQ ID NO: 12: SVDDFI: start of the TrGA mature protein
SEQ ID NO: 13: Trichoderma reesei glucoamylase CS4 variant, mature protein; without signal peptide
SEQ ID NO: 14: Trichoderma reesei glucoamylase R_A_1 variant, mature protein; without signal peptide
SEQ ID NO: 15: Trichoderma reesei glucoamylase R_C_1 variant, mature protein; without signal peptide
SEQ ID NO: 16: Trichoderma reesei glucoamylase R_A_6 variant, mature protein; without signal peptide
SEQ ID NO: 17: Trichoderma reesei glucoamylase R_C_13 variant, mature protein; without signal peptide
SEQ ID NO: 18: Aspergillus awamori glucoamylase (AaGA), full-length, with signal peptide
SEQ ID NO: 19: Aspergillus niger glucoamylase (AnGA), full-length, with signal peptide
SEQ ID NO: 20: Aspergillus oryzae glucoamylase (AoGA), full-length, with signal peptide
SEQ ID NO: 21: Humicola grisea glucoamylase (HgGA), full-length, with signal peptide
SEQ ID NO: 22: Hypocrea vinosa glucoamylase (HvGA), full-length, with signal peptide
SEQ ID NO: 23: Talaromyces GA, mature protein
SEQ ID NO: 24: Humicola grisea GA, SBD
SEQ ID NO: 25: Thermomyces lanuginosus GA, SBD
SEQ ID NO: 26: Talaromyces emersonii GA, SBD
SEQ ID NO: 27: Aspergillus niger GA, SBD
SEQ ID NO: 28: Aspergillus awamori GA, SBD
SEQ ID NO: 29: Thielavia terrestris GA, SBD
SEQ ID NO: 30: Trichoderma reesei wt glucoamylase optimized cDNA (pEntry-GA WT)
SEQ ID NO: 31: Trichoderma reesei CS4 variant glucoamylase optimized cDNA (pEntry-GA CS4)

### DETAILED DESCRIPTION

Glucoamylases are commercially important enzymes in a wide variety of applications that require the hydrolysis of starch. The applicants have found that by introducing certain alterations in positions within specific regions of the amino acid sequence of a parent glucoamylase the glucoamylase variant exhibit decreased thermostability and in some embodiments without loosing saccharification performance as compared to the parent glucoamylase.

### DESCRIPTION OF THE INVENTION

Glucoamylases are commercially important enzymes in a wide variety of applications that require the hydrolysis of starch. Disclosed herein are glucoamylase variants with reduced thermo stability for hydrolysis of starch. These glucoamylase variants contain amino acid substitutions within the catalytic domains and/or the starch binding domain. The variants display altered properties, such as an altered thermo stability and/or altered specific activity.

Furthermore, it is described herein that a certain subset of glucoamylase variants are very useful for addition into a fermentation vessel during for example beer fermentation because of the suitable thermolability of the enzyme which makes inactivation by pasteurisation possible.

Pasteurisation experiments have been performed on beer in lab-, pilot- and full-scale to assess the ability to inactivate the variants described herein in the brewing process. Lab-scale pasteurisations were validated on bottled beer with glucoamylases in a full-scale tunnel pasteuriser (data not shown). The present inventors have provided a number of variants of a parent glucoamylase, which variants in some embodiments have both shown to be functional active in the fermentation vessel (high saccharification performance) and significant more thermolabile than parent glucoamylase and/or several other tested glucoamylases. These glucoamylase variants may be completely inactivated with less than 16.8 pasteurisation units (PU), which is preferred for beer pasteurisation.

In some embodiments using a glucoamylase variant as described herein in a saccharification process produces a syrup with high glucose percentage. In some embodiments using a glucoamylase variant as described herein results in an enhanced production of fermentable sugars in a mashing and/or fermentation step of a brewing step. In some embodiments using a glucoamylase variant as described herein results in an enhanced real degree of fermentation. These altered properties are obtained by mutating e.g. substituting amino acid residues at selected positions in a parent glucoamylase. This will be described in more detail below.

In one aspect, described herein is glucoamylase variants comprising one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase.

In one aspect, described herein is glucoamylase variants comprising

a) an amino acid substitution at the residue corresponding to position 502 of SEQ ID NO: 2, or an equivalent position in a parent glucoamylase, and optionally an amino acid substitution selected from the group of interface amino acids consisting of residues 29, 43, 48, and 116 of SEQ ID NO: 2, or an equivalent position in a parent glucoamylase;
b) an amino acid substitution at the residue corresponding to position 98 of SEQ ID NO: 2, or an equivalent position in a parent glucoamylase, and optionally one or two amino acid substitutions selected from the group of catalytic core amino acid residues consisting of residues 97, 147, 175, 483 and 484 of SEQ ID NO: 2, or an equivalent position in a parent glucoamylase;
which glucoamylase variant at least has one amino acid substitution selected from said group of interface amino acids or said group of catalytic core amino acid residues;
wherein the glucoamylase variant has at least 80% sequence identity with SEQ ID NO: 1, 2, 13, 18, 19, 20, 21, or 22.

In one aspect, described herein is glucoamylase variants comprising

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase, or an amino acid substitution at the residue corresponding to position 147 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

In one aspect, described herein is glucoamylase variants comprising

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 147 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

In one aspect, described herein is glucoamylase variants

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

In one aspect, described herein is glucoamylase variants comprising the amino acid substitution H502S of SEQ ID NO: 2 or 13; the amino acid substitution L98E of SEQ ID NO: 2 or 13; and the amino acid substitution Y48V of SEQ ID NO: 2 or 13 or the amino acid substitution Y147R of SEQ ID NO: 2 or 13; wherein the glucoamylase variant has at least 80% sequence identity with SEQ ID NO: 2 or 13.

In one aspect, described herein is glucoamylase variants with a starch binding domain and a catalytic domain, said variant comprising one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase.

In one aspect, described herein is glucoamylase variants further comprising one or two amino acid substitutions in the group of interface amino acids consisting of residues 24, 26, 27, 30, 40, 42, 44, 46, 49, 110, 111, 112, 114, 117, 118, 119, 500, 504, 534, 536, 537, 539, 541, 542, 543, 544, 546, 547, 548, 580, 583, 585, 587, 588, 589, 590, 591, 592, 594, and 596 of SEQ ID NO:2 or an equivalent position in a parent glucoamylase.

In a further aspect, described herein is glucoamylase variants further comprising one, two or three amino acid substitutions in the group of catalytic core amino acids consisting of residues in positions 1 to 484 with exception of position 24, 26, 27, 29, 30, 40, 42, 43, 44, 46, 48, 49, 97, 98, 110, 111, 112, 114, 116, 117, 118, 119, 147, 175, 483 and 484 of SEQ ID NO: 2, or an equivalent position in a parent glucoamylase.

In one aspect, described herein is glucoamylase variants comprising one or two amino acid substitutions in the group of interface amino acids consisting of residues 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596 of SEQ ID NO:2 or an equivalent position in a parent glucoamylase.

In a further aspect, described herein is glucoamylase variants comprising one, two or three amino acid substitutions in the group of catalytic core amino acids consisting of residues in positions 1 to 484 with exception of position 24, 26, 27, 29, 30, 40, 42, 43, 44, 46, 48, 49, 110, 111, 112, 114, 116, 117, 118 and 119 of SEQ ID NO: 2, or an equivalent position in a parent glucoamylase.

In one aspect, described herein is glucoamylase variants having an RDF of at least 74.5%, such as for example at least 75%, 76%, 77%, 78%, 79%, 80%, 81%, 82%, 83%, 84%, 85% 86%, 87%, 88%, 89% or 90% when dosed at 0.058 mg GA/ml wort as described the ‘Brewing’ analysis in the Assays and Methods section.

In one aspect, the present invention describes the structural-functional linkage used to derive a set of TrGA variants that is sufficiently thermolabile in beer to be completely inactivated by pasteurisation and at the same time maintain high performance throughout the beer fermentation evaluated by the real degree of fermentation. In a further aspect, the glucoamylase variant described herein comprises one or two amino acid substitutions in the group of interface amino acids consisting of residues F29, I43, Y48, F116 and H502 of SEQ ID NO: 2, wherein the substitution in I43 is I43Q, and the substitution in Y48 is Y48V, or an equivalent position in a parent glucoamylase; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W, or an equivalent position in a parent glucoamylase.

In one aspect, the parent glucoamylase as described herein is SEQ ID NO: 1, 2, 13, 18, 19, 20, 21, or 22. In a further aspect, the glucoamylase variant described herein has at least 80% sequence identity such as at least 85%, 90%, 95%, or 99.5% sequence identity with SEQ ID NO: 1, 2, 13, 18, 19, 20, 21, or 22. In one aspect, the parent glucoamylase described herein has a catalytic domain that has at least 80%, 85%, 90%, 95%, or 99.5% sequence identity with SEQ ID NO: 1, 2, 3, 5, 6, 7, 8, 9, and/or 13, and/or a starch binding domain that has at least 80%, 85%, 90%, 95%, or 99.5% sequence identity with SEQ ID NO: 11, 24, 25, 26, 27, 28, and/or 29.

In a further aspect, the glucoamylase variant described herein consist of the parent sequence of the amino acids of SEQ ID NO: 1, 2, 13, 18, 19, 20, 21, or 22, which sequence of amino acids has one or two amino acid substitutions in the group of interface amino acids consisting of residues F29, I43, Y48, F116 and H502 of SEQ ID NO: 2, wherein the substitution in I43 is I43Q, and the substitution in Y48 is Y48V, or an equivalent position in the parent glucoamylase; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W, or an equivalent position in the parent glucoamylase.

In a further aspect, the glucoamylase variant described herein consist of the sequence of the amino acids of SEQ ID NO: 2, which sequence of amino acids has one or two amino acid substitutions in the group of interface amino acids consisting of residues F29, I43, Y48, F116 and H502 of SEQ ID NO: 2, wherein the substitution in I43 is I43Q, and the substitution in Y48 is Y48V; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W.

In a further aspect, the glucoamylase variant described herein consist of the sequence of the amino acids of SEQ ID NO: 13, which sequence of amino acids has one or two amino acid substitutions in the group of interface amino acids consisting of residues F29, I43, Y48, F116 and H502 of SEQ ID NO: 13, wherein the substitution in I43 is I43Q, and the substitution in Y48 is Y48V; and one, two or three amino acid substitutions in the group of catalytic core amino acid residues consisting of residues S97, L98, Y147, F175, G483 and T484 of SEQ ID NO: 13, wherein the substitution in S97 is S97M, the substitution in G483 is G483S and the substitution in T484 is T484W SEQ ID NO: 13.

In one aspect, the glucoamylase variant exhibits altered thermostability as compared to the parent glucoamylase. In one aspect, the glucoamylase variant described herein exhibits decreased thermostability as compared to the parent glucoamylase, such as the parent glucoamylase to which it has the highest sequence identity to. In one aspect, the glucoamylase variant exhibits altered specific activity as compared to the parent glucoamylase, such as the parent glucoamylase to which it has the highest sequence identity to. In one aspect, the glucoamylase variant exhibits similar or increased specific activity as compared to the parent glucoamylase, such as the parent glucoamylase to which it has the highest sequence identity to. In one aspect, the glucoamylase variant exhibits both decreased thermostability and similar or increased specific activity as compared to the parent glucoamylase, such as the parent glucoamylase to which it has the highest sequence identity to.

In one aspect, the glucoamylase variant exhibits altered saccharification performance in the FV measured by the real degree of fermentation (RDF) as compared to the parent glucoamylase. In one aspect, the glucoamylase variant described herein produces similar or decreased RDF value in brewing as compared to the parent glucoamylase, such as the parent glucoamylase to which it has the highest sequence identity to.

In a further aspect, the glucoamylase variant described herein is inactivated by pasteurisation such as using less than 16.8, 16, 15, 14, 13, 12, 11, 10, 9, 8, 7, 6, 5, or 4 pasteurisation units (PU) in beer. In yet a further aspect, the glucoamylase variant has a glucoamylase activity (GAU) of 0.05-10 GAU/mg, such as 0.1-5 GAU/mg, such as 0.5-4 GAU/mg, such as 0.7-4 GAU/mg, or such as 2-4 GAU/mg.

In one aspect, the glucoamylase variant described herein when in its crystal form has a crystal structure for which the atomic coordinates of the main chain atoms have a root-mean-square deviation from the atomic coordinates of the equivalent main chain atoms of TrGA (as defined in Table 20 in WO2009/067218) of less than 0.13 nm following alignment of equivalent main chain atoms, and which have a linker region, a starch binding domain and a catalytic domain.

In one aspect, the glucoamylase variant described herein comprises an amino acid substitution at the residue corresponding to position F29 of SEQ ID NO:2 or an equivalent position in a parent glucoamylase. In one aspect, the glucoamylase variant described herein comprises the following amino acid substitution F29A/R/N/D/C/E/F/G/H/K/S/T/Q/I/L/M/P/V of SEQ ID NO:2, or an equivalent position in a parent glucoamylase. In one aspect, the glucoamylase variant described herein comprises the following amino acid substitution F29V of SEQ ID NO:2, or an equivalent position in a parent glucoamylase. In one aspect, the glucoamylase variant described herein comprises an amino acid substitution at the residue corresponding to position 143 of SEQ ID NO:2, or an equivalent position in a parent glucoamylase. In one aspect, the glucoamylase variant described herein comprises the following amino acid substitution I43Q of SEQ ID NO:2, or an equivalent position in a parent glucoamylase. In one aspect, the glucoamylase variant described herein comprises an amino acid substitution at the residue corresponding to position Y48 of SEQ ID NO:2, or an equivalent position in a parent glucoamylase. In one aspect, the glucoamylase variant described herein comprises In one aspect, the glucoamylase variant described herein comprises the following amino acid substitution Y48V of SEQ ID NO:2, or an equivalent position in a parent glucoamylase. In one aspect, the glucoamylase variant described herein comprises In one aspect, the glucoamylase variant described herein comprises an amino acid substitution at the residue corresponding to position F116 of SEQ ID NO:2, or an equivalent position in a parent glucoamylase. In one aspect, the glucoamylase variant described herein comprises the following amino acid substitution F116M of SEQ ID NO:2, or an equivalent position in a parent glucoamylase. In one aspect, the glucoamylase variant described herein comprises In one aspect, the glucoamylase variant described herein comprises an amino acid substitution at the residue corresponding to position H502 of SEQ ID NO:2, or an equivalent position in a parent glucoamylase. In one aspect, the glucoamylase variant described herein comprises the following amino acid substitution

H502A/N/D/C/E/F/G/H/K/S/T/Q/I/L/M/P/V/W/Y of SEQ ID NO:2, or an equivalent position in a parent glucoamylase. In one aspect, the glucoamylase variant described herein comprises In one aspect, the glucoamylase variant described herein comprises the following amino acid substitution H502S/E of SEQ ID NO:2, or an equivalent position in a parent glucoamylase. In one aspect, the glucoamylase variant described herein comprises an amino acid substitution at the residue corresponding to position S97 of SEQ ID NO:2, or an equivalent position in a parent glucoamylase. In one aspect, the glucoamylase variant described herein comprises the following amino acid substitution S97M of SEQ ID NO:2, or an equivalent position in a parent glucoamylase. In one aspect, the glucoamylase variant described herein comprises an amino acid substitution at the residue corresponding to position L98 of SEQ ID NO:2, or an equivalent position in a parent glucoamylase. In one aspect, the glucoamylase variant described herein comprises the following amino acid substitution
L98A/R/N/E/G/H/K/S/T/Q/I/L/M/P/V/Y of SEQ ID NO:2, or an equivalent position in a parent glucoamylase. In one aspect, the glucoamylase variant described herein comprises the following amino acid substitution L98E of SEQ ID NO:2, or an equivalent position in a parent glucoamylase. In one aspect, the glucoamylase variant described herein comprises an amino acid substitution at the residue corresponding to position Y147 of SEQ ID NO:2, or an equivalent position in a parent glucoamylase. In one aspect, the glucoamylase variant described herein comprises the following amino acid substitution Y147R of SEQ ID NO:2, or an equivalent position in a parent glucoamylase. In one aspect, the glucoamylase variant described herein comprises an amino acid substitution at the residue corresponding to position F175 of SEQ ID NO:2, or an equivalent position in a parent glucoamylase. In one aspect, the glucoamylase variant described herein comprises the following amino acid substitution F175V/I/L of SEQ ID NO:2, or an equivalent position in a parent glucoamylase. In one aspect, the glucoamylase variant described herein comprises an amino acid substitution at the residue corresponding to position G483 of SEQ ID NO:2, or an equivalent position in a parent glucoamylase. In one aspect, the glucoamylase variant described herein comprises the following amino acid substitution G483S of SEQ ID NO:2, or an equivalent position in a parent glucoamylase. In one aspect, the glucoamylase variant described herein comprises an amino acid substitution at the residue corresponding to position T484 of SEQ ID NO:2, or an equivalent position in a parent glucoamylase. In one aspect, the glucoamylase variant described herein comprises the following amino acid substitution T484W of SEQ ID NO:2, or an equivalent position in a parent glucoamylase.

In one aspect, the total number of amino acid substitutions (1) in the group of interface amino acid residues consisting of residues 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596 of SEQ ID NO:2, or an equivalent position in a parent glucoamylase; and (2) in the group of catalytic core amino acid residues consisting of residues not in direct contact with the starch binding domain in positions 1 to 484 with exception of position 24, 26, 27, 29, 30, 40, 42, 43, 44, 46, 48, 49, 110, 111, 112, 114, 116, 117, 118 and 119 of SEQ ID NO: 2, or an equivalent position in a parent glucoamylase; are two, three or four.

In one aspect, the glucoamylase variant described herein comprises the following amino acid substitutions F29V-G483S, Y48V-L98E-H502S, F116M-F175V, F175V-H502E, I43Q-F175I, I43Q-F175V-H502S, F29V-597M-G483S-T484W, or L98E-Y147R-H502S of SEQ ID NO: 2 or 13, or an equivalent position in a parent glucoamylase. In one aspect, the glucoamylase variant described herein further comprises the following amino acid substitutions L417V, T430A, Q511H, A539R and N563I. In one aspect, the glucoamylase variant described herein is SEQ ID NO: 14, 15 or 16. In one aspect, the glucoamylase variant described herein is SEQ ID NO: 14, 15 or 17. In a further aspect, the glucoamylase variant described herein comprises SEQ ID NO: 14, 15 or 16. In a further aspect, the glucoamylase variant described herein comprises SEQ ID NO: 14, 15 or 17.

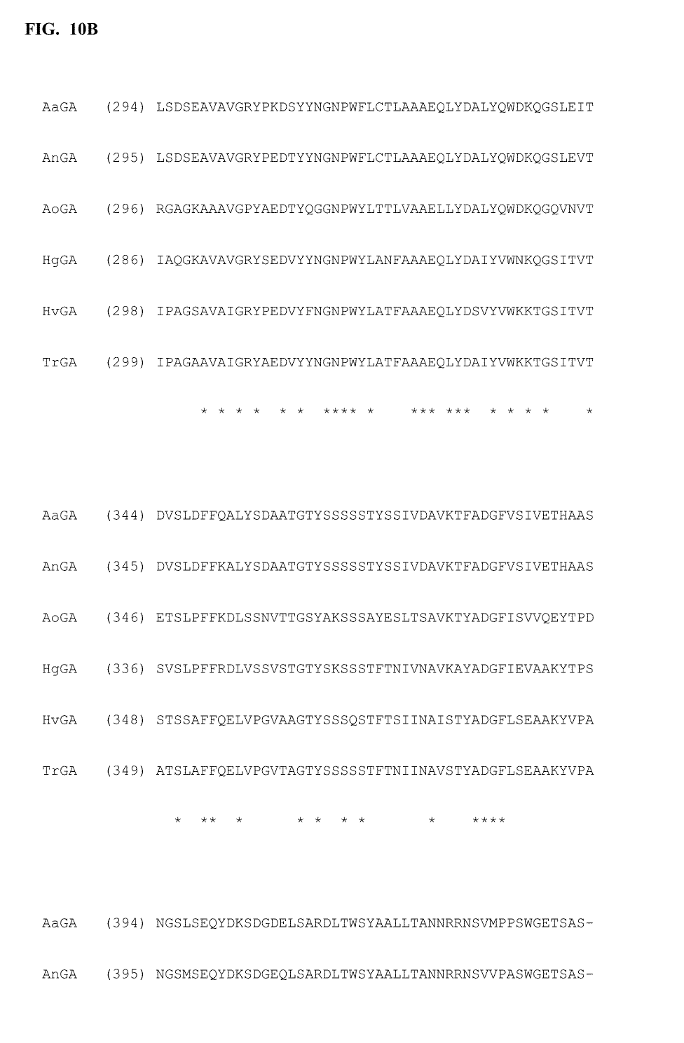

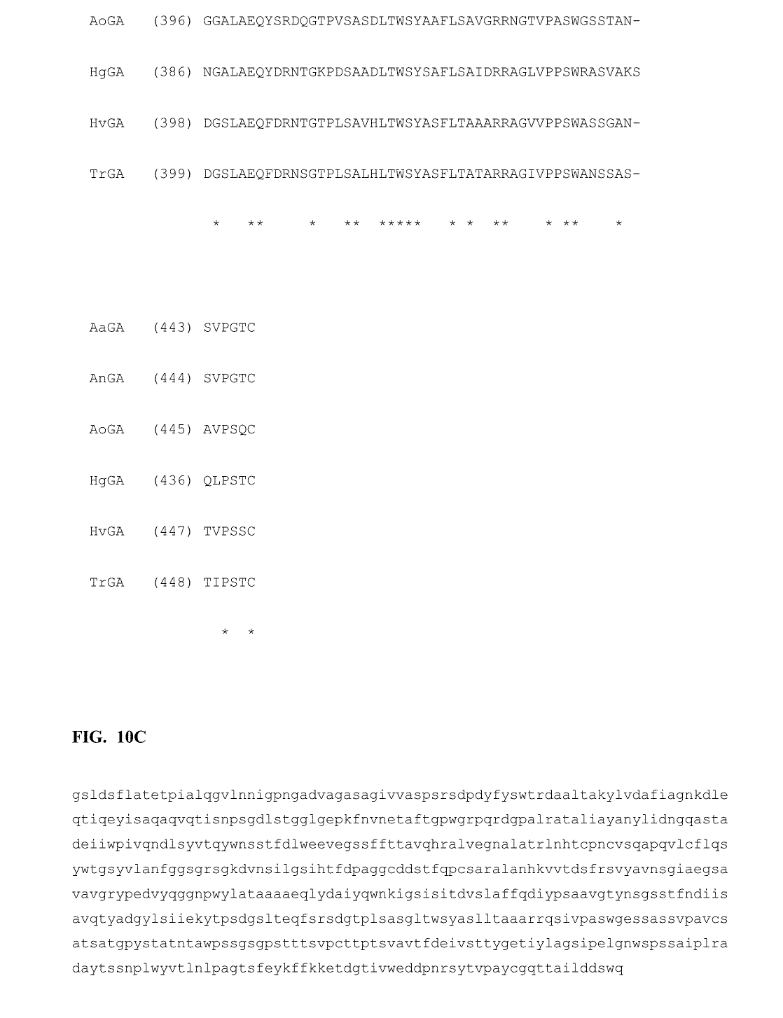

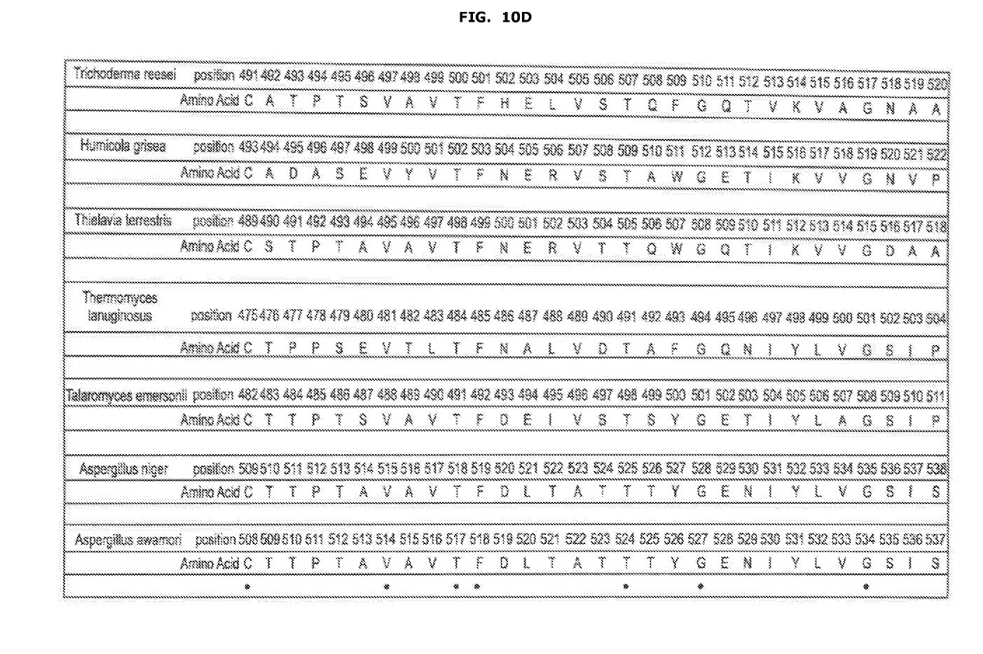

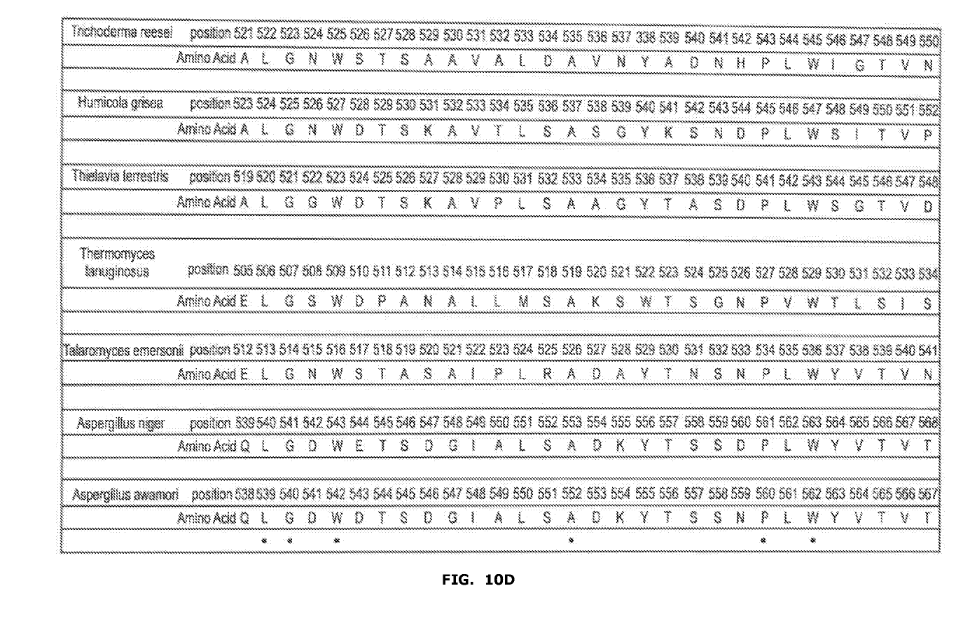

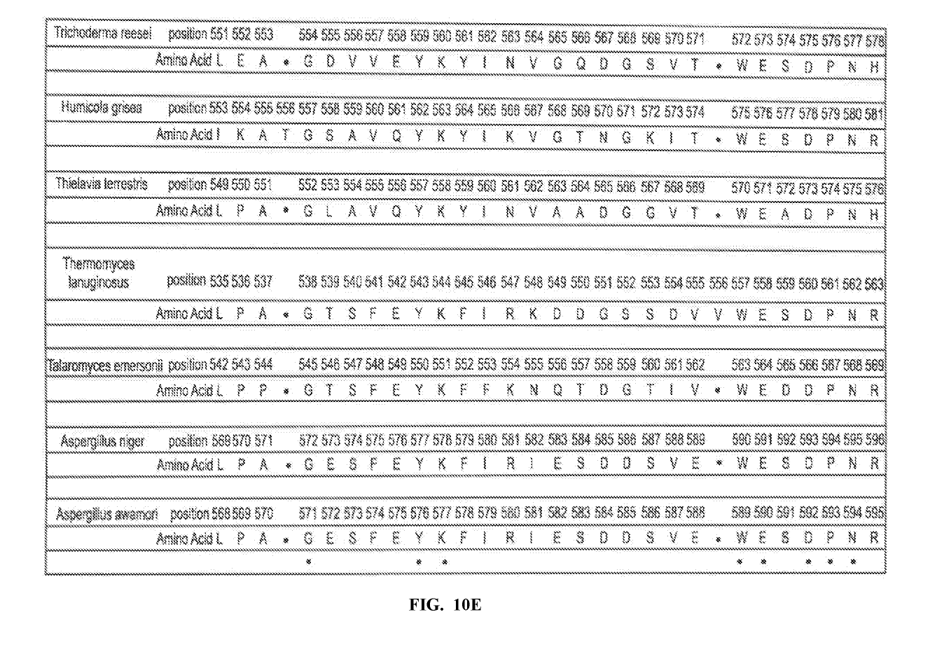

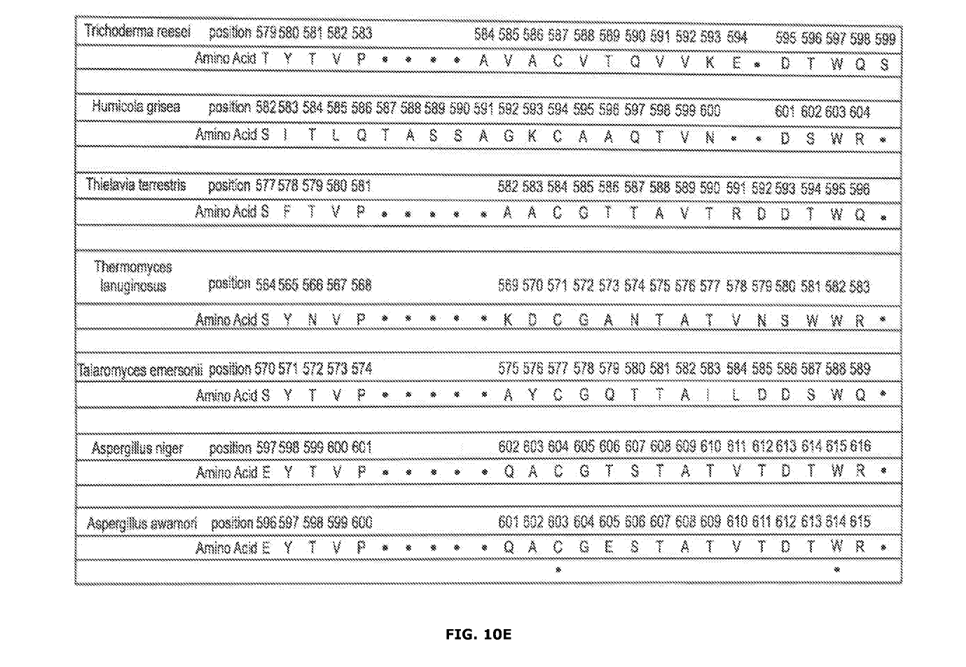

In one aspect, the parent glucoamylase is selected from a glucoamylase obtained from a Trichoderma spp., an Aspergillus spp., a Humicola spp., a Penicillium spp., a Talaromycese spp., or a Schizosaccharmyces spp. (FIGS. 10C, D and E). In a further aspect, the parent glucoamylase is obtained from a Trichoderma spp. or an Aspergillus spp.

In one aspect, the percentage of identity of one amino acid sequence with, or to, another amino acid sequence is determined by the use of the protein-protein Blast search (http://blast.ncbi.nlm.nih.gov) with default settings: score matrix: blosum62, non-redundant protein sequences database and the blast algorithm

| | | |
|---|---|---|
| Settings | Expect threshold | 10 |
| | Max matches in a query range | 0 |
| | Gap opening penalty | 11 |
| | Gap extension penalty | 1 |
| | Compositional adjustment: | |
| | Conditional compositional score | |
| | matrix adjustment | |
| | Mask and filters | No |

In one aspect, the glucoamylase variant is obtained by recombinant expression in a host cell.

In one aspect, the invention relates to a nucleic acid capable of encoding a glucoamylase variant as described herein. In a further aspect, an expression vector or plasmid comprising such a nucleic acid, or capable of expressing a glucoamylase variant as described herein, is disclosed. In one aspect, the expression vector or plasmid comprises a promoter derived from Trichoderma such as a T. reesei cbhI-derived promoter. In a further aspect, the expression vector or plasmid comprises a terminator derived from Trichoderma such as a T. reesei cbhI-derived terminator. In yet a further aspect, the expression vector or plasmid comprises one or more selective markers such as Aspergillus nidulans amdS and pyrG. In another aspect, the expression vector or plasmid comprises one or more telomere regions allowing for a non-chromosomal plasmid maintenance in a host cell.

In one aspect, the invention relates to a host cell having heterologous expression of a glucoamylase variant as herein described. In a further aspect, the host cell according is a fungal cell. In yet a further aspect, the fungal cell is of the genus Trichoderma. In yet a further aspect, the fungal cell is of the species Trichoderma reesei or of the species Hypocrea jecorina. In another aspect, the host cell comprises, preferably transformed with, a plasmid or an expression vector as described herein.

In one aspect, the invention relates to a method of isolating a glucoamylase variant as defined herein, the method comprising the steps of inducing synthesis of the glucoamylase variant in a host cell as defined herein having heterologous expression of said glucoamylase variant and recovering extracellular protein secreted by said host cell, and optionally purifying the glucoamylase variant. In a further aspect, the invention relates to a method for producing a glucoamylase variant as defined herein, the method comprising the steps of inducing synthesis of the glucoamylase variant in a host cell as defined herein having heterologous expression of said glucoamylase variant, and optionally purifying the glucoamylase variant. In a further aspect, the invention relates to a method of expressing a glucoamylase variant as defined herein, the method comprising obtaining a host cell as defined herein and expressing the glucoamylase variant from said host cell, and optionally purifying the glucoamylase variant. In another aspect, the glucoamylase variant as defined herein is the dominant secreted protein.

In one aspect, the invention relates to a composition comprising one or more glucoamylase variant(s) as described herein. In one aspect, the composition is selected from among a starch hydrolyzing composition, a saccharifying composition, a detergent composition, an alcohol fermentation enzymatic composition, and an animal feed animal feed composition. In a further aspect the composition comprises one or more further enzyme(s). In yet a further aspect, the one or more further enzyme(s) is selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. In a further aspect, such glucoamylase variant(s) and/or one or more further enzyme(s) is inactivated by pasteurisation. In yet a further aspect, the glucoamylase variant and/or the one or more further enzyme(s) is inactivated by pasteurisation such as by using less than 50, 45, 40, 35, 30, 25, 24, 23, 22, 21, 20, 19, 18, 17, 16 or 15 pasteurisation units (PU) in beer.

In one aspect, the invention relates to the use of a glucoamylase variant as disclosed herein or a composition as disclosed herein in a fermentation, wherein said glucoamylase variant or composition is added before or during a fermentation step. In a further aspect, said fermentation step, and optional beer filtration step, is followed by a pasteurisation step. In one aspect, said fermentation is comprised in a process for making a fermented beverage. In one aspect, said fermented beverage is selected from the group consisting of beer such as low alcohol beer or low calorie beer. In one aspect, the herein disclosed glucoamylase variant or composition is added in combination with one or more further enzyme(s), such as alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. In a further aspect, the glucoamylase variant and/or the one or more further enzyme(s) is inactivated in the pasteurisation step. In an aspect, the glucoamylase variant is added in an amount of for example 0.01-50 mg pr. ml fermented wort, such as 0.05-25 mg pr. ml fermented wort, such as 0.1-15 mg pr. ml fermented wort, such as 0.2-10 mg pr. ml fermented wort, such as 1-5 mg pr. ml fermented wort. In one aspect, described herein is the use of a thermolabile glucoamylase variant to enhance the production of fermentable sugars in the fermentation step of a brewing process, wherein the glucoamylase variant is as disclosed herein.

In one aspect the invention relates to a method which comprises adding a glucoamylase variant as disclosed herein or a composition as disclosed herein before or during a fermentation step, such as a fermentation step with yeast. In a further aspect, the method comprises a pasteurisation step after the fermentation step or optional beer filtration step. In a further aspect, said fermentation is comprised in a process for making a fermented beverage. In yet a further aspect, said fermented beverage is selected from the group consisting of beer such as low alcohol beer, low calorie beer. In a further aspect, said glucoamylase variant or said composition is added in combination with one or more further enzyme(s) such as selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. In another aspect, the glucoamylase variant and/or the one or more further enzyme(s) is inactivated in the pasteurisation step. In one aspect, the glucoamylase variant is added in an amount of for example 0.01-50 mg pr. ml fermented wort, such as 0.05-25 mg pr. ml fermented wort, such as 0.1-15 mg pr. ml fermented wort, such as 0.2-10 mg pr. ml fermented wort, such as 1-5 mg pr. ml fermented wort. In yet a further aspect, the method for production of a fermented beverage comprises the following steps:
a) preparing a mash,
b) filtering the mash to obtain a wort, and
c) fermenting the wort to obtain a fermented beverage,
   wherein a glucoamylase variant as disclosed herein or a composition as disclosed herein is added to: the mash of step (a) and/or the wort of step (b) and/or the wort of step (c).

In a further aspect, the fermented beverage is subjected to a pasteurisation step (d). In yet a further aspect, the mash in step (a) is obtained from a grist, such as wherein the grist comprises one or more of malted and/or unmalted grain, or starch-based material from another crop. In further aspect, the method further comprises contacting the mash of step (a) with one or more further enzyme(s), such as wherein the enzyme is selected among a starch debranching enzyme, R-enzyme, limit dextrinase, alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. In a further aspect, the method further comprises contacting the wort of step (b) or (c) with one or more further enzyme(s), wherein the enzyme is selected among a starch debranching enzyme, isoamylase and limit dextrinase, including any combinations thereof.

In a further aspect, the invention relates to a fermented beverage wherein the fermented beverage is produced by a method as described herein. In a further aspect, the fermented beverage is beer such as low alcohol beer or low calorie beer.

In a further aspect, the invention relates to a method for the production of a food, feed, or beverage product, such as an alcoholic or non-alcoholic beverage, such as a cereal- or malt-based beverage like beer or whiskey, such as wine, cider, vinegar, rice wine, soya sauce, or juice, said method comprising the step of treating a starch and/or sugar containing plant material with a glucoamylase variant as disclosed herein, or a composition as disclosed herein.

In a further aspect, the invention relates to a kit comprising a glucoamylase variant as disclosed herein, or a composition as disclosed herein; and instructions for use of said glucoamylase variant or composition.

In a further aspect, the invention relates to the use of a glucoamylase variant as disclosed herein, or a composition as disclosed herein, in the production of a first- or second-generation biofuel, such as bioethanol and/or biobutanol.

In a further aspect, the invention relates to the use of a glucoamylase variant as disclosed herein, or a composition as disclosed herein, in the production of a biochemical, such as bio-based isoprene.

In a further aspect, the invention relates to a method for the production of a first- or second-generation biofuel, such as bioethanol and/or biobutanol, said method comprising the step of treating a starch comprising material with a glucoamylase variant as disclosed herein, or a composition as disclosed herein.

In a further aspect, the invention relates to a method for the production of a biochemical, such as bio-based isoprene, said method comprising the step of treating a starch comprising material with a glucoamylase variant as disclosed herein, or a composition as disclosed herein.

In a further aspect, the invention relates to a product obtained by a method according to the invention.

In a further aspect, the invention relates to a composition comprising the product obtained by a method according to the invention, such as wherein the product is in a range of 0.1%-99.9%.

### 1. DEFINITIONS

Unless defined otherwise, all technical and scientific terms used herein have the same meaning as commonly understood by one of ordinary skill in the art to which this disclosure belongs. Singleton et al., Dictionary of Microbiology And Molecular Biology, 2^{nd} ed., John Wiley and Sons, New York (1994), and Hale & Markham, The Harper Collins Dictionary Of Biology, Harper Perennial, N.Y. (1991) provide one of skill with the general meaning of many of the terms used herein. Still, certain terms are defined below for the sake of clarity and ease of reference.

As used herein, the term “glucoamylase” (EC 3.2.1.3) refers to an enzyme that catalyzes the release of D-glucose from the non-reducing ends of starch and related oligo- and polysaccharides.

The term “parent” or “parent sequence” refers to a sequence that is native or naturally occurring in a host cell. Parent glucoamylases include, but are not limited to, the glucoamylase sequences set forth in any one of SEQ ID NOs: 1, 2, 13, 18, 19, 20, 21 and 22, and glucoamylases with at least 80% amino acid sequence identity to SEQ ID NO: 2.

As used herein, the term “parent” or “parent sequence” may also refer to the mature TrGA variant CS4 (SEQ ID NO: 13), including L417V-T430A-Q511H-A539R-N563I compared to TrGA (SEQ ID NO. 2). The mature form of TrGA CS4 includes the catalytic domain, linker region and starch binding domain having the amino acid sequence of SEQ ID NO: 13. The numbering of the glucoamylase amino acids in TrGA CS4 is similar to TrGA and based on the sequence alignment of a glucoamylase with TrGA (SEQ ID NO: 2 and/or 3). The three dimensional structure of TrGA CS4 is expected to be identical to the three dimensional structure of Trichoderma reesei glucoamylase (see Table 20 in WO2009/067218 (Danisco US Inc., Genencor Division) page 94-216 incorporated herein by reference and Example 11 in WO2009/067218 (Danisco US Inc., Genencor Division) page 89-93 incorporated herein by reference).

As used herein, an “equivalent position” means a position that is common to two parent sequences that is based on an alignment of the amino acid sequence of the parent glucoamylase in question as well as alignment of the three-dimensional structure of the parent glucoamylase in question with the TrGA reference glucoamylase amino acid sequence (SEQ ID NO: 2 or 13) and three-dimensional structure. Thus either sequence alignment or structural alignment may be used to determine equivalence.

The term “TrGA” refers to a parent Trichoderma reesei glucoamylase sequence having the mature protein sequence illustrated in SEQ ID NO: 2 that includes the catalytic domain having the sequence illustrated in SEQ ID NO: 3. The isolation, cloning and expression of the TrGA are described in WO 2006/060062 and U.S. Pat. No. 7,413,887, both of which are incorporated herein by reference. In some embodiments, the parent sequence refers to a glucoamylase sequence that is the starting point for protein engineering. The numbering of the glucoamylase amino acids herein is based on the sequence alignment of a glucoamylase with TrGA (SEQ ID NO: 2 and/or 3).

The term “TrGA CS4” or “CS4” refers to the parent Trichoderma reesei glucoamylase variant CS4 sequence having the mature protein sequence illustrated in SEQ ID NO: 13 that includes L417V-T430A-Q511H-A539R-N563I compared to TrGA (SEQ ID NO: 2).

The phrase “mature form of a variant, protein or polypeptide” refers to the final functional form of the variant, protein or polypeptide. A mature form of a glucoamylase may lack a signal peptide, for example. To exemplify, a mature form of the TrGA/-CS4 includes the catalytic domain, linker region and starch binding domain having the amino acid sequence of SEQ ID NO: 2/13.

As used herein, the terms “glucoamylase variant” and “variant” are used in reference to glucoamylases that have some degree of amino acid sequence identity to a parent glucoamylase sequence. A variant is similar to a parent sequence, but has at least one substitution, deletion or insertion in their amino acid sequence that makes them different in sequence from a parent glucoamylase. In some cases, variants have been manipulated and/or engineered to include at least one substitution, deletion, or insertion in their amino acid sequence that makes them different in sequence from a parent. Additionally, a glucoamylase variant may retain the functional characteristics of the parent glucoamylase, e.g., maintaining a glucoamylase activity that is at least about 50%, about 60%, about 70%, about 80%, or about 90% of that of the parent glucoamylase. Can also have higher activity than 100% if that is what one has selected for.

“Variants” may have at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to a parent polypeptide sequence when optimally aligned for comparison. In some embodiments, the glucoamylase variant may have at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to the catalytic domain of a parent glucoamylase. In some embodiments, the glucoamylase variant may have at least at least about 45%, about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, or about 99.5% sequence identity to the starch binding domain of a parent glucoamylase. The sequence identity can be measured over the entire length of the parent or the variant sequence.

As used herein, a “homologous sequence” and “sequence identity” with regard to a nucleic acid or polypeptide sequence means having about at least 100%, at least 99%, at least 98%, at least 97%, at least 96%, at least 95%, at least 94%, at least 93%, at least 92%, at least 91%, at least 90%, at least 88%, at least 85%, at least 80%, at least 75%, at least 70%, at least 65%, at least 60%, at least 55%, at least 50%, or at least 45% sequence identity to a nucleic acid sequence or polypeptide sequence when optimally aligned for comparison, wherein the function of the candidate nucleic acid sequence or polypeptide sequence is essentially the same as the nucleic acid sequence or polypeptide sequence the candidate homologous sequence is being compared with. In some embodiments, homologous sequences have between at least about 85% and 100% sequence identity, while in other embodiments there is between about 90% and 100% sequence identity, and in other embodiments, there is at least about 95% and 100% sequence identity.

### Degree of Identity

The relatedness between two amino acid sequences or between two nucleotide sequences is described by the parameter “identity”.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the reference sequence.

In one embodiment, the degree of sequence identity between a query sequence and a reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the longest of the two sequences.

In another embodiment, the degree of sequence identity between the query sequence and the reference sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the “alignment length”, where the alignment length is the length of the entire alignment including gaps and overhanging parts of the sequences.

Sequence identity comparisons can be conducted by eye, or more usually, with the aid of readily available sequence comparison programs. These commercially available computer programs use complex comparison algorithms to align two or more sequences that best reflect the evolutionary events that might have led to the difference(s) between the two or more sequences. Therefore, these algorithms operate with a scoring system rewarding alignment of identical or similar amino acids and penalising the insertion of gaps, gap extensions and alignment of non-similar amino acids. The scoring system of the comparison algorithms include:
i) assignment of a penalty score each time a gap is inserted (gap penalty score),
ii) assignment of a penalty score each time an existing gap is extended with an extra position (extension penalty score),
iii) assignment of high scores upon alignment of identical amino acids, and
iv) assignment of variable scores upon alignment of non-identical amino acids.
   Most alignment programs allow the gap penalties to be modified. However, it is preferred to use the default values when using such software for sequence comparisons.

The scores given for alignment of non-identical amino acids are assigned according to a scoring matrix also called a substitution matrix. The scores provided in such substitution matrices are reflecting the fact that the likelihood of one amino acid being substituted with another during evolution varies and depends on the physical/chemical nature of the amino acid to be substituted. For example, the likelihood of a polar amino acid being substituted with another polar amino acid is higher compared to being substituted with a hydrophobic amino acid. Therefore, the scoring matrix will assign the highest score for identical amino acids, lower score for non-identical but similar amino acids and even lower score for non-identical non-similar amino acids. The most frequently used scoring matrices are the PAM matrices (Dayhoff et al. (1978), Jones et al. (1992)), the BLOSUM matrices (Henikoff and Henikoff (1992)) and the Gonnet matrix (Gonnet et al. (1992)).

Suitable computer programs for carrying out such an alignment include, but are not limited to, Vector NTI (Invitrogen Corp.) and the ClustalV, ClustalW and ClustalW2 programs (Higgins D G & Sharp P M (1988), Higgins et al. (1992), Thompson et al. (1994), Larkin et al. (2007). A selection of different alignment tools is available from the ExPASy Proteomics server at www.expasy.org. Another example of software that can perform sequence alignment is BLAST (Basic Local Alignment Search Tool), which is available from the webpage of National Center for Biotechnology Information which can currently be found at http://www.ncbi.nlm.nih.gov/ and which was firstly described in Altschul et al. (1990) J. Mol. Biol. 215; 403-410.

In one embodiment of the present invention, the alignment program is performing a global alignment program, which optimizes the alignment over the full-length of the sequences. In a further embodiment, the global alignment program is based on the Needleman-Wunsch algorithm (Needleman, Saul B.; and Wunsch, Christian D. (1970). “A general method applicable to the search for similarities in the amino add sequence of two proteins”. Journal of Molecular Biology 48 (3): 443-53). Examples of current programs performing global alignments using the Needleman-Wunsch algorithm are EMBOSS Needle and EMBOSS Stretcher programs, which are both available at http://www.ebi.ac.uk/Tools/psa/.

EMBOSS Needle performs an optimal global sequence alignment using the Needleman-Wunsch alignment algorithm to find the optimum alignment (including gaps) of two sequences along their entire length.

EMBOSS Stretcher uses a modification of the Needleman-Wunsch algorithm that allows larger sequences to be globally aligned.

In one embodiment, the sequences are aligned by a global alignment program and the sequence identity is calculated by identifying the number of exact matches identified by the program divided by the “alignment length”, where the alignment length is the length of the entire alignment including gaps and overhanging parts of the sequences. In a further embodiment, the global alignment program uses the Needleman-Wunsch algorithm and the sequence identity is calculated by identifying the number of exact matches identified by the program divided by the “alignment length”, where the alignment length is the length of the entire alignment including gaps and overhanging parts of the sequences.

In yet a further embodiment, the global alignment program is selected from the group consisting of EMBOSS Needle and EMBOSS stretcher and the sequence identity is calculated by identifying the number of exact matches identified by the program divided by the “alignment length”, where the alignment length is the length of the entire alignment including gaps and overhanging parts of the sequences.

Once the software has produced an alignment, it is possible to calculate % similarity and % sequence identity. The software typically does this as part of the sequence comparison and generates a numerical result.

In one embodiment, it is preferred to use the ClustalW software for performing sequence alignments. Preferably, alignment with ClustalW is performed with the following parameters for pairwise alignment:

| | |
|---|---|
| Substitution matrix: | Gonnet 250 |
| Gap open penalty: | 20 |
| Gap extension penalty: | 0.2 |
| Gap end penalty: | None |

ClustalW2 is for example made available on the internet by the European Bioinformatics Institute at the EMBL-EBI webpage www.ebi.ac.uk under tools—sequence analysis—ClustalW2. Currently, the exact address of the ClustalW2 tool is www.ebi.ac.uK/Tools/clustalw2.

In another embodiment, it is preferred to use the program Align X in Vector NTI (Invitrogen) for performing sequence alignments. In one embodiment, Exp10 has been may be used with default settings:

Gap opening penalty: 10
Gap extension penalty: 0.05
Gap separation penalty range: 8

In a another embodiment, the alignment of one amino acid sequence with, or to, another amino acid sequence is determined by the use of the score matrix: blosum62mt2 and the VectorNT1 Pair wise alignment settings

| | | |
|---|---|---|
| Settings | K-tuple | 1 |
| | Number of best diagonals | 5 |
| | Window size | 5 |
| | Gap Penalty | 3 |
| | Gap opening Penalty | 10 |
| | Gap extension Penalty | 0.1 |

In a preferred embodiment, the percentage of identity of one amino acid sequence with, or to, another amino acid sequence is determined by the use of the protein-protein Blast search (http://blast.ncbi.nlm.nih.gov) with default settings: score matrix: word size of 3, blosum62 substitution matrix, non-redundant protein sequences database and the blast algorithm

| | | |
|---|---|---|
| Settings | Expect threshold | 10 |
| | Max matches in a query range | 0 |
| | Gap opening penalty | 11 |
| | Gap extension penalty | 1 |
| | Compositional adjustment: | |
| | Conditional compositional score | |
| | matrix adjustment | |
| | Mask and filters | No |

The term “optimal alignment” refers to the alignment giving the highest percent identity score.

Homology is determined using standard techniques known in the art (see e.g., Smith and Waterman, Adv. Appl. Math. 2: 482 (1981); Needleman and Wunsch, J. Mol. Biol. 48: 443 (1970); Pearson and Lipman, Proc. Natl. Acad. Sci. USA 85: 2444 (1988); programs such as GAP, BESTHT, FASTA, and TFASTA in the Wisconsin Genetics Software Package (Genetics Computer Group, Madison, Wis.); and Devereux el al., Nucleic Acid Res., 12: 387-395 (1984)).

Homologous sequences are determined by known methods of sequence alignment. “Sequence identity” is determined herein by the method of sequence alignment. A commonly used alignment method is BLAST described by Altschul et al., (Altschul et al., J. Mol. Biol. 215: 403-410 (1990); and Karlin et al, Proc. Natl. Acad. Sci. USA 90: 5873-5787 (1993)). A particularly useful BLAST program is the WU-BLAST-2 program (see Altschul et al, Meth. Enzymol. 266: 460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched. However, the values may be adjusted to increase sensitivity.

Other methods find use in aligning sequences. One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (Feng and Doolittle, J. Mol. Evol. 35: 351-360 (1987)). The method is similar to that described by Higgins and Sharp (Higgins and Sharp, CABIOS 5: 151-153 (1989)). Useful PILEUP parameters including a default gap weight of 3.00, a default gap length weight of 0.10, and weighted end gaps.

As used herein, the terms “glucoamylase variant” or “variant” are used in reference to glucoamylases that are similar to a parent glucoamylase sequence but have at least one substitution, deletion, or insertion in their amino acid sequence that makes them different in sequence from the parent sequence. In some cases, they have been manipulated and/or engineered to include at least one substitution, deletion, or insertion in their amino acid sequence that makes them different in sequence from the parent glucoamylase.

As used herein the term “catalytic domain” refers to a structural region of a polypeptide, which contains the active site for the catalysis of substrate hydrolysis, see for example the specified region of TrGA below.

The interface region between the catalytic core domain and the starch binding domain in the glucoamylase from Trichoderma reesei was determined by the use of the PDBePISA interactive tool for the exploration of macromolecular protein interfaces (http://www.ebi.ac.uk/msd-srv/prot_int/pistart.html) using database search and PDB entry parameter: 2VN4 (R. Bott et al., (2008) Biochemistry 47: 5746-5754) chain identity modified for intra-molecular interface analysis by: chain A, residue 1-453; catalytic core domain and chain B, residue 491-599. Interface search was performed with default settings for interface analysis:

| | | |
|---|---|---|
| Settings: | Processing mode, | Auto |
| | Processing of ligands, | yes |

The search resulted in the following amino acid residues at the connecting surface area between the two domains, corresponding to positions 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596 of SEQ ID NO: 2, that were validated by manual inspection using Pymol (The PyMOL Molecular Graphics System, Version 1.2r3pre, Schrödinger, LLC.).

In the present context the term “residues not in direct contact with the starch binding domain in positions 1 to 484” means amino acid residues in positions 1 to 484 of SEQ ID NO: 2 which has no direct electrostatic, polar or hydrophobic interaction with amino acid residues in the starch binding domain. The majority a residues in positions 1 to 484 are not in direct contact, as seen from the structure of TrGA (PDB ID: 2VN4). The identity of interaction and residues involved may be defined by the PISA ePDB server and consist of: hydrophobic interaction (Van der Waals), hydrogen bonds, dipol or other direct electrostatic interactions between side chain or main chain atoms. Thus, in one aspect, all residues from 1 to 484 of SEQ ID NO: 2 excluding residues: 24, 26, 27, 29, 30, 40, 42, 43, 44, 46, 48, 49, 110, 111, 112, 114, 116, 117, 118 and 119 of SEQ ID NO: 2 are not in direct contact with the starch binding domain.

The term “linker” refers to a short amino acid sequence generally having between 3 and 40 amino acids residues that covalently bind an amino acid sequence comprising a starch binding domain with an amino acid sequence comprising a catalytic domain.

The term “starch binding domain” (SBD) refers to an amino acid sequence that binds preferentially to a starch substrate. It is well known for a person skilled in the art how to identify a SBD—the SBD is an example of a carbohydrate-binding modules (CBM) and CBMs have been classified into the CBM families using a sequence-based classification system (http://www.cazy.org/Carbohydrate-Binding-Modules.html) In addition, it is well known for a person skilled in the art to isolate materials containing for example an SBD using raw starch or beta-cyclodextrin affinity chromatography (Hamilton et al. (2000) Enzyme and Microbial Technology 26 p 561-567). In one aspect, the domain definition of SBD is adopted from the Pfam database (http://pfam.sanger.ac.uk/ or www.sanger.ac.uk/resources/databases/pfam.html) which database of protein domain families are generated from sequence similarity. Thus, in one aspect the SBD is as defined by the Carbohydrate binding module 20 family in the Pfam database.

As used herein, the term “fragment” is defined as a variant having one or more (several) amino acids deleted from the amino and/or carboxyl terminus for example of the polypeptide of SEQ ID NO:2; wherein the fragment has glucoamylase activity. In one aspect, the fragment has one or more (several) amino acids deleted from the amino and/or carboxy terminus of SEQ ID NO:2 or 13.

As used herein the term “truncated” refers to a polypeptide that compared to the parent glucoamylase (or another variant) does not achieve its full translated length and is therefore missing some of the amino acids present in the parent glucoamylase. Truncation is normally brought about by a premature termination mutation, but could be caused by another mechanism—such as a post-translational modification or protease cleavage.

As used herein, the terms “mutant sequence” and “mutant gene” are used interchangeably and refer to a polynucleotide sequence that has an alteration in at least one codon occurring in a host cell's parent sequence. The expression product of the mutant sequence is a variant protein with an altered amino acid sequence relative to the parent glucoamylase. The expression product may have an altered functional capacity (e.g., enhanced enzymatic activity or reduced thermostability).

The term “property” or grammatical equivalents thereof in the context of a polypeptide, as used herein, refers to any characteristic or attribute of a polypeptide that can be selected or detected. These properties include, but are not limited to, oxidative stability, substrate specificity, catalytic activity, thermal stability, pH activity profile, resistance to proteolytic degradation, K_{M}, K_{CAT}, K_{CAT}/K_{M} ratio, protein folding, ability to bind a substrate and ability to be secreted.

The term “property” of grammatical equivalent thereof in the context of a nucleic acid, as used herein, refers to any characteristic or attribute of a nucleic acid that can be selected or detected. These properties include, but are not limited to, a property affecting gene transcription (e.g., promoter strength or promoter recognition), a property affecting RNA processing (e.g., RNA splicing and RNA stability), a property affecting translation (e.g., regulation, binding of mRNA to ribosomal proteins).

The terms “thermally stable” and “thermostable” refer to glucoamylase variants of the present disclosure that retain a specified amount of enzymatic activity after exposure to a temperature over a given period of time under conditions prevailing during the hydrolysis of starch substrates, for example, while exposed to altered temperatures.

The term “enhanced stability” in the context of a property such as thermostability refers to a higher retained catalytic activity, or starch hydrolytic activity however measured, over time as compared to the parent glucoamylase.

The term “thermolabile glucoamylase” refers to a glucoamylase of the present disclosure that loses detectable hydrolytic enzymatic activity after exposure to a temperature over a given period of time. In one aspect, the term “thermolabile glucoamylase” refers to a glucoamylase of the present disclosure that loses detectable hydrolytic enzymatic activity after exposure to a temperature over a given period of time under conditions prevailing during pasteurisation of the product of a brewing process. The precise conditions of pasteurization (e.g. Pasteurization Units) will depend on the type of beer produced by the brewing process. Loss of detectable hydrolytic activity of the thermolabile glucoamylase in a pasteurized beer may be detected using a glucoamylase enzyme assay as described herein and defined by loss of activity measured by that assay. In one aspect, “decreased thermostability” is used interchangelably with “more thermolabile” when comparing to a parent glucoamylase.

The term “specific activity” is defined as the activity per mg of glucoamylase protein. In some embodiments, the activity for glucoamylase is determined by a specific chromogenic glucoamylase assay with a pNP-β-maltoside substrate and expressed as the amount of p-nitrophenol that is produced from the substrate per min under defined assay conditions. In some embodiments, the protein concentration can be determined using the Bradford assay.

The terms “active” and “biologically active” refer to a biological activity associated with a particular protein. It follows that the biological activity of a given protein refers to any biological activity typically attributed to that protein by those skilled in the art. For example, an enzymatic activity associated with a glucoamylase is hydrolytic and, thus an active glucoamylase has hydrolytic activity.

As used herein, the term “glucoamylase activity” refers to the activity of an enzyme that catalyzes the release of D-glucose from the non-reducing ends of starch and related oligo- and polysaccharides. In particular, glucoamylase activity may be assayed by the 3,5-dinitrosalicylic acid (DNS) method (see Goto et al., Biosci. Biotechnol. Biochem. 58:49-54 (1994)).

The terms “polynucleotide” and “nucleic acid”, used interchangeably herein, refer to a polymeric form of nucleotides of any length, either ribonucleotides or deoxyribonucleotides. These terms include, but are not limited to, a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases.

As used herein, the terms “DNA construct,” “transforming DNA” and “expression vector” are used interchangeably to refer to DNA used to introduce sequences into a host cell or organism. The DNA may be generated in vitro by PCR or any other suitable technique(s) known to those in the art. The DNA construct, transforming DNA, or recombinant expression cassette can be incorporated into a plasmid, chromosome, mitochondrial DNA, plastid DNA, virus, or nucleic acid fragment. Typically, the recombinant expression cassette portion of an expression vector, DNA construct, or transforming DNA includes, among other sequences, a nucleic acid sequence to be transcribed, and a promoter. In some embodiments, expression vectors have the ability to incorporate and express heterologous DNA fragments in a host cell.

As used herein, the term “vector” refers to a polynucleotide construct designed to introduce nucleic acids into one or more cell types. Vectors include cloning vectors, expression vectors, shuttle vectors, plasmids, cassettes, and the like.

As used herein in the context of introducing a nucleic acid sequence into a cell, the term “introduced” refers to any method suitable for transferring the nucleic acid sequence into the cell. Such methods for introduction include but are not limited to protoplast fusion, transfection, transformation, conjugation, and transduction.

As used herein, the terms “transformed” and “stably transformed” refers to a cell that has a non-native (heterologous) polynucleotide sequence integrated into its genome or as an episomal plasmid that is maintained for at least two generations.

As used herein, the terms “selectable marker” and “selective marker” refer to a nucleic acid (e.g., a gene) capable of expression in host cells that allows for ease of selection of those hosts containing the vector. Typically, selectable markers are genes that confer antimicrobial resistance or a metabolic advantage on the host cell to allow cells containing the exogenous DNA to be distinguished from cells that have not received any exogenous sequence during the transformation.

As used herein, the term “promoter” refers to a nucleic acid sequence that functions to direct transcription of a downstream gene. The promoter, together with other transcriptional and translational regulatory nucleic acid sequences (also termed “control sequences”) is necessary to express a given gene. In general, the transcriptional and translational regulatory sequences include, but are not limited to, promoter sequences, ribosomal binding sites, transcriptional start and stop sequences, translational start and stop sequences, and enhancer or activator sequences.

A nucleic acid is “operably linked” when it is placed into a functional relationship with another nucleic acid sequence. For example, DNA encoding a secretory leader (i.e., a signal peptide), can be operably linked to DNA for a polypeptide if it is expressed as a preprotein that participates in the secretion of the polypeptide. Generally, “operably linked” means that the DNA sequences being linked are contiguous, and, in the case of a secretory leader, contiguous and in reading phase.

As used herein the term “gene” refers to a polynucleotide (e.g., a DNA segment), that encodes a polypeptide and includes regions preceding and following the coding regions, as well as intervening sequences (introns) between individual coding segments (exons).

As used herein, the term “hybridization” refers to the process by which a strand of nucleic acid joins with a complementary strand through base pairing, as known in the art.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm −5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while an intermediate or low stringency hybridization can be used to identify or detect polynucleotide sequence homologs.

Moderate and high stringency hybridization conditions are well known in the art. An example of high stringency conditions includes hybridization at about 42° C. in 50% formamide, 5 x SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. An example of moderate stringent conditions include an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denaturated sheared salmon sperm DNA, followed by washing the filters in 1×SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous or homologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

In one embodiment, mutated DNA sequences are generated with site saturation mutagenesis in at least one codon and/or nucleotide. In another embodiment, site saturation mutagenesis is performed for two or more codons. In a further embodiment, mutant DNA sequences have more than about 50%, more than 55%, more than 60%, more than 65%, more than 70%, more than 75%, more than 80%, more than 85%, more than 90%, more than 95%, or more than 98% identity with the glucoamylase DNA sequence. In alternative embodiments, mutant DNA can be generated in vivo using any known mutagenic procedure such as, for example, radiation, nitrosoguanidine, and the like. The desired DNA sequence can then be isolated and used in the methods provided herein.

As used herein, “heterologous protein” refers to a protein or polypeptide that does not naturally occur in the host cell.

An enzyme is “over-expressed” in a host cell if the enzyme is expressed in the cell at a higher level than the level at which it is expressed in a corresponding wild-type cell.

The terms “protein” and “polypeptide” are used interchangeability herein. In the present disclosure and claims, the conventional one-letter and three-letter codes for amino acid residues are used. The 3-letter code for amino acids as defined in conformity with the IUPAC-IUB Joint Commission on Biochemical Nomenclature (JCBN). It is also understood that a polypeptide may be coded for by more than one nucleotide sequence due to the degeneracy of the genetic code.

Variants of the disclosure are described by the following nomenclature: [original amino acid residue/position/substituted amino acid residue]. When a position suitable for substitution is identified herein without a specific amino acid suggested, it is to be understood that any amino acid residue may be substituted for the amino acid residue present in the position.

A “prosequence” is an amino acid sequence between the signal sequence and mature protein that is necessary for the secretion of the protein. Cleavage of the pro sequence will result in a mature active protein.

The term “signal sequence” or “signal peptide” refers to any sequence of nucleotides and/or amino acids that may participate in the secretion of the mature or precursor forms of the protein. This definition of signal sequence is a functional one, meant to include all those amino acid sequences encoded by the N-terminal portion of the protein gene, which participate in the effectuation of the secretion of protein. They are often, but not universally, bound to the N-terminal portion of a protein or to the N-terminal portion of a precursor protein. The signal sequence may be endogenous or exogenous. The signal sequence may be that normally associated with the protein (e.g., glucoamylase), or may be from a gene encoding another secreted protein.

The term “precursor” form of a protein or peptide refers to a mature form of the protein having a prosequence operably linked to the amino or carbonyl terminus of the protein. The precursor may also have a “signal” sequence operably linked, to the amino terminus of the prosequence. The precursor may also have additional polynucleotides that are involved in post-translational activity (e.g., polynucleotides cleaved therefrom to leave the mature form of a protein or peptide).

“Host strain” or “host cell” refers to a suitable host for an expression vector comprising DNA according to the present disclosure.

The terms “derived from” and “obtained from” refer to not only a glucoamylase produced or producible by a strain of the organism in question, but also a glucoamylase encoded by a DNA sequence isolated from such strain and produced in a host organism containing such DNA sequence. Additionally, the term refers to a glucoamylase that is encoded by a DNA sequence of synthetic and/or cDNA origin and that has the identifying characteristics of the glucoamylase in question.

A “derivative” within the scope of this definition generally retains the characteristic hydrolyzing activity observed in the glucoamylase to the extent that the derivative is useful for similar purposes as the wild-type, native or parent form. Functional derivatives of glucoamylases encompass naturally occurring, synthetically or recombinantly produced peptides or peptide fragments that have the general characteristics of the glucoamylases of the present disclosure.

The term “isolated” refers to a material that is removed from the natural environment if it is naturally occurring. A “purified” protein refers to a protein that is at least partially purified to homogeneity. In some embodiments, a purified protein can be more than about 10% pure, optionally more than about 20% pure, and optionally more than about 30% pure, as determined by SDS-PAGE. Further aspects of the disclosure encompass the protein in a highly purified form (i.e., more than about 40% pure, more than about 60% pure, more than about 80% pure, more than about 90% pure, more than about 95% pure, more than about 97% pure, and even more than about 99% pure), as determined by SDS-PAGE.

As used herein, the term, “combinatorial mutagenesis” refers to methods in which libraries of variants of a starting sequence are generated. In these libraries, the variants contain one or several mutations chosen from a predefined set of mutations. In addition, the methods provide means to introduce random mutations that were not members of the predefined set of mutations. In some embodiments, the methods include those set forth in U.S. Pat. No. 6,582,914, hereby incorporated by reference. In alternative embodiments, combinatorial mutagenesis methods encompass commercially available kits (e.g., QuikChange® Multisite, Stratagene, San Diego, Calif.).

As used herein, the term “library of mutants” refers to a population of cells that are identical in most of their genome but include different homologues of one or more genes. Such libraries can be used, for example, to identify genes or operons with improved traits.

As used herein the term “dry solids content (DS or ds)” refers to the total solids of a slurry in % on a dry weight basis.

As used herein, the term “initial hit” refers to a variant that was identified by screening a combinatorial consensus mutagenesis library. In some embodiments, initial hits have improved performance characteristics, as compared to the starting gene.

As used herein, the term “improved hit” refers to a variant that was identified by screening an enhanced combinatorial consensus mutagenesis library.

As used herein, the term “target property” refers to the property of the starting gene that is to be altered. It is not intended that the present disclosure be limited to any particular target property. However, in some embodiments, the target property is the stability of a gene product (e.g., resistance to denaturation, proteolysis or other degradative factors), while in other embodiments, the level of production in a production host is altered. Indeed, it is contemplated that any property of a starting gene will find use in the present disclosure. Other definitions of terms may appear throughout the specification.

As used herein the term “composition” relates to a preparation in the form of for example a beverage, food or feed ingredient prepared according to the present invention, and may be in the form of a solution or as a solid—depending on the use and/or the mode of application and/or the mode of administration. The solid form can be either as a dried enzyme powder or as a granulated enzyme. The composition may comprise a variant according to the invention, an enzyme carrier and optionally a stabilizer and/or a preservative. The enzyme carrier may be selected from the group consisting of glycerol or water. The preparation may comprise a stabilizer. The stabilizer may be selected from the group consisting of inorganic salts, polyols, sugars and combinations thereof. Further, the stabilizer may be an inorganic salt such as potassium chloride. In another aspect, the polyol is glycerol, propylene glycol, or sorbitol. The sugar is a small-molecule carbohydrate, in particular any of several sweet-tasting ones such as glucose, fructose and saccharose. In yet at further aspect, the preparation may comprise a preservative. In one aspect, the preservative is methyl paraben, propyl paraben, benzoate, sorbate or other food approved preservatives or a mixture thereof.

In the present context, the term “fermentation” refers to providing a composition such as a fermented beverage and/or substance by growing microorganisms in a culture. In the context of enzyme (e.g. glucoamylase) production, the term “fermentation” refers to a process involving the production of the enzyme in a microbial culture process. In the context of brewing, the term “fermentation” refers to transformation of sugars in a wort, by enzymes in the brewing yeast, into ethanol and carbon dioxide with the formation of other fermentation by-products.

As used herein, the “process for production of a fermented beverage” such as beer comprises in general a step of preparing a mash such as based on a grist, filtering the mash to obtain a wort and spent grain, and fermenting the wort to obtain a fermented beverage.

As used herein the term “starch and/or sugar containing plant material” refers to starch and/or sugar containing plant material derivable from any plant and plant part, including tubers, roots, stems, leaves and seeds. “Starch and/or sugar comprising plant material” can e.g. be one or more cereal, such as barley, wheat, maize, rye, sorghum, millet, or rice, and any combination thereof. The starch- and/or sugar comprising plant material can be processed, e.g. milled, malted, partially malted or unmalted. Unmalted cereal is also called “raw grain”. Examples of non-cereal starch-containing plant material comprise e.g. tubers,

As used herein, the term “grist” refers to any processed starch and/or sugar containing plant material suitable for mashing. The grist, as contemplated herein, may comprise any starch and/or sugar containing plant material derivable from any plant and plant part, including tubers, roots, stems, leaves and seeds. Examples of processing comprise milling and/or grinding, usually providing a material that is more coarse than flour. In the present context grist may comprise processed material from grain, such as grain from barley, wheat, rye, oat, corn (maize), rice, milo, millet and sorghum, and more preferably, at least 10%, or more preferably at least 15%, even more preferably at least 25%, or most preferably at least 35%, such as at least 50%, at least 75%, at least 90% or even 100% (w/w) of the grist of the wort is derived from grain. In some embodiments the grist may comprise the starch and/or sugar containing plant material obtained from cassava [Manihot esculenta] roots. The grist may comprise malted grain, such as barley malt. Preferably, at least 10%, or more preferably at least 15%, even more preferably at least 25%, or most preferably at least 35%, such as at least 50%, at least 75%, at least 90% or even 100% (w/w) of the grist of the wort is derived from malted grain.

As used herein the term “malt” is understood as any malted cereal grain, such as malted barley or wheat.

In one aspect, when using malt produced principally from selected varieties of barley in connection with production of beer, the malt has the greatest effect on the overall character and quality of the beer. First, the malt is the primary flavoring agent in beer. Second, the malt provides the major portion of the fermentable sugar. Third, the malt provides the proteins, which will contribute to the body and foam character of the beer. Fourth, the malt provides enzymatic activities during mashing, optionally complemented by addition of exogenous enzymes. Fifth, the malt spent grains provide a filtration medium for the separation of the wort after mashing—typically by lautering or mash filtration.

As used herein the term “adjunct” refers to any starch and/or sugar containing plant material which is not barley malt. As examples of adjuncts, mention can be made of materials such as common corn (maize) grits, refined corn (maize) grits, brewer's milled yeast, rice, sorghum, refined corn (maize) starch, barley, barley starch, dehusked barley, wheat, wheat starch, torrified cereal, cereal flakes, rye, oats, potato, tapioca, and syrups, such as corn (maize) syrup, sugar cane syrup, inverted sugar syrup, barley and/or wheat syrups, and the like may be used as a source of starch. The starch will eventually be converted into dextrins and fermentable sugars. In one aspect, “adjunct” includes the starch and/or sugar containing plant material obtained from cassava [Manihot esculenta] roots.

As used herein, the term “mash” refers to an aqueous slurry of any starch and/or sugar containing plant material such as grist, e. g. comprising crushed barley malt, crushed barley, and/or other adjunct or a combination hereof, mixed with water later to be separated into wort and spent grains.

As used herein, the term “wort” refers to the unfermented liquor run-off following extracting the grist during mashing.

As used herein, the term “spent grains” refers to the drained solids remaining when the grist has been extracted and the wort is separated from the mash. “Spent grains” can be used e.g. as feed.

As used herein, the term “extract recovery” in the wort refers to the sum of soluble substances extracted from the grist (malt and/or adjuncts) expressed in percentage based on dry matter.

As used herein, the term “hops” refers to its use in contributing significantly to beer quality, including flavoring. In particular, hops (or hops constituents) add desirable bittering substances to the beer. In addition, the hops may act as protein precipitant, establish preservative agents and aid in foam formation and stabilization.

As used herein, the terms “beverage(s)” and “beverage(s) product” includes beers such as full malted beer, beer brewed under the “Reinheitsgebot”, ale, IPA, lager, bitter, Happoshu (second beer), third beer, dry beer, near beer, light beer, low alcohol beer, low calorie beer, porter, bock beer, stout, malt liquor, non-alcoholic beer, non-alcoholic malt liquor and the like. The term “beverage(s)” or “beverages product” also includes alternative cereal and malt beverages such as fruit flavoured malt beverages, e. g., citrus flavoured, such as lemon-, orange-, lime-, or berry-flavoured malt beverages, liquor flavoured malt beverages, e. g., vodka-, rum-, or tequila-flavoured malt liquor, or coffee flavoured malt beverages, such as caffeine-flavoured malt liquor, and the like. In a further aspect, the beverage or beverage product is an alcoholic or non-alcoholic beverage, such as a cereal- or malt-based beverage like beer or whiskey, such as wine, cider, vinegar, rice wine, soya sauce, or juice.

As used herein, the term “malt beverage” includes such malt beverages as full malted beer, ale, IPA, lager, bitter, Happoshu (second beer), third beer, dry beer, near beer, light beer, low alcohol beer, low calorie beer, porter, bock beer, stout, malt liquor, non-alcoholic malt liquor and the like. The term “malt beverages” also includes alternative malt beverages such as fruit flavored malt beverages, e. g., citrus flavored, such as lemon-, orange-, lime-, or berry-flavored malt beverages, liquor flavored malt beverages, e. g., vodka-, rum-, or tequila-flavored malt liquor, or coffee flavored malt beverages, such as caffeine-flavored malt liquor, and the like.

In the context of the present invention, the term “beer” is meant to comprise any fermented wort, produced by fermentation/brewing of a starch-containing plant material, thus in particular also beer produced exclusively from malt or adjunct, or any combination of malt and adjunct.

Beer can be made from a variety of starch and/or sugar containing plant material, often cereal grains and/or malt by essentially the same process. Grain starches are believed to be glucose homopolymers in which the glucose residues are linked by either alpha-1, 4- or alpha-1,6-bonds, with the former predominating.

As used herein, the term “Pilsner beer” refers to a pale bottom-fermented lager (made from Pilsner malt) usually with a more pronounced hop character than normal (e.g. helles) pale lagers.

As used herein, the term “light beers, reduced calorie beers or low calorie beers”, refers to the recent, widespread popularization of brewed beverages, particularly in the U. S. market. As defined in the U. S., these highly attenuated beers have approximately 30% fewer calories than a manufacturer's “normal” beer.”

As used herein, the term “non-alcoholic beer” or “low-alcohol beer” refers to a beer containing a maximum of 0.1%, 0.2%, 0.3%, 0.4%, 0.5% alcohol by volume. Non-alcoholic beer may be brewed by special methods (stopped fermentation), with special non-alcohol producing “yeasts” or by traditional methods, but during the finishing stages of the brewing process the alcohol is removed e.g. by vacuum evaporation, by taking advantage of the different boiling points of water and alcohol.

As used herein, the term “low-calorie beer” or “beer with a low carbohydrate content (low-carb)” is defined as a beer with a carbohydrate content of 0.75 g/100 g or less and with fermentation degree of around 90-92%.

As used herein, the term “pasteurisation” means the killing of micro-organisms in aqueous solution by heating. Implementation of pasteurisation in the brewing process is typically through the use of a flash pasteuriser or tunnel pasteuriser. As used herein, the term “pasteurisation units or PU” refers to a quantitative measure of pasteurisation. One pasteurisation unit (1 PU) for beer is defined as a heat retention of one minute at 60 degrees Celsius. One calculates that:

PU=t×1.393̂(T−60), where:
t=time, in minutes, at the pasteurisation temperature in the pasteuriser
T=temperature, in degrees Celsius, in the pasteuriser
[̂(T−60) represents the exponent of (T−60)]

Different minimum PU may be used depending on beer type, raw materials and microbial contamination, brewer and perceived effect on beer flavour. Typically, for beer pasteurisation, 14-15 PU are required. Depending on the pasteurising equipment, pasteurisation temperatures are typically in the range of 64-72 degrees Celsius with a pasteurisation time calculated accordingly. Further information may be found in “Technology Brewing and Malting” by Wolfgang Kunze of the Research and Teaching Institute of Brewing, Berlin (VLB), 3rd completely updated edition, 2004, ISBN 3-921690-49-8.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Before the exemplary embodiments are described in more detail, it is to be understood that this disclosure is not limited to particular embodiments described, as such may, of course, vary. Although any methods and materials similar or equivalent to those described herein can be used in the practice or testing of the present disclosure, exemplary methods, and materials are now described.

As used herein and in the appended claims, the singular forms “a”, “an”, and “the” include plural referents unless the context clearly dictates otherwise. Thus, for example, reference to “a gene” includes a plurality of such candidate agents and reference to “the cell” includes reference to one or more cells and equivalents thereof known to those skilled in the art, and so forth.

The publications discussed herein are provided solely for their disclosure prior to the filing date of the present application. Nothing herein is to be construed as an admission that the present disclosure is not entitled to antedate such publication by virtue of prior invention.

### 2. ABBREVIATIONS

GA glucoamylase
GAU glucoamylase unit
wt weight percent
° C. degrees Centigrade
rpm revolutions per minute
aa or AA amino acid
bp base pair
kb kilobase pair
kD kilodaltons
g or gm grams
μg micrograms
mg milligrams
μl and μL microliters
ml and mL milliliters
mm millimeters
μm micrometer
M molar
mM millimolar
μM micromolar
U units
V volts
MW molecular weight
sec(s) or s(s) second/seconds
min(s) or m(s) minute/minutes
hr(s) or h(s) hour/hours
DO dissolved oxygen

### ABS Absorbance

EtOH ethanol
PSS physiological salt solution
m/v mass/volume
MTP microtiter plate

### N Normal

DP1 monosaccharides
DP2 disaccharides
DP>3 oligosaccharides, sugars having a degree of polymerization greater than 3
ppm parts per million
SBD starch binding domain
CD catalytic domain
PCR polymerase chain reaction
WT wild-type

### RDF Real Degree of Attenuation

SG Specific gravity

### PU Pasteurisation Units

MkGAI Monascus kaoliang glucoamylase I
MkGAII Monascus kaoliang glucoamylase II
H. jecorina Hypocrea jecorina
T. reesei Trichoderma reesei
TrGA Trichoderma reesei glucoamylse
AnGA Aspergillus Niger glucoamylase

### 3. GLUCOAMYLASE POLYPEPTIDES

### Parent Glucoamylases

In some embodiments, the present disclosure provides a glucoamylase variant. The glucoamylase variant is a variant of a parent glucoamylase, which may comprise both a catalytic domain and a starch binding domain. In some embodiments, the parent glucoamylase comprises a catalytic domain having an amino acid sequence as illustrated in SEQ ID NO: 1, 2, 3, 5, 6, 7, 8, 9 or 13 or having an amino acid sequence displaying at least about 80%, about 85%, about 90%, about 95%, about 97%, about 99%, or about 99.5% sequence identity with one or more of the amino acid sequences illustrated in SEQ ID NO:1, 2, 3, 5, 6, 7, 8, 9 or 13. In yet other embodiments, the parent glucoamylase comprises a catalytic domain encoded by a DNA sequence that hybridizes under medium, high, or stringent conditions with a DNA encoding the catalytic domain of a glucoamylase having one of the amino acid sequences of SEQ ID NO: 1, 2 or 3.

In one aspect, a variant as described herein has at the most 480, 481, 482, 483, 484, 485, 486, 487, 488, 489, 490, 495, 500, 505, 507, 515, 525, 535, 545, 555, 565 or 573 amino acid residues.

In one aspect, a variant as described herein has at the most 15, 14, 13, 12, 11, 10, 9, 8, 7, 6, 5, 4, 3, 2 or 1 amino acid residue substitutions.

In one aspect, a variant as described herein has at the most a deletion with a length of 20, 19, 18, 17, 16, 15, 14, 13, 12, 11, 10, 9, 8, 7, 6, 5, 4, 3, 2 or 1 amino acid residues.

In one aspect, a variant as described herein has at the most an insertion with a length of 20, 19, 18, 17, 16, 15, 14, 13, 12, 11, 10, 9, 8, 7, 6, 5, 4, 3, 2 or 1 amino acid residues.

In some embodiments, the parent glucoamylase comprises a starch binding domain having an amino acid sequence as illustrated in SEQ ID NO 1, 2, 11, 24, 25, 26, 27, 28, or 29, or having an amino acid sequence displaying at least about 80%, about 85%, about 90%, about 95%, about 97%, about 99%, or about 99.5% sequence identity with one or more of the amino acid sequence illustrated in SEQ ID NO 1, 2, 11, 24, 25, 26, 27, 28, or 29. In yet other embodiments, the parent glucoamylase comprises a starch binding domain encoded by a DNA sequence that hybridizes under medium, high, or stringent conditions with a DNA encoding the starch binding domain of a glucoamylase having one of the amino acid sequences of SEQ ID NO: 1, 2, or 11.

Predicted structure and known sequences of glucoamylases are conserved among fungal species (Coutinho et al., 1994, Protein Eng., 7:393-400 and Coutinho et al., 1994, Protein Eng., 7: 749-760). In some embodiments, the parent glucoamylase is a filamentous fungal glucoamylase. In some embodiments, the parent glucoamylase is obtained from a Trichoderma strain (e.g., T. reesei, T. longibrachiatum, T. strictipilis, T. asperellum, T. konilangbra and T. hazianum), an Aspergillus strain (e.g. A. niger, A. nidulans, A. kawachi, A. awamori and A. orzyae), a Talaromyces strain (e.g. T. emersonii, T. thermophilus, and T. duponti), a Hypocrea strain (e.g. H. gelatinosa, H. orientalis, H. vinosa, and H. citrina), a Fusarium strain (e.g., F. oxysporum, F. roseum, and F. venenatum), a Neurospora strain (e.g., N. crassa) and a Humicola strain (e.g., H. grisea, H. insolens and H. lanuginose), a Penicillium strain (e.g., P. notatum or P. chrysogenum), or a Saccharomycopsis strain (e.g., S. fibuligera).

In some embodiments, the parent glucoamylase may be a bacterial glucoamylase. For example, the polypeptide may be obtained from a gram-positive bacterial strain such as Bacillus (e.g., B. alkalophilus, B. amyloliquefaciens, B. lentus, B. licheniformis, B. stearothermophilus, B. subtilis and B. thuringiensis) or a Streptomyces strain (e.g., S. lividans).

In some embodiments, the parent glucoamylase will comprise a catalytic domain having at least about 80%, about 85%, about 90%, about 93%, about 95%, about 97%, about 98%, or about 99% sequence identity with the catalytic domain of the TrGA amino acid sequence of SEQ ID NO: 3.

In other embodiments, the parent glucoamylase will comprise a catalytic domain having at least about 90%, about 93%, about 95%, about 96%, about 97%, about 98%, or about 99% sequence identity with the catalytic domain of the Aspergillus parent glucoamylase of SEQ ID NO: 5 or SEQ ID NO: 6.

In yet other embodiments, the parent glucoamylase will comprise a catalytic domain having at least about 90%, about 95%, about 97%, or about 99% sequence identity with the catalytic domain of the Humicola grisea (HgGA) parent glucoamylase of SEQ ID NO: 8.

In some embodiments, the parent glucoamylase will comprise a starch binding domain having at least about 80%, about 85%, about 90%, about 95%, about 97%, or about 98% sequence identity with the starch binding domain of the TrGA amino acid sequence of SEQ ID NO: 1, 2, or 11.

In other embodiments, the parent glucoamylase will comprise a starch binding domain having at least about 90%, about 95%, about 97%, or about 99% sequence identity with the catalytic domain of the Humicola grisea (HgGA) glucoamylase of SEQ ID NO: 24.

In other embodiments, the parent glucoamylase will comprise a starch binding domain having at least about 90%, about 95%, about 97%, or about 99% sequence identity with the catalytic domain of the Thielavia terrestris (TtGA) glucoamylase of SEQ ID NO: 29 see also alignment in FIGS. 10D and 10E.

In other embodiments, the parent glucoamylase will comprise a starch binding domain having at least about 90%, about 95%, about 97%, or about 99% sequence identity with the catalytic domain of the Thermomyces lanuginosus (ThGA) glucoamylase of SEQ ID NO: 25 (FIGS. 10D and 10E).

In other embodiments, the parent glucoamylase will comprise a starch binding domain having at least about 90%, about 95%, about 97%, or about 99% sequence identity with the catalytic domain of the Talaromyces emersoniit (TeGA) glucoamylase of SEQ ID NO: 26.

In yet other embodiments, the parent glucoamylase will comprise a starch binding domain having at least about 90%, about 93%, about 95%, about 96%, about 97%, about 98%, or about 99% sequence identity with the starch binding domain of the Aspergillus parent glucoamylase of SEQ ID NO: 27 or 28.

In some embodiments, the parent glucoamylase will have at least about 80%, about 85%, about 88%, about 90%, about 93%, about 95%, about 96%, about 97%, about 98%, or about 99% sequence identity with the TrGA amino acid sequence of SEQ ID NO: 1 or 2.

In further embodiments, a Trichoderma glucoamylase homologue will be obtained from a Trichoderma or Hypocrea strain. Some typical Trichoderma glucoamylase homologues are described in U.S. Pat. No. 7,413,887 and reference is made specifically to amino acid sequences set forth in SEQ ID NOs: 17-22 and 43-47 of the reference.

In some embodiments, the parent glucoamylase is TrGA comprising the amino acid sequence of SEQ ID NO: 2, or a Trichoderma glucoamylase homologue having at least about 80%, about 85%, about 88%, about 90%, about 93%, about 95%, about 96%, about 97%, about 98%, or about 99% sequence identity to the TrGA sequence (SEQ ID NO: 2).

A parent glucoamylase can be isolated and/or identified using standard recombinant DNA techniques. Any standard techniques can be used that are known to the skilled artisan. For example, probes and/or primers specific for conserved regions of the glucoamylase can be used to identify homologs in bacterial or fungal cells (the catalytic domain, the active site, etc.). Alternatively, degenerate PCR can be used to identify homologues in bacterial or fungal cells. In some cases, known sequences, such as in a database, can be analyzed for sequence and/or structural identity to one of the known glucoamylases, including SEQ ID NO: 2, or a known starch binding domains, including SEQ ID NO: 11. Functional assays can also be used to identify glucoamylase activity in a bacterial or fungal cell. Proteins having glucoamylase activity can be isolated and reverse sequenced to isolate the corresponding DNA sequence. Such methods are known to the skilled artisan.

### Glucoamylase Structural Homology

The central dogma of molecular biology is that the sequence of DNA encoding a gene for a particular enzyme, determines the amino acid sequence of the protein, this sequence in turn determines the three-dimensional folding of the enzyme. This folding brings together disparate residues that create a catalytic center and substrate binding surface and this results in the high specificity and activity of the enzymes in question.

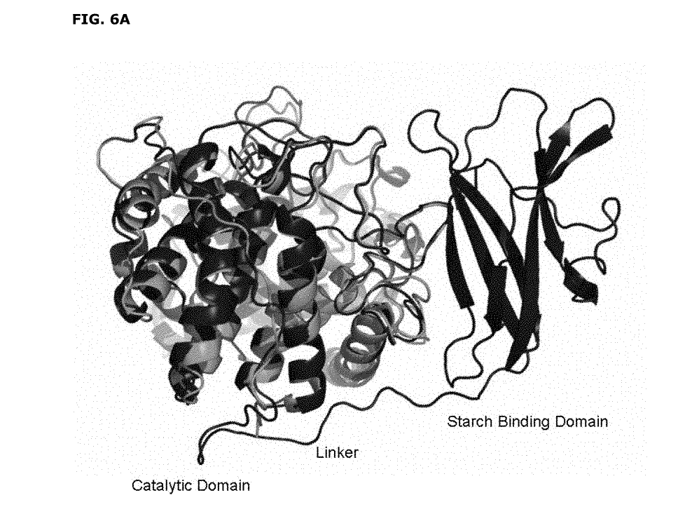

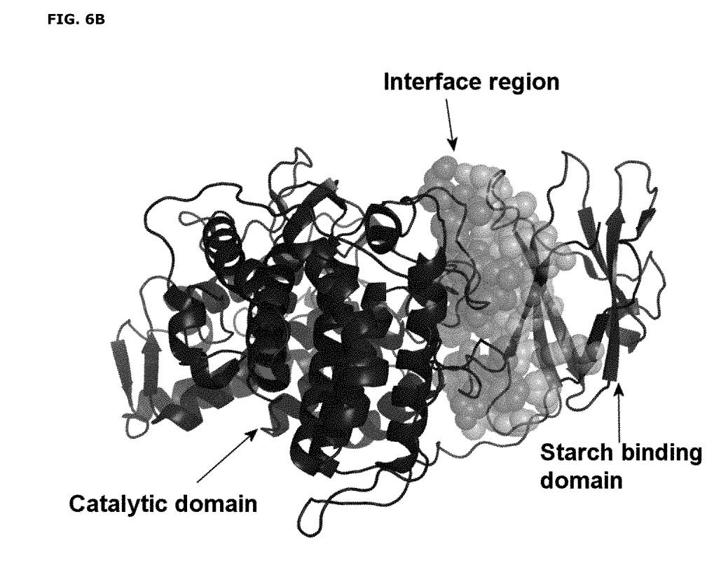

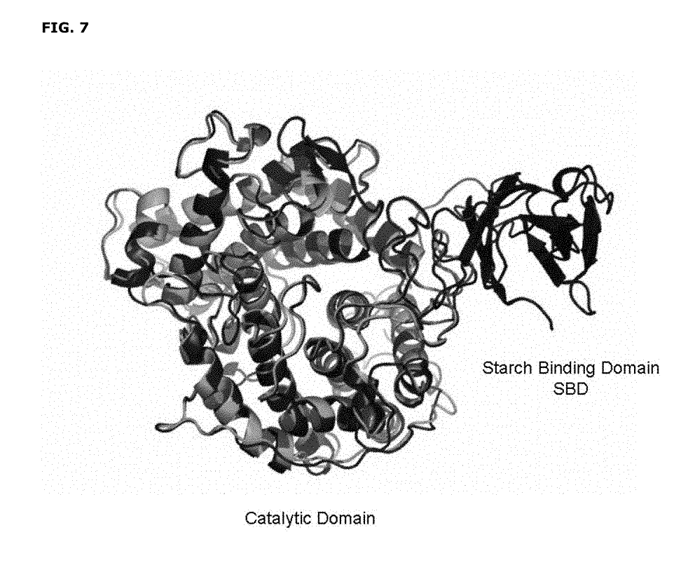

Glucoamylases consist of as many as three distinct structural domains, a catalytic domain of approximately 450 residues that is structurally conserved in all glucoamylases, generally followed by a linker region consisting of between 30 and 80 residues that are connected to a starch binding domain of approximately 100 residues. The structure of the Trichoderma reesei glucoamylase with all three regions intact was determined to 1.8 Angstrom resolution herein (see Table 20 in WO2009/067218 (Danisco US Inc., Genencor Division) page 94-216 incorporated herein by reference and Example 11 in WO2009/067218 (Danisco US Inc., Genencor Division) page 89-93 incorporated herein by reference). Using the coordinates (see Table 20 in WO2009/067218 (Danisco US Inc., Genencor Division) page 94-216 incorporated herein by reference), the structure was aligned with the coordinates of the catalytic domain of the glucoamylase from Aspergillus awamori strain X100 that was determined previously (Aleshin, A. E., Hoffman, C., Firsov, L. M., and Honzatko, R. B. Refined crystal structures of glucoamylase from Aspergillus awamori var. X100. J. Mol. Biol. 238: 575-591 (1994)). The Aspergillus awamori crystal structure only included the catalytic domain. As seen in FIGS. 6A and 7, the structure of the catalytic domains overlap very closely, and it is possible to identify equivalent residues based on this structural superposition. It is believed that all glucoamylases share the basic structure depicted in FIGS. 6A and 7.

The catalytic domain of TrGA thus has approximately 450 residues such as residues 1-453 of TrGA SEQ ID NO:2 and is a twelve helix double barrel domain. The helices and loops of the catalytic domain can be defined in terms of the residues of TrGA with SEQ ID NO:2 forming them:

helix 1 residues 2-20,
loop 1 residues 21-51,
helix 2 residues 52-68,
loop 2 residues 69-71,
helix 3 residues 72-90,
loop 3 residues 91-125,
helix 4 residues 126-145,
loop 4 residues 146,
helix 5 residues 147-169,
helix 6 residues 186-206,
loop 6 residues 207-210,
helix 7 residues 211-227,
loop 7 residues 211-227,
helix 8 residues 250-275,
loop 8 residues 260-275,
helix 9 residues 276-292,
helix 10 residues 322-342,
loop 10 residues 343-371,
helix 11 residues 372-395,
loop 11 residues 396-420,
helix 12 residues 421-434,
loop 12 residues 435-443,
helix 13 residues 444-447,
loop 13 residues 448-453

The linker domain has between 30 and 80 residues such as residues 454-490 of TrGA with SEQ ID NO: 2.

The starch binding domain of TrGA has approximately 100 residues such as residues 496-596 of TrGA with SEQ ID NO:2 consisting of the beta sandwich composed of two twisted three stranded sheets. The sheets, helices and loops of the starch binding domain can be defined in terms of the residues of TrGA with SEQ ID NO:2 forming them:

sheet 1′ residues 496-504,
loop 1′ residues 505-511,
sheet 2′ residues 512-517,
interconnecting loop 2′ residues 518-543,
sheet 3′ residues 544-552,
loop 3′ residues 553,
sheet 4′ residues 554-565,
loop 4′ residues 566-567,
sheet 5′ residues 568-572,
inter-sheet segment residues 573-577,
sheet 5a′ residues 578-582,
loop 5′ residues 583-589,
sheet 6′ residues 590-596,

The positioning of the catalytic domain in TrGA against the surface of the stach binding domain leaves a interface region in between the two domains. This connecting surface area corresponds to the following positions in TrGA (SEQ ID NO 2): 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596. The position of these residues in the three dimensional structure of TrGA are shown in FIG. 6B.

It is possible to identify equivalent residues based on structural superposition in other glucoamylases as described in further detail below.

FIG. 6A is a comparison of the three dimensional structures of the Trichoderma reesei glucoamylase (black) of SEQ ID NO: 2 and of Aspergillus awamorii glucoamylase (grey) viewed from the side. In this view, the relationship between the catalytic domain and the linker region and the starch binding domain can be seen.

FIG. 6B depicts the three dimensional structure of Trichoderma reesei glucoamylase (black) (SEQ ID NO: 2) viewed from the side with residues forming the interface region in between the catalytic domain and the starch binding domain highlighted (residues from the catalytic domain in dark gray and residues from the starch binding domain in light gray).

FIG. 7 is a comparison of the three dimensional structures of the Trichoderma reesei glucoamylase (black) of SEQ ID NO: 2 and of Aspergillus awamorii glucoamylase (grey) viewed from the top. The glucoamylases shown here and indeed all known glucoamylases to date share this structural homology. The conservation of structure correlates with the conservation of activity and a conserved mechanism of action for all glucoamylases. Given this high homology, changes resulting from site specific variants of the Trichoderma glucoamylase resulting in altered functions would also have similar structural and therefore functional consequences in other glucoamylases. Therefore, the teachings of which variants result in desirable benefits can be applied to other glucoamylases.

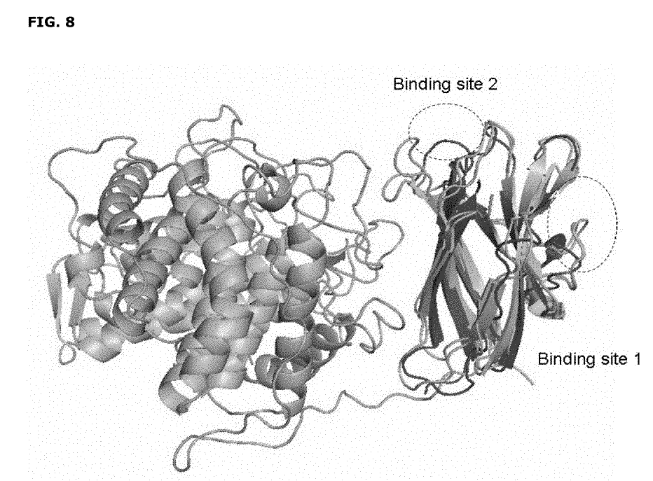

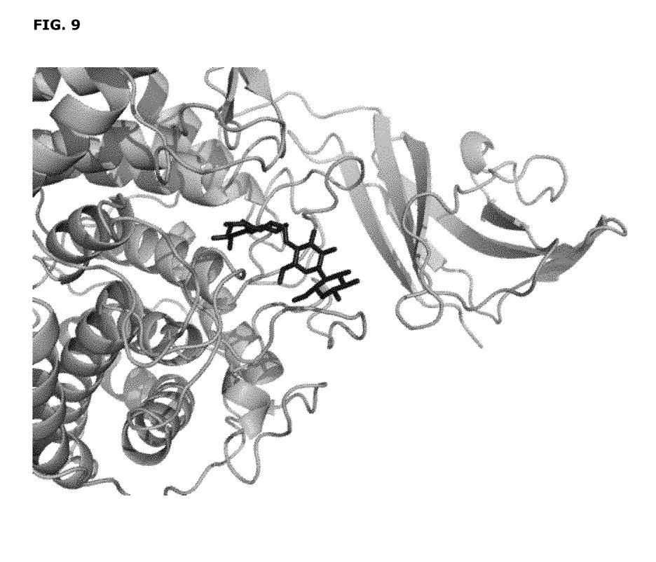

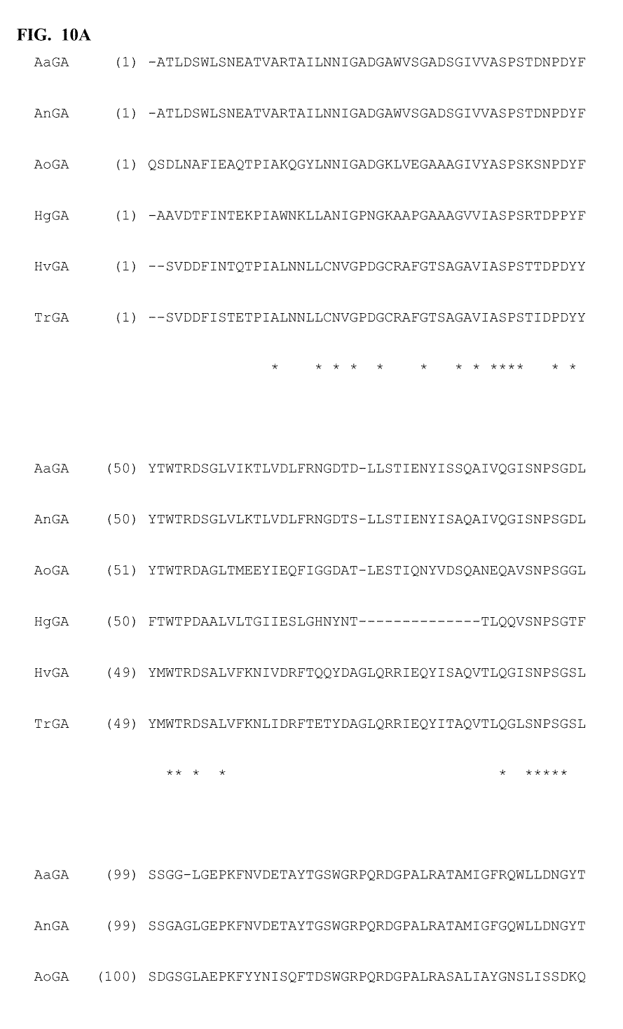

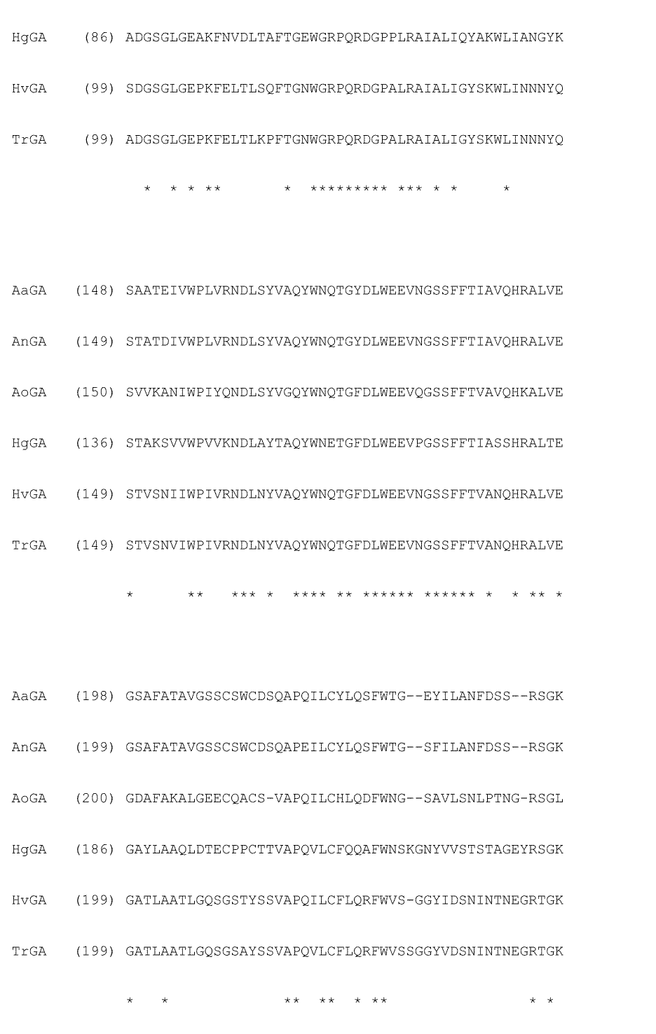

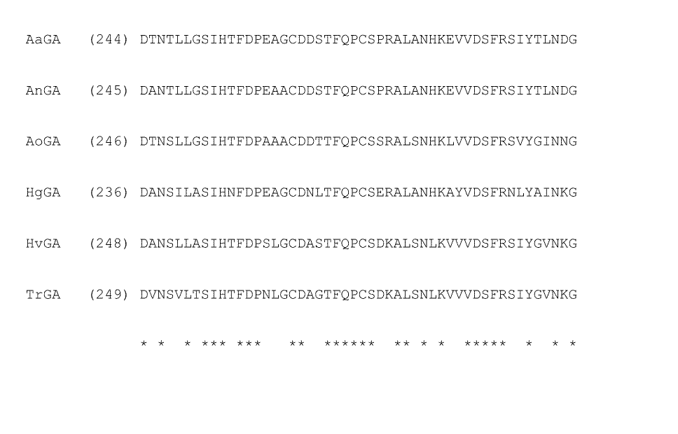

A further crystal structure was produced using the coordinates in Table 20 in WO2009/067218 (Danisco US Inc., Genencor Division) page 94-216 incorporated herein by reference for the Starch Binding Domain (SBD). The SBD for TrGA was aligned with the SBD for A. niger. As shown in FIG. 8, the structure of the A. niger and TrGA SBDs overlaps very closely. It is believed that while all starch binding domains share at least some of the basic structure depicted in FIG. 8, some SBDs are more structurally similar than others. For example, the TrGA SBD can be classified as within the carbohydrate binding module 20 family within the CAZY database (cazy.org). The CAZY database describes the families of structurally-related catalytic and carbohydrate-binding modules (or functional domains) of enzymes that degrade, modify, or create glycosidic bonds. Given a high structural homology, site specific variants of the TrGA SBD resulting in altered function would also have similar structural and therefore functional consequences in other glucoamylases having SBDs with similar structure to that of the TrGA SBD, particularly those classified within the carbohydrate binding module 20 family. Thus, the teachings of which variants result in desirable benefits can be applied to other SBDs having structural similarity.

Thus, the amino acid position numbers discussed herein refer to those assigned to the mature Trichoderma reesei glucoamylase sequence having SEQ ID NO: 2. The present disclosure, however, is not limited to the variants of Trichoderma glucoamylase, but extends to glucoamylases containing amino acid residues at positions that are “equivalent” to the particular identified residues in Trichoderma reesei glucoamylase (SEQ ID NO: 2). In some embodiments of the present disclosure, the parent glucoamylase is a Talaromyces GA and the substitutions are made at the equivalent amino acid residue positions in Talaromyces glucoamylase (see e.g., SEQ ID NO: 23) as those described herein. In other embodiments, the parent glucoamylase comprises SEQ ID NOs: 1, 2, 13, 18, 19, 20, 21, and 22.

“Structural identity” determines whether the amino acid residues are equivalent. Structural identity is a one-to-one topological equivalent when the two structures (three dimensional and amino acid structures) are aligned. A residue (amino acid) position of a glucoamylase is “equivalent” to a residue of T. reesei glucoamylase if it is either homologous (i.e., corresponding in position in either primary or tertiary structure) or analogous to a specific residue or portion of that residue in T. reesei glucoamylase (having the same or similar functional capacity to combine, react, or interact chemically).

In order to establish identity to the primary structure, the amino acid sequence of a glucoamylase can be directly compared to Trichoderma reesei glucoamylase primary sequence and particularly to a set of residues known to be invariant in glucoamylases for which sequence is known. For example, FIGS. 10A and 10B herein show the conserved residues between glucoamylases. FIGS. 10D and 10E show an alignment of starch binding domains from various glucoamylases. After aligning the conserved residues, allowing for necessary insertions and deletions in order to maintain alignment (i.e. avoiding the elimination of conserved residues through arbitrary deletion and insertion), the residues equivalent to particular amino acids in the primary sequence of Trichoderma reesei glucoamylase are defined. Alignment of conserved residues typically should conserve 100% of such residues. However, alignment of greater than about 75% or as little as about 50% of conserved residues is also adequate to define equivalent residues. Further, the structural identity can be used in combination with the sequence identity to identify equivalent residues.

For example, in FIGS. 10A and 10B, the catalytic domains of glucoamylases from six organisms are aligned to provide the maximum amount of homology between amino acid sequences. A comparison of these sequences shows that there are a number of conserved residues contained in each sequence as designated by an asterisk. These conserved residues, thus, may be used to define the corresponding equivalent amino acid residues of Trichoderma reesei glucoamylase in other glucoamylases such as glucoamylase from Aspergillus niger. Similarly, FIGS. 10D and 10E show the starch binding domains of glucoamylases from seven organisms aligned to identify equivalent residues.

Structural identity involves the identification of equivalent residues between the two structures. “Equivalent residues” can be defined by determining homology at the level of tertiary structure (structural identity) for an enzyme whose tertiary structure has been determined by X-ray crystallography. Equivalent residues are defined as those for which the atomic coordinates of two or more of the main chain atoms of a particular amino acid residue of the Trichoderma reesei glucoamylase (N on N, CA on CA, C on C and 0 on 0) are within 0.13 nm and optionally 0.1 nm after alignment. In one aspect, at least 2 or 3 of the four possible main chain atoms are within 0.1 nm after alignment. Alignment is achieved after the best model has been oriented and positioned to give the maximum overlap of atomic coordinates of non-hydrogen protein atoms of the glucoamylase in question to the Trichoderma reesei glucoamylase. The best model is the crystallographic model giving the lowest R factor for experimental diffraction data at the highest resolution available.

$$R factor = \frac{{\sum }_{h} \vert Fo ( h ) \vert - \vert Fc ( h ) \vert}{{\sum }_{h} \vert Fo ( h ) \vert}$$

Equivalent residues that are functionally analogous to a specific residue of Trichoderma reesei glucoamylase are defined as those amino acids of the enzyme that may adopt a conformation such that they either alter, modify or contribute to protein structure, substrate binding or catalysis in a manner defined and attributed to a specific residue of the Trichoderma reesei glucoamylase. Further, they are those residues of the enzyme (for which a tertiary structure has been obtained by X-ray crystallography) that occupy an analogous position to the extent that, although the main chain atoms of the given residue may not satisfy the criteria of equivalence on the basis of occupying a homologous position, the atomic coordinates of at least two of the side chain atoms of the residue lie with 0.13 nm of the corresponding side chain atoms of Trichoderma reesei glucoamylase. The coordinates of the three dimensional structure of Trichoderma reesei glucoamylase are set forth in Table 20 in WO2009/067218 (Danisco US Inc., Genencor Division) page 94-216 incorporated herein by reference and can be used as outlined above to determine equivalent residues on the level of tertiary structure.

Some of the residues identified for substitution are conserved residues whereas others are not. In the case of residues that are not conserved, the substitution of one or more amino acids is limited to substitutions that produce a variant that has an amino acid sequence that does not correspond to one found in nature. In the case of conserved residues, such substitutions should not result in a naturally-occurring sequence.

### Glucoamylase Variants

The variants according to the disclosure include at least one substitution, deletion or insertion in the amino acid sequence of a parent glucoamylase that makes the variant different in sequence from a parent glucoamylase. In some embodiments, the variants of the disclosure will have at least about 20%, about 40%, about 50%, about 60%, about 70%, about 80%, about 85%, about 90%, about 95%, about 97%, or about 100% of the glucoamylase activity as that of the TrGA (SEQ ID NO: 2), a parent glucoamylase that has at least 80% sequence identity to TrGA (SEQ ID NO: 2). In some embodiments, the variants according to the disclosure will comprise a substitution, deletion or insertion in at least one amino acid position of the parent TrGA (SEQ ID NO: 2), or in an equivalent position in the sequence of another parent glucoamylase having at least about 80%, about 85%, about 90%, about 95%, about 97%, about 98%, or about 99% sequence identity to the TrGA sequence (SEQ ID NO: 2).

In other embodiments, the variant according to the disclosure will comprise a substitution, deletion or insertion in at least one amino acid position of a fragment of the parent TrGA, wherein the fragment comprises the catalytic domain of the TrGA sequence (SEQ ID NO: 3) or in an equivalent position in a fragment comprising the catalytic domain of a parent glucoamylase having at least about 80%, about 85%, about 90%, about 95%, about 97%, about 98%, or about 99% sequence identity to the catalytic-domain-containing fragment of the SEQ ID NO: 3, 5, 6, 7, 8, or 9. In some embodiments, the fragment will comprise at least about 400, about 425, about 450, or about 500 amino acid residues of TrGA catalytic domain (SEQ ID NO: 3).

In other embodiments, the variant according to the disclosure will comprise a substitution, deletion or insertion in at least one amino acid position of a fragment of the parent TrGA, wherein the fragment comprises the starch binding domain of the TrGA sequence (SEQ ID NO: 11) or in an equivalent position in a fragment comprising the starch binding domain of a parent glucoamylase having at least about 80%, about 85%, about 90%, about 95%, about 97%, about 98%, or about 99% sequence identity to the starch-binding-domain-containing fragment of SEQ ID NO: 11, 24, 25, 26, 27, 28, and/or 29. In some embodiments, the fragment will comprise at least about 40, about 50, about 60, about 70, about 80, about 90, about 100, or about 109 amino acid residues of TrGA starch binding domain (SEQ ID NO: 11).

In some embodiments, when the parent glucoamylase includes a catalytic domain, a linker region, and a starch binding domain, the variant will comprise a substitution, deletion or insertion in at least one amino acid position of a fragment comprising part of the linker region. In some embodiments, the variant will comprise a substitution, deletion, or insertion in the amino acid sequence of a fragment of the TrGA sequence (SEQ ID NO: 2).

Structural identity with reference to an amino acid substitution means that the substitution occurs at the equivalent amino acid position in the homologous glucoamylase or parent glucoamylase. The term equivalent position means a position that is common to two parent sequences that is based on an alignment of the amino acid sequence of the parent glucoamylase in question as well as alignment of the three-dimensional structure of the parent glucoamylase in question with the TrGA reference glucoamylase amino acid sequence and three-dimensional sequence. For example, with reference to FIG. 10A, position 24 in TrGA (SEQ ID NO: 2 or 3) is D24 and the equivalent position for Aspergillus niger (SEQ ID NO: 6) is position D25, and the equivalent position for Aspergillus oryzea (SEQ ID NO: 7) is position D26. See FIGS. 6A and 7 for an exemplary alignment of the three-dimensional sequence.

Accordingly, in one aspect, a glucoamylase variant is described, which glucoamylase variant when in its crystal form has a crystal structure for which the atomic coordinates of the main chain atoms have a root-mean-square deviation from the atomic coordinates of the equivalent main chain atoms of TrGA (as defined in Table 20 in WO2009/067218) of less than 0.13 nm following alignment of equivalent main chain atoms, and which have a linker region, a starch binding domain and a catalytic domain, said variant comprising two or more amino acid substitutions relative to the amino acid sequence of the parent glucoamylase in interconnecting loop 2′ of the starch binding domain, and/or in loop 1, and/or in helix 2, and/or in loop 11, and/or in helix 12 of the catalytic domain. In a further aspect, the root-mean-square deviation from the atomic coordinates of the equivalent main chain atoms of TrGA (as defined in Table 20 in WO2009/067218) is less than 0.12 nm, such as less than 0.11 or such as less than 0.10.

In a further aspect, the glucoamylase variant has a starch binding domain that has at least 96%, 97%, 98%, 99%, or 99.5% sequence identity with the starch binding domain of SEQ ID NO: 1, 2, 11, 13, 24, 25, 26, 27, 28, or 29. In a further aspect, the glucoamylase variant has a catalytic domain that has at least 80%, 85%, 90%, 95%, or 99.5% sequence identity with the catalytic domain of SEQ ID NO: 1, 2, 3, 5, 6, 7, 8, 9 or 13.

In a further aspect, the parent glucoamylase is a fungal glucoamylase.

In a further aspect, the parent glucoamylase is selected from a glucoamylase obtained from a Trichoderma spp., an Aspergillus spp., a Humicola spp., a Penicillium spp., a Talaromycese spp., or a Schizosaccharmyces spp.

In a further aspect, the parent glucoamylase is obtained from a Trichoderma spp. or an Aspergillus spp.

In a further aspect, the glucoamylase has been purified. The glucoamylases of the present disclosure may be recovered or purified from culture media by a variety of procedures known in the art including centrifugation, filtration, extraction, precipitation and the like.

In some embodiments, the glucoamylase variant will include at least two substitutions in the amino acid sequence of a parent. In some embodiments, the glucoamylase variant will include at least two, three or four substitutions in the amino acid sequence of a parent such as SEQ ID NO: 2 or 13. In some embodiments, the glucoamylase variant will include at the most two, three or four substitutions in the amino acid sequence of a parent such as SEQ ID NO: 2 or 13. In further embodiments, the variant may have more than two substitutions. For example, the variant may have 2, 3, 4, 5, 6, 7, 8, 9, 10, 15, 20, or 25 amino acid substitutions, deletions, or insertions as compared to a corresponding parent glucoamylase.

In some embodiments, a glucoamylase variant comprises a substitution, deletion or insertion, and typically a substitution in at least one amino acid position in a position corresponding to the regions of non-conserved amino acids as illustrated in FIGS. 10A, 10B, 10D, and 10E (e.g., amino acid positions corresponding to those positions that are not designated by “*” in FIGS. 10A, 10B, 10D, and 10E).

In some embodiments, the parent glucoamylase will have at least about 50%, about 60%, about 70%, about 80%, about 90%, about 95%, about 96%, about 97%, about 98%, or about 99% sequence identity with SEQ ID NO: 2 or SEQ ID NO: 13. In other embodiments, the parent glucoamylase will be a Trichoderma glucoamylase homologue. In some embodiments, the variant will have altered properties. In some embodiments, the parent glucoamylase will have structural identity with the glucoamylase of SEQ ID NO: 2 or SEQ ID NO: 13.

In some embodiments, the glucoamylase variant may differ from the parent glucoamylase only at the specified positions.

The parent glucoamylase may comprise a starch binding domain that has at least 95% sequence identity with SEQ ID NO: 1, 2, 11, 13, 24, 25, 26, 27, 28, or 29. The parent glucoamylase may have at least 80% sequence identity with SEQ ID NO: 1 or 2; for example it may comprise SEQ ID NO: 1 or 2. Optionally the parent glucoamylase may consist of SEQ ID NO: 1, 2 or 13.

Glucoamylase variants of the disclosure may also include chimeric or hybrid glucoamylases with, for example a starch binding domain (SBD) from one glucoamylase and a catalytic domain and linker from another. For example, a hybrid glucoamylase can be made by swapping the SBD from AnGA (SEQ ID NO: 6) with the SBD from TrGA (SEQ ID NO: 2), making a hybrid with the AnGA SBD and the TrGA catalytic domain and linker. Alternatively, the SBD and linker from AnGA can be swapped for the SBD and linker of TrGA.

In some aspects, the variant glucoamylase exhibits altered thermostability as compared to the parent glucoamylase. In some aspects, the altered thermostability may be decreased thermostability as compared to the parent glucoamylase. In some embodiments, the altered property is altered specific activity compared to the parent glucoamylase. In some embodiments, the specific activity may be similar or increased compared to the parent glucoamylase. In some embodiments, the altered property is decreased thermostability at lower temperatures as compared to the parent glucoamylase. In some embodiments, the altered property is both similar or increased specific activity and decreased thermostability as compared to the parent glucoamylase.

A number of parent glucoamylases have been aligned with the amino acid sequence of TrGA. FIGS. 10A and 10B includes the catalytic domain of the following parent glucoamylases Aspergillus awamori (AaGA) (SEQ ID NO: 5); Aspergillus niger (AnGA) (SEQ ID NO: 6); Aspergillus orzyae (AoGA) (SEQ ID NO: 7); Humicola grisea (HgGA) (SEQ ID NO: 8); and Hypocrea vinosa (HvGA) (SEQ ID NO: 9). The % identity of the catalytic domains is represented in Table A below.

TABLE A
Sequence homology between various fungal glucoamylases

| | AaGA | AnGA | AoGA | HgGA | HvGA | TrGA |
|---|---|---|---|---|---|---|
| AaGA | 100 | 95 | 58 | 53 | 57 | 56 |
| AnGA | | 100 | 59 | 53 | 57 | 56 |
| AoGA | | | 100 | 55 | 56 | 56 |
| HgGA | | | | 100 | 61 | 63 |
| HvGA | | | | | 100 | 91 |
| TrGA | | | | | | 100 |

In some embodiments, for example, the variant glucoamylase will be derived from a parent glucoamylase that is an Aspergillus glucoamylase, a Humicola glucoamylase, or a Hypocrea glucoamylase.

In one aspect, the variant as contemplated herein is obtained by recombinant expression in a host cell.

In one aspect, the variant described herein has a glucoamylase activity (GAU) of at least 0.05 GAU/mg, 0.1 GAU/mg, 0.2 GAU/mg, 0.3 GAU/mg, 0.4 GAU/mg, 0.5 GAU/mg, 0.6 GAU/mg, 0.7 GAU/mg, 0.8 GAU/mg, 0.9 GAU/mg, 1 GAU/mg, 2 GAU/mg, 3 GAU/mg, 5 GAU/mg, or 10 GAU/mg.

In another aspect, the variant described herein has a glucoamylase activity (GAU) of 0.05-10 GAU/mg, such as 0.1-5 GAU/mg, such as 0.5-4 GAU/mg, such as 0.7-4 GAU/mg, such as 2-4 GAU/mg.

In yet a further aspect, the glucoamylase variants described herein comprises or consist of the variant of SEQ ID NO:14, 15 or 16.

### Characterization of Variant Glucoamylases

The present disclosure also provides glucoamylase variants having at least one altered property (e.g., improved property) as compared to a parent glucoamylase and particularly to the TrGA. In some embodiments, at least one altered property (e.g., improved property) is selected from the group consisting of GAU activity, real degree of fermentation, expression level, thermal stability and specific activity. Typically, the altered property is reduced thermal stability, enhanced real degree of fermentation and/or increased specific activity. The reduced thermal stability typically is at higher temperatures.

The glucoamylase variants of the disclosure may also provide higher rates of starch hydrolysis at low substrate concentrations as compared to the parent glucoamylase. The variant may have a higher Vₘₐₓ or lower Kₘ than a parent glucoamylase when tested under the same conditions. For example the variant glucoamylase may have a higher Vₘₐₓ at a temperature range of about 25° C. to about 40° C. (e.g., about 25° C. to about 35° C.; about 30° C. to about 35° C.). The Michaelis-Menten constant, Kₘ and Vₘₐₓ values can be easily determined using standard known procedures. In another aspect, the glucoamylase may also exhibit a reduced starch hydrolysis activity which is not more than 5%, not more than 10% or not more than 15% reduced as compared to the parent glucoamylase such as TrGA or TrGA CS4.

Variant Glucoamylases with Altered Thermostability

In some aspects, the disclosure relates to a variant glucoamylase having altered thermal stability as compared to a parent (wild-type). Altered thermostability can be at increased temperatures or at decreased temperatures. Thermostability is measured as the % residual activity after incubation for up to 100 sec at 72° C. in NaAc buffer pH 4.5 or regular pilsner beer, TrGA has a residual activity of 24% as compared to the initial activity before incubation at these conditions. The residual activity of TrGA under these conditions are comparable to incubation of the enzyme for 1 hour at 64° C. in NaAc buffer pH 4.5 that leaves about 15% residual activity as compared to the initial activity before incubation. Thus, in some embodiments, variants with decreased thermostability (i.e. more thermolabile) have a residual activity that is between at least about 50% and at least about 100% less than that of the parent (after incubation for 100 sec at 72° C. in regular pilsner beer pH 4.5), including about 51%, about 52% about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 100% as compared to the initial activity before incubation. For example, when the parent residual activity is 24%, a variant with decreased thermal stability may have a residual activity of between about 2% and about 3%. In some embodiments, the glucoamylase variant will have decreased thermostability such as retaining at least about 0%, about 1%, about 2%, about 3%, about 4%, about 5%, about 6%, about 7%, about 8%, about 9%, about 10%, about 15%, or about 20% enzymatic activity after exposure to altered temperatures over a given time period, for example, at least about 50 sec, about 60 sec, about 70 sec, about 100 sec, or about 150 at 72° C. In some embodiments, the variant has decreased thermal stability compared to the parent glucoamylase at selected temperatures in the range of about 40° C. to about 80° C., also in the range of about 50° C. to about 75° C., and in the range of about 60° C. to about 70° C., and at a pH range of about 4.0 to about 6.0. In some embodiments, the thermostability is determined as described in the Assays and Methods. That method may be adapted as appropriate to measure thermostability at other temperatures. Alternatively the thermostability may be determined at 64° C. as described there. In some embodiments, the variant has decreased thermal stability at lower temperature compared to the parent glucoamylase at selected temperature in the range of about 20° C. to about 50° C., including about 35° C. to about 45° C. and about 30° C. to about 40° C.

In some embodiments, variants having a decreased thermostability include one or more deletions, substitutions or insertions and particularly substitutions in the following positions in the amino acid sequence set forth in SEQ ID NO: 2: one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase.

In some embodiments, variants having a decreased thermostability include one or more deletions, substitutions or insertions, and particularly substitutions in the following positions in the amino acid sequence set forth in SEQ ID NO: 2 or 13: one or two amino acid substitutions in the group of interface amino acids consisting of residues 29, 43, 48, 116, and 502 of SEQ ID NO: 2 or 13; and one, two or three amino acid substitutions in the group of catalytic core amino acid residues consisting of residues 97, 98, 147, 175, 483 and 484 of SEQ ID NO: 2 or 13. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO: 2 or 13. In some embodiments, the parent glucoamylase will also have structural identity to SEQ ID NO: 2. In some embodiments, the variant having decreased thermostability has one or two of the following substitutions: F29V, F29Q, I43Q, Y48V, F116M, H502S, H502E, or H502W and has one, two or three of the following substitutions S97M, L98E Y147R, F175V, F175L, F175I, G483S or T484W of SEQ ID NO: 2 or 13. In some embodiments, the variant having decreased thermostability has one or two of the following substitutions: F29V, I43Q, Y48V, F116M, H502S, or H502E and has one, two or three of the following substitutions S97M, L98E Y147R, F175V, F175L, F175I, G483S or T484W of SEQ ID NO: 2 or 13.

Variant Glucoamylases with Altered Specific Activity

As used herein, specific activity is the activity of the glucoamylase per mg of protein. Activity was determined using the glucoamylase assay using the chromogenic pNP-β-maltoside substrate. The screening identified variants having a Performance Index (PI) >1.0 or (PI)=1.0 compared to the parent TrGA PI. The PI is calculated from the specific activities (activity/mg enzyme) of the wild-type (WT) and the variant enzymes. It is the quotient “Variant-specific activity/WT-specific activity” and can be a measure of the increase in specific activity of the variant. A PI of about 2 should be about 2 fold better than WT. In some aspects, the disclosure relates to a variant glucoamylase having altered specific activity as compared to a parent or wild-type glucoamylase. In some embodiments, the altered specific activity is increased specific activity. Increased specific activity can be defined as an increased performance index of greater than 1, including greater than or equal to about 1.1, about 1.2, about 1.3, about 1.4, about 1.5, about 1.6, about 1.7, about 1.8, about 1.9, and about 2. In some embodiments, the increased specific activity is from about 1.0 to about 5.0, including about 1.1, about 1.2, about 1.3, about 1.4, about 1.5, about 1.6, about 1.7, about 1.8, about 1.9, about 2.0, about 2.1, about 2.2., about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, and about 4.9. In some embodiments, the variant has an at least about 1.0 fold higher specific activity than the parent glucoamylase, including at least about 1.1 fold, about 1.2 fold, about 1.3 fold, about 1.4 fold, about 1.5 fold, about 1.6 fold, about 1.7 fold, about 1.8 fold, about 1.9 fold, about 2.0 fold, about 2.2 fold, about 2.5 fold, about 2.7 fold, about 2.9 fold, about 3.0 fold, about 4.0 fold, and about 5.0 fold. In some embodiments, the specific activity is similar or equal to the parent. Thus, similar specific activity can be defined as an performance index that is 0.1 greater, equal or 0.1 less than to 1.0 of the parent, including about 0.02 greater or less than to 1.0, including about 0.04 greater or less than to 1.0, including about 0.06 greater or less than to 1.0, including about 0.08 greater or less than to 1.0 and including about 0.1 greater or less than to 1.0.

In some embodiments, variants having an improvement in specific activity include one or more deletions, substitutions or insertions, and particularly substitutions in the following positions in the amino acid sequence set forth in SEQ ID NO: 2: one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase. In some embodiments, variants having an improvement in specific activity include one or more deletions, substitutions or insertions, and particularly substitutions in the following positions in the amino acid sequence set forth in SEQ ID NO: 2 or 13: one or two amino acid substitutions in the group of interface amino acids consisting of residues 29, 43, 48, 116, and 502 of SEQ ID NO: 2 or 13; and one, two or three amino acid substitutions in the group of catalytic core amino acid residues consisting of residues 97, 98, 147, 175, 483 and 484 of SEQ ID NO: 2 or 13. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO: 2 or 13. In some embodiments, the parent glucoamylase will also have structural identity to SEQ ID NO: 2. In some embodiments, the variant having an improvement in specific activity has one or two of the following substitutions: F29V, F29Q, I43Q, Y48V, F116M, H502S, H502E, or H502W, and has one, two or three of the following substitutions S97M, L98E Y147R, F175V, F175L, F175I, G483S or T484W of SEQ ID NO: 2 or 13.

In some embodiments, the specific activity of the parent as compared to the variant is determined as described in the Assays and Methods.

Variant Glucoamylases with Altered Saccharification Performance

As used herein, the performance of the glucoamylase to facilitate starch saccharification in the fermenting vessel (FV) was determined indirect by the real degree of fermentation. The real degree of fermentation was determined in malt-adjunct brew experiments with the glucoamylase variants dosed either on GAU activity or on protein under a defined set of conditions. Real Degree of Fermentation (RDF, which is the Real Attenuation expressed in percentage form) was calculated for the final fermented wort (beer), as the specific gravity of the wort before, during and after fermentation was measured using a specific gravity hydrometer or Anton-Paar density meter (e.g. DMA 4100 M). Real Attenuation was calculated and expressed in percentage form as RDF according to the formulae listed by Ensminger (see http://hbd.org/ensmingr/ “Beer data: Alcohol, Calorie, and Attenuation Levels of Beer”).

In some aspects, the disclosure relates to a variant glucoamylase having altered RDF performance as compared to a parent or wild-type glucoamylase. In some embodiments, the altered RDF performance is similar or equal to the parent. TrGA has a RDF performance of 75.04% when dosed with 0.058 mg GA/ml wort. Thus, similar RDF performance can be defined as an obtained RDF value, under the described set of conditions and dosing 0.058 mg GA/ml wort, that is 0.5% greater, equal or 0.5% less than to 75.04%, including about 0.1% greater or less than to 75.04%, about 0.2% greater or less than to 75.04%, about 0.3% greater or less than to 75.04%, about 0.4% greater or less than to 75.04% or about 0.5% greater or less than to 75.04%.

In some embodiments, variants having similar real degree of fermentation as compared to the parent glucoamylase such as TrGA and include one or more deletions, substitutions or insertions, and particularly substitutions in the following positions in the amino acid sequence set forth in SEQ ID NO: 2: one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase. In some embodiments, variants of the disclosure having improved RDF performance include one or more deletions, substitutions or insertions, and particularly substitutions in the following positions in the amino acid sequence set forth in SEQ ID NO: 2 or 13: one or two amino acid substitutions in the group of interface amino acids consisting of residues 29, 43, 48, 116, and 502 of SEQ ID NO: 2 or 13; and one, two or three amino acid substitutions in the group of catalytic core amino acid residues consisting of residues 97, 98, 147, 175, 483 and 484 of SEQ ID NO: 2 or 13. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO: 2 or 13. In some embodiments, the parent glucoamylase will also have structural identity to SEQ ID NO: 2. In some embodiments, variants of the disclosure having improved RDF performance has one or two of the following substitutions: F29V, F29Q, I43Q, Y48V, F116M, H502S, H502E, or H502W and has one, two or three of the following substitutions S97M, L98E Y147R, F175V, F175L, F175I, G483S or T484W of SEQ ID NO: 2 or 13.

In some embodiments, the RDF performance of the parent as compared to the variant is determined as described in the Assays and Methods.

Variant Glucoamylases with Decreased Thermostability and Similar Saccharification Performance Compared to the Parent Glucoamylse

In some aspects, the disclosure relates to a variant glucoamylase having altered thermostability, and similar saccharification (RDF) performance as compared to a parent (e.g., wild-type). In some embodiments, the altered thermostability is a decreased thermostability, e.g. a more thermolabile variant. In some embodiments, the RDF performance is a similar RDF performance as compared to the parent glucoamylase.

In some embodiments, variants with a decreased thermostability and similar RDF performance include one or more deletions, substitutions or insertions, and particularly substitutions in the following positions in the amino acid sequence set forth in SEQ ID NO: 2: one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase. In some embodiments, variants with a decreased thermostability and similar RDF performance include one or more deletions, substitutions or insertions, and particularly substitutions in the following positions in the amino acid sequence set forth in SEQ ID NO: 2 or 13: one or two amino acid substitutions in the group of interface amino acids consisting of residues 29, 43, 48, 116, and 502 of SEQ ID NO: 2 or 13; and one, two or three amino acid substitutions in the group of catalytic core amino acid residues consisting of residues 97, 98, 147, 175, 483 and 484 of SEQ ID NO: 2 or 13. In some embodiments, the parent glucoamylase will be a Trichoderma glucoamylase homologue and in further embodiments, the parent glucoamylase will have at least about 50%, about 60%, about 70%, about 80%, about 90%, about 95%, or about 98% sequence identity to SEQ ID NO: 2 or 13. In some embodiments, the parent glucoamylase will also have structural identity to SEQ ID NO: 2. In some embodiments, the variant with a decreased thermostability and similar RDF performance has one or two of the following substitutions: F29V, F29Q, I43Q, Y48V, F116M, H502S, H502E, or H502W, and has one, two or three of the following substitutions S97M, L98E Y147R, F175V, F175L, F175I, G483S or T484W of SEQ ID NO: 2 or 13.

Variant Glucoamylases with Production of Fermentable Sugar(s)

In a further aspect, the glucoamylase exhibit an enhanced production of fermentable sugar(s) as compared to the parent glucoamylase such as TrGA. In a further aspect, the glucoamylase exhibit an enhanced production of fermentable sugars in the mashing step of the brewing process as compared to the parent glucoamylase such as TrGA. In a further aspect, the glucoamylase exhibit an enhanced production of fermentable sugars in the fermentation step of the brewing process as compared to the parent glucoamylase such as TrGA. In a further aspect, the fermentable sugar is glucose. A skilled person within the field can determine the production of fermentable sugar(s) by e.g. HPLC techniques.

### 4. POLYNUCLEOTIDES ENCODING GLUCOAMYLASES

The present disclosure also relates to isolated polynucleotides encoding the variant glucoamylase. The polynucleotides may be prepared by established techniques known in the art. The polynucleotides may be prepared synthetically, such as by an automatic DNA synthesizer. The DNA sequence may be of mixed genomic (or cDNA) and synthetic origin prepared by ligating fragments together. The polynucleotides may also be prepared by polymerase chain reaction (PCR) using specific primers. In general, reference is made to Minshull J. et al., Methods 32(4):416-427 (2004). DNA may also be synthesized by a number of commercial companies such as Geneart AG, Regensburg, Germany.

The present disclosure also provides isolated polynucleotides comprising a nucleotide sequence (i) having at least about 50% identity to SEQ ID NO: 4, including at least about 60%, about 70%, about 80%, about 90%, about 95%, and about 99%, or (ii) being capable of hybridizing to a probe derived from the nucleotide sequence set forth in SEQ ID NO: 4, under conditions of intermediate to high stringency, or (iii) being complementary to a nucleotide sequence having at least 90% sequence identity to the sequence set forth in SEQ ID NO: 4. Probes useful according to the disclosure may include at least about 50, about 100, about 150, about 200, about 250, about 300 or more contiguous nucleotides of SEQ ID NO: 4. In some embodiments, the encoded variant also has structural identity to SEQ ID NO: 2.

The present disclosure further provides isolated polynucleotides that encode variant glucoamylases that comprise an amino acid sequence comprising at least about 50%, about 60%, about 70%, about 80%, about 90%, about 93%, about 95%, about 97%, about 98%, or about 99% amino acid sequence identity to SEQ ID NO: 2 or SEQ ID NO: 13. Additionally, the present disclosure provides expression vectors comprising any of the polynucleotides provided above. The present disclosure also provides fragments (i.e., portions) of the DNA encoding the variant glucoamylases provided herein. These fragments find use in obtaining partial length DNA fragments capable of being used to isolate or identify polynucleotides encoding mature glucoamylase enzymes described herein from filamentous fungal cells (e.g., Trichoderma, Aspergillus, Fusarium, Penicillium, and Humicola), or a segment thereof having glucoamylase activity. In some embodiments, fragments of the DNA may comprise at least about 50, about 100, about 150, about 200, about 250, about 300 or more contiguous nucleotides. In some embodiments, portions of the DNA provided in SEQ ID NO: 4 may be used to obtain parent glucoamylases and particularly Trichoderma glucoamylase homologues from other species, such as filamentous fungi that encode a glucoamylase.

### 5. PRODUCTION OF GLUCOAMYLASES

### DNA Constructs and Vectors

According to one embodiment of the disclosure, a DNA construct comprising a polynucleotide as described above encoding a variant glucoamylase encompassed by the disclosure and operably linked to a promoter sequence is assembled to transfer into a host cell. In one aspect, a polynucleotide encoding a glucoamylase variant as disclosed herein is provided.

The DNA construct may be introduced into a host cell using a vector. In one aspect, a vector comprising the polynucleotide, or capable of expressing a glucoamylase variant as disclosed herein is provided. The vector may be any vector that when introduced into a host cell is stably introduced. In some embodiments, the vector is integrated into the host cell genome and is replicated. Vectors include cloning vectors, expression vectors, shuttle vectors, plasmids, phage particles, cassettes and the like. In some embodiments, the vector is an expression vector that comprises regulatory sequences operably linked to the glucoamylase coding sequence.

Examples of suitable expression and/or integration vectors are provided in Sambrook et al. (1989) supra, and Ausubel (1987) supra, and van den Hondel et al. (1991) in Bennett and Lasure (Eds.) More Gene Manipulations In Fungi, Academic Press pp. 396-428 and U.S. Pat. No. 5,874,276. Reference is also made to the Fungal Genetics Stock Center Catalogue of Strains (FGSC, http://www.fgsc.net) for a list of vectors. Particularly useful vectors include vectors obtained from for example Invitrogen and Promega.

Suitable plasmids for use in bacterial cells include pBR322 and pUC19 permitting replication in E. coli and pE194 for example permitting replication in Bacillus. Other specific vectors suitable for use in E. coli host cells include vectors such as pFB6, pBR322, pUC18, pUC100, pDONR™201, 10 pDONR™221, pENTR™, pGEM® 3Z and pGEM® 4Z.

Specific vectors suitable for use in fungal cells include pRAX, a general purpose expression vector useful in Aspergillus, pRAX with a giaA promoter, and in Hypocrea/Trichoderma includes pTrex3g with a cbh1 promoter.

In some embodiments, the promoter that shows transcriptional activity in a bacterial or a fungal host cell may be derived from genes encoding proteins either homologous or heterologous to the host cell. The promoter may be a mutant, a truncated and/or a hybrid promoter. The above-mentioned promoters are known in the art. Examples of suitable promoters useful in fungal cells and particularly filamentous fungal cells such as Trichoderma or Aspergillus cells include such exemplary promoters as the T. reesei promoters cbh1, cbh2, egi1, egi2, eg5, xln1 and xln2. Other examples of useful promoters include promoters from A. awamori and A. niger glucoamylase genes (giaA) (see Nunberg et al., Mol. Cell Biol. 4: 2306-2315 (1984) and Boel et al., EMBO J. 3:1581-1585 (1984)), A. oryzae TAKA amylase promoter, the TPI (triose phosphate isomerase) promoter from S. cerevisiae, the promoter from Aspergillus nidulans acetamidase genes and Rhizomucor miehei lipase genes. Examples of suitable promoters useful in bacterial cells include those obtained from the E. coli lac operon; Bacillus licheniformis alpha-amylase gene (amyL), B. stearothermophilus amylase gene (amyS); Bacillus subtilis xylA and xylB genes, the beta-lactamase gene, and the tac promoter. In some embodiments, the promoter is one that is native to the host cell. For example, when T. reesei is the host, the promoter is a native T. reesei promoter. In other embodiments, the promoter is one that is heterologous to the fungal host cell. In some embodiments, the promoter will be the promoter of a parent glucoamylase (e.g., the TrGA promoter).

In some embodiments, the DNA construct includes nucleic acids coding for a signal sequence, that is, an amino acid sequence linked to the amino terminus of the polypeptide that directs the encoded polypeptide into the cell's secretory pathway. The 5′ end of the coding sequence of the nucleic acid sequence may naturally include a signal peptide coding region that is naturally linked in translation reading frame with the segment of the glucoamylase coding sequence that encodes the secreted glucoamylase or the 5′ end of the coding sequence of the nucleic acid sequence may include a signal peptide that is foreign to the coding sequence. In some embodiments, the DNA construct includes a signal sequence that is naturally associated with a parent glucoamylase gene from which a variant glucoamylase has been obtained. In some embodiments, the signal sequence will be the sequence depicted in SEQ ID NO: 1 or a sequence having at least about 90%, about 94, or about 98% sequence identity thereto. Effective signal sequences may include the signal sequences obtained from other filamentous fungal enzymes, such as from Trichoderma (T. reesei glucoamylase, cellobiohydrolase I, cellobiohydrolase II, endoglucanase I, endoglucanase II, endoglucanase II, or a secreted proteinase, such as an aspartic proteinase), Humicola (H. insolens cellobiohydrolase or endoglucanase, or H. grisea glucoamylase), or Aspergillus (A. niger glucoamylase and A. oryzae TAKA amylase).

In additional embodiments, a DNA construct or vector comprising a signal sequence and a promoter sequence to be introduced into a host cell are derived from the same source. In some embodiments, the native glucoamylase signal sequence of a Trichoderma glucoamylase homologue, such as a signal sequence from a Hypocrea strain may be used.

In some embodiments, the expression vector also includes a termination sequence. Any termination sequence functional in the host cell may be used in the present disclosure. In some embodiments, the termination sequence and the promoter sequence are derived from the same source. In another embodiment, the termination sequence is homologous to the host cell. Useful termination sequences include termination sequences obtained from the genes of Trichoderma reesei cbl1; A. niger or A. awamori glucoamylase (Nunberg et al. (1984) supra, and Boel et al., (1984) supra), Aspergillus nidulans anthranilate synthase, Aspergillus oryzae TAKA amylase, or A. nidulans trpC (Punt et al., Gene 56:117-124 (1987)).

In some embodiments, an expression vector includes a selectable marker. Examples of selectable markers include ones that confer antimicrobial resistance (e.g., hygromycin and phleomycin). Nutritional selective markers also find use in the present disclosure including those markers known in the art as amdS (acetamidase), argB (ornithine carbamoyltransferase) and pyrG (orotidine-5′phosphate decarboxylase). Markers useful in vector systems for transformation of Trichoderma are known in the art (see, e.g., Finkelstein, Chapter 6 in Biotechnology Of Filamentous Fungi, Finkelstein et al. (1992) Eds. Butterworth-Heinemann, Boston, Mass.; Kinghorn et al. (1992) Applied Molecular Genetics Of Filamentous Fungi, Blackie Academic and Professional, Chapman and Hall, London; Berges and Barreau, Curr. Genet. 19:359-365 (1991); and van Hartingsveldt et al., Mol. Gen. Genet. 206:71-75 (1987)). In some embodiments, the selective marker is the amdS gene, which encodes the enzyme acetamidase, allowing transformed cells to grow on acetamide as a nitrogen source. The use of A. nidulans amdS gene as a selective marker is described in Kelley et al., EMBO J. 4:475-479 (1985) and Penttila et al., Gene 61:155-164 (1987).

Methods used to ligate the DNA construct comprising a nucleic acid sequence encoding a variant glucoamylase, a promoter, a termination and other sequences and to insert them into a suitable vector are well known in the art. Linking is generally accomplished by ligation at convenient restriction sites. If such sites do not exist, synthetic oligonucleotide linkers are used in accordance with conventional practice (see Sambrook et al. (1989) supra, and Bennett and Lasure, More Gene Manipulations In Fungi, Academic Press, San Diego (1991) pp 70-76.). Additionally, vectors can be constructed using known recombination techniques (e.g., Invitrogen Life Technologies, Gateway Technology).

### Host Cells and Transformation of Host Cells

The present disclosure also relates to host cells comprising a polynucleotide encoding a variant glucoamylase of the disclosure. In some embodiments, the host cells are chosen from bacterial, fungal, plant and yeast cells. The term host cell includes both the cells, progeny of the cells and protoplasts created from the cells that are used to produce a variant glucoamylase according to the disclosure. In one aspect, a host cell comprising, preferably transformed with a vector is disclosed. In a further aspect, a cell capable of expressing a glucoamylase variant is provided. In a further aspect, the host cell is a protease deficient and/or xylanase deficient and/or glucanase deficient host cell. A protease deficient and/or xylanase deficient and/or native glucanase deficient host cell may be obtained by deleting or silencing the genes coding for the mentioned enzymes. As a consequence the host cell containing the GA-variant is not expressing the mentioned enzymes

In some embodiments, the host cells are fungal cells and optionally filamentous fungal host cells. The term “filamentous fungi” refers to all filamentous forms of the subdivision Eumycotina (see, Alexopoulos, C. J. (1962), Introductory Mycology, Wiley, New York). These fungi are characterized by a vegetative mycelium with a cell wall composed of chitin, cellulose, and other complex polysaccharides. The filamentous fungi of the present disclosure are morphologically, physiologically, and genetically distinct from yeasts. Vegetative growth by filamentous fungi is by hyphal elongation and carbon catabolism is obligatory aerobic. In the present disclosure, the filamentous fungal parent cell may be a cell of a species of, but not limited to, Trichoderma (e.g., Trichoderma reesei, the asexual morph of Hypocrea jecorina, previously classified as T. longibrachiatum, Trichoderma viride, Trichoderma koningii, Trichoderma harzianum) (Sheir-Neirs et al., Appl. Microbiol. Biotechnol. 20:46-53 (1984); ATCC No. 56765 and ATCC No. 26921), Penicillium sp., Humicola sp. (e.g., H. insolens, H. lanuginosa and H. grisea), Chrysosporium sp. (e.g., C. lucknowense), Gliocladium sp., Aspergillus sp. (e.g., A. oryzae, A. niger, A sojae, A. japonicus, A. nidulans, and A. awamori) (Ward et al., Appl. Microbiol. Biotechnol. 39:738-743 (1993) and Goedegebuur et al., Curr. Genet. 41:89-98 (2002)), Fusarium sp., (e.g., F. roseum, F. graminum, F. cerealis, F. oxysporum, and F. venenatum), Neurospora sp., (N. crassa), Hypocrea sp., Mucorsp. (M. miehei), Rhizopus sp., and Emericella sp. (see also, Innis et al., Science 228:21-26 (1985)). The term “Trichoderma” or “Trichoderma sp.” or “Trichoderma spp.” refer to any fungal genus previously or currently classified as Trichoderma.

In some embodiments, the host cells will be gram-positive bacterial cells. Non-limiting examples include strains of Streptomyces (e.g., S. lividans, S. coelicolor, and S. griseus) and Bacillus. As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus tearothermophilus.”

In some embodiments, the host cell is a gram-negative bacterial strain, such as E. coli or Pseudomonas sp. In other embodiments, the host cells may be yeast cells such as Saccharomyces sp., Schizosaccharomyces sp., Pichia sp., or Candida sp. In other embodiments, the host cell will be a genetically engineered host cell wherein native genes have been inactivated, for example by deletion in bacterial or fungal cells. Where it is desired to obtain a fungal host cell having one or more inactivated genes known methods may be used (e.g., methods disclosed in U.S. Pat. No. 5,246,853, U.S. Pat. No. 5,475,101, and WO 92/06209). Gene inactivation may be accomplished by complete or partial deletion, by insertional inactivation or by any other means that renders a gene nonfunctional for its intended purpose (such that the gene is prevented from expression of a functional protein). In some embodiments, when the host cell is a Trichoderma cell and particularly a T. reesei host cell, the cbh1, cbh2, egl1 and egl2 genes will be inactivated and/or deleted. Exemplary Trichoderma reesei host cells having quad-deleted proteins are set forth and described in U.S. Pat. No. 5,847,276 and WO 05/001036. In other embodiments, the host cell is a protease deficient or protease minus strain.

Introduction of a DNA construct or vector into a host cell includes techniques such as transformation; electroporation; nuclear microinjection; transduction; transfection, (e.g., lipofection-mediated and DEAE-Dextrin mediated transfection); incubation with calcium phosphate DNA precipitate; high velocity bombardment with DNA-coated microprojectiles; and protoplast fusion. General transformation techniques are known in the art (see, e.g., Ausubel et al. (1987) supra, chapter 9; and Sambrook et al. (1989) supra, and Campbell et al., Curr. Genet. 16:53-56 (1989)).

Transformation methods for Bacillus are disclosed in numerous references including Anagnostopoulos C. and J. Spizizen, J. Bacteriol. 81:741-746 (1961) and WO 02/14490.

Transformation methods for Aspergillus are described in Yelton et al., Proc. Natl. Acad. Sci. USA 81:1470-1474 (1984); Berka et al., (1991) in Applications of Enzyme Biotechnology, Eds. Kelly and Baldwin, Plenum Press (NY); Cao et al., Protein Sci. 9:991-1001 (2000); Campbell et al., Curr. Genet. 16:53-56 (1989), and EP 238 023. The expression of heterologous protein in Trichoderma is described in U.S. Pat. No. 6,022,725; U.S. Pat. No. 6,268,328; Harkki et al. Enzyme Microb. Technol. 13:227-233 (1991); Harkki et al., BioTechnol. 7:596-603 (1989); EP 244,234; EP 215,594; and Nevalainen et al., “The Molecular Biology of Trichoderma and its Application to the Expression of Both Homologous and Heterologous Genes”, in Molecular Industrial Mycology, Eds. Leong and Berka, Marcel Dekker Inc., NY (1992) pp. 129-148). Reference is also made to WO96/00787 and Bajar et al., Proc. Natl. Acad. Sci. USA 88:8202-8212 (1991) for transformation of Fusarium strains.

In one specific embodiment, the preparation of Trichoderma sp. for transformation involves the preparation of protoplasts from fungal mycelia (see, Campbell et al., Curr. Genet. 16:53-56 (1989); Pentilla et al., Gene 61:155-164 (1987)). Agrobacterium tumefaciens-mediated transformation of filamentous fungi is known (see de Groot et al., Nat. Biotechnol. 16:839-842 (1998)). Reference is also made to U.S. Pat. No. 6,022,725 and U.S. Pat. No. 6,268,328 for transformation procedures used with filamentous fungal hosts.

In some embodiments, genetically stable transformants are constructed with vector systems whereby the nucleic acid encoding the variant glucoamylase is stably integrated into a host strain chromosome. Transformants are then purified by known techniques.

In some further embodiments, the host cells are plant cells, such as cells from a monocot plant (e.g., corn (maize), wheat, and sorghum) or cells from a dicot plant (e.g., soybean). Methods for making DNA constructs useful in transformation of plants and methods for plant transformation are known. Some of these methods include Agrobacterium tumefaciens mediated gene transfer; microprojectile bombardment, PEG mediated transformation of protoplasts, electroporation and the like. Reference is made to U.S. Pat. No. 6,803,499, U.S. Pat. No. 6,777,589; Fromm et al., BioTechnol. 8:833-839 (1990); Potrykus et al., Mol. Gen. Genet. 199:169-177 (1985).

### Production of Glucoamylases

The present disclosure further relates to methods of producing the variant glucoamylases, which comprises transforming a host cell with an expression vector comprising a polynucleotide encoding a variant glucoamylase according to the disclosure, culturing the host cell under conditions suitable for expression and production of the variant glucoamylase and optionally recovering the variant glucoamylase. In one aspect, a method of expressing a variant glucoamylase according to the disclosure, the method comprising obtaining a host cell or a cell as disclosed herein and expressing the glucoamylase variant from the cell or host cell, and optionally purifying the glucoamylase variant, is provided. In one aspect, the glucoamylase variant is purified.

In the expression and production methods of the present disclosure the host cells are cultured under suitable conditions in shake flask cultivation, small scale or large scale fermentations (including continuous, batch and fed batch fermentations) in laboratory or industrial fermentors, with suitable medium containing physiological salts and nutrients (see, e.g., Pourquie, J. et al., Biochemistry And Genetics Of Cellulose Degradation, eds. Aubert, J. P. et al., Academic Press, pp. 71-86, 1988 and Ilmen, M. et al., Appl. Environ. Microbiol. 63:1298-1306 (1997)). Common commercially prepared media (e.g., Yeast Malt Extract (YM) broth, Luria Bertani (LB) broth and Sabouraud Dextrose (SD) broth) find use in the present disclosure. Culture conditions for bacterial and filamentous fungal cells are known in the art and may be found in the scientific literature and/or from the source of the fungi such as the American Type Culture Collection and Fungal Genetics Stock Center. In cases where a glucoamylase coding sequence is under the control of an inducible promoter, the inducing agent (e.g., a sugar, metal salt or antimicrobial), is added to the medium at a concentration effective to induce glucoamylase expression.

In some embodiments, the present disclosure relates to methods of producing the variant glucoamylase in a plant host comprising transforming a plant cell with a vector comprising a polynucleotide encoding a glucoamylase variant according to the disclosure and growing the plant cell under conditions suitable for the expression and production of the variant.

In some embodiments, assays are carried out to evaluate the expression of a variant glucoamylase by a cell line that has been transformed with a polynucleotide encoding a variant glucoamylase encompassed by the disclosure. The assays can be carried out at the protein level, the RNA level and/or by use of functional bioassays particular to glucoamylase activity and/or production. Some of these assays include Northern blotting, dot blotting (DNA or RNA analysis), RT-PCR (reverse transcriptase polymerase chain reaction), in situ hybridization using an appropriately labeled probe (based on the nucleic acid coding sequence) and conventional Southern blotting and autoradiography.

In addition, the production and/or expression of a variant glucoamylase may be measured in a sample directly, for example, by assays directly measuring reducing sugars such as glucose in the culture medium and by assays for measuring glucoamylase activity, expression and/or production. In particular, glucoamylase activity may be assayed by the 3,5-dinitrosalicylic acid (DNS) method (see Goto et al., Biosci. Biotechnol. Biochem. 58:49-54 (1994)). In additional embodiments, protein expression, is evaluated by immunological methods, such as immunohistochemical staining of cells, tissue sections or immunoassay of tissue culture medium, (e.g., by Western blot or ELISA). Such immunoassays can be used to qualitatively and quantitatively evaluate expression of a glucoamylase. The details of such methods are known to those of skill in the art and many reagents for practicing such methods are commercially available.

The glucoamylases of the present disclosure may be recovered or purified from culture media by a variety of procedures known in the art including centrifugation, filtration, extraction, precipitation and the like.

In some embodiments, a glucoamylase variant will have more than one amino acid substitution. For example, the variant may have 1, 2, 3, 4, 5, 6, 7, 8, 9, 10, 15, 20, or 25 amino acid substitutions, deletions, or insertions as compared to a parent glucoamylase. In some embodiments, a glucoamylase variant comprises a substitution, deletion, or insertion in at least one amino acid position in a position corresponding to the regions of non-conserved amino acids. As contemplated herein, the glucoamylase variants can have substitutions, deletions, or insertions in any position in the mature protein sequence.

As contemplated herein, a DNA sequence encoding glucoamylase or a glucoamylase variant can be expressed, in enzyme form, using an expression vector which typically includes control sequences encoding a promoter, operator, ribosome binding site, translation initiation signal, and, optionally, a repressor gene or various activator genes. The recombinant expression vector carrying the DNA sequence encoding a glucoamylase as contemplated herein may be any vector which may conveniently be subjected to recombinant DNA procedures. The vector may be one which, when introduced into a parent glucoamylase, is integrated into the genome and replicated together with the chromosome(s) into which it has been integrated. For example, the fungal cell may be transformed with the DNA construct encoding the glucoamylase, and integrating the DNA construct, in one or more copies, in the host chromosome(s). This integration is generally considered to be an advantage, as the DNA sequence is more likely to be stably maintained. Integration of the DNA constructs into the host chromosome may be performed according to conventional methods, such as by homologous or heterologous recombination.

In an embodiment incorporating use of a vector, the DNA sequence should be operably connected to a suitable promoter sequence. The promoter may be any DNA sequence which shows transcriptional activity in a parent glucoamylase and may be derived from genes encoding proteins either homologous or heterologous to A parent glucoamylase. Examples of suitable promoters for directing the transcription of the DNA sequence encoding a glucoamylase variant are, by non-limiting example only, those derived from the gene encoding A. oryzae TAKA amylase, the T. reesei cellobiohydrolase I, Rhizomucor miehei aspartic proteinase, A. niger neutral α-amylase, A. niger acid stable α-amylase, A. niger glucoamylase, Rhizomucor miehei lipase, A. oryzae alkaline protease, or A. nidulans glyceraldehyde-3-phosphate dehydrogenase A. Any expression vector as contemplated may also comprise a suitable transcription terminator and polyadenylation sequences operably connected to the DNA sequence encoding the glucoamylase or variant. Termination and polyadenylation sequences may suitably be derived from the same sources as the promoter. The vector may further comprise any DNA sequence enabling or effectuating the vector to replicate in the fungal host. The vector may also comprise additional genes, the product of which may complement a defect in the fungal host. For example, selectable markers may be incorporated to provide drug resistance. As contemplated herein, all procedures used to ligate DNA constructs encoding a glucoamylase, the promoter, terminator and other elements, respectively, and to insert them into suitable vectors containing the information necessary for replication, are those as may be understood by persons skilled in the art.

In one aspect the invention relates to a host cell having heterologous expression of a polypeptide as described herein such as a fungal cell for example of the genus Trichoderma such as Trichoderma reesei. In another aspect, the fungal cell is of the species Hypocrea jecorina.

In one aspect, the host cell comprises, or is preferably transformed with, a plasmid or an expression vector and is therefore capable of expressing a polypeptide as contemplated herein. In one aspect, the expression vector comprises a nucleic acid and the expression vector or plasmid as contemplated herein may comprise a promoter derived from Trichoderma such as a T. reesei cbhI-derived promoter and/or the a terminator derived from Trichoderma such as a T. reesei cbhI-derived terminator and/or one or more selective markers such as Aspergillus nidulans amdS and pyrG and/or one or more telomere regions allowing for a non-chromosomal plasmid maintenance in a host cell.

### 6. COMPOSITIONS AND USES

The glucoamylases as contemplated herein may be used in compositions including but not limited to starch hydrolyzing and saccharifying compositions, cleaning and detergent compositions (e.g., laundry detergents, dish washing detergents, and hard surface cleaning compositions), alcohol fermentation compositions, and in animal feed compositions, for example. Further, these glucoamylases may be used in baking applications, such as bread and cake production, brewing, healthcare, textile, environmental waste conversion processes, biopulp processing, and biomass conversion applications.

In some embodiments, a composition comprising a glucoamylase as contemplated herein will be optionally used in combination with any one or in any combination with the following enzymes—alpha amylases, beta-amylases, peptidases (proteases, proteinases, endopeptidases, exopeptidases), pullulanases, isoamylases, cellulases, hemicellulases, endo-glucanases and related beta-glucan hydrolytic accessory enzymes, xylanases and xylanase accessory enzymes, acetolactate decarboxylases, cyclodextrin glycotransferases, lipases, phytases, laccases, oxidases, esterases, cutinases, granular starch hydrolyzing enzymes and other glucoamylases.

In some embodiments, the composition will include the one or more further enzyme(s). In some embodiments, the composition will include the one or more further enzyme(s) selected among alpha-amylase, beta-amylase, peptidase (such as protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

In another embodiment, the variant(s) contemplated herein and/or one or more further enzyme(s) is inactivated by pasteurisation, such as by using less than 50, 45, 40, 35, 30, 25, 20, 19, 18, 17, 16 or 15 pasteurisation units (PU) in beer, such as Pilsner beer.

In some embodiments, the composition will include an alpha amylase such as fungal alpha amylases (e.g., Aspergillus sp.) or bacterial alpha amylases (e.g., Bacillus sp. such as B. stearothermophilus, (Geobacillus stearothermophilus), B. amyloliquefaciens, and B. licheniformis) and variants and hybrids thereof. In some embodiments, the alpha amylase is an acid stable alpha amylase. In some embodiments, the alpha amylase is Aspergillus kawachi alpha amylase (AkAA), see U.S. Pat. No. 7,037,704. Commercially available alpha amylases contemplated for use in the compositions of the disclosure are known and include GZYME® G-997, SPEZYME® FRED, SPEZYME® XTRA (Danisco US, Inc, Genencor Division), TERMAMYL 120-L and SUPRA (Novozymes, Biotech.).

In some embodiments, the composition will include an acid fungal protease. In a further embodiment, the composition will include the endo-protease (EC 3.4.21.26) sourced from a variant of the microorganism Aspergillus niger that hydrolyses peptides at the carboxyl site of proline residues disclosed in WO 2007/101888 published 13 Sep. 2007. In a further embodiment, the acid fungal protease is derived from a Trichoderma sp and may be any one of the proteases disclosed in US 2006/0154353, published Jul. 13, 2006, incorporated herein by reference. In a further embodiment, the composition will include a phytase from Buttiauxiella spp. (e.g., BP-17, see also variants disclosed in PCT patent publication WO 2006/043178). In a further embodiment, the composition will include an acetolactate decarboxylase (ALDC) EC 4.1.1.5, for example from Bacillus licheniformis or from the ALDC gene of Bacillus brevis expressed in a modified strain of Bacillus subtilis as disclosed in U.S. Pat. No. 4,617,273 published Oct. 14, 1986.

In other embodiments, the glucoamylases as contemplated herein may be combined with other such glucoamylases. In some embodiments, such glucoamylases will be combined with one or more glucoamylases derived from other various strains or variants of Monascus kaoliang, or of Aspergillus or variants thereof, such as A. oryzae, A. niger, A. kawachi, and A. awamori; glucoamylases derived from strains of Humicola or variants thereof; glucoamylases derived from strains of Talaromyces or variants thereof, such as T. emersonii; glucoamylases derived from strains of Athelia, such as A. rolfsii; or glucoamylases derived from strains of Penicillium, such as P. chrysogenum, for example.

In particular, glucoamylases as contemplated herein may be used for starch conversion processes, and particularly in the production of dextrose for fructose syrups, specialty sugars and in alcohol and other end-product (e.g., organic acid, ascorbic acid, and amino acids) production from fermentation of starch containing substrates (G. M. A. van Beynum et al., Eds. (1985) STARCH CONVERSION TECHNOLOGY, Marcel Dekker Inc. NY). Dextrins produced using variant glucoamylase compositions of the disclosure may result in glucose yields of at least 80%, at least 85%, at least 90% and at least 95%. Production of alcohol from the fermentation of starch substrates using glucoamylases as contemplated herein may include the production of fuel alcohol or potable alcohol. In some embodiments, the production of alcohol will be greater when variant glucoamylases are used under the same conditions as parent or wild-type glucoamylase. In some embodiments, the production of alcohol will be between about 0.5% and 2.5% better, including but not limited to 0.6%, 0.7%, 0.8%, 0.9%, 1.0%, 1.1%, 1.2%, 1.3%, 1.4%, 1.5%, 1.6%. 1.7%, 1.8%, 1.9%, 2.0%, 2.1%, 2.2%, 2.3%, and 2.4% more alcohol than the parent or wild-type glucoamylase.

In some embodiments, the glucoamylases as contemplated herein will find use in the hydrolysis of starch from various plant-based substrates, usually starch and/or sugar containing plant material, which are used for alcohol production. In some embodiments, the plant-based substrates will include corn (maize), wheat, barley, rye, milo, rice, sugar cane, potatoes, cassava and combinations thereof. In some embodiments, the plant-based substrate will be fractionated plant material, for example a cereal grain such as corn (maize), which is fractionated into components such as fiber, germ, protein and starch (endosperm) (U.S. Pat. No. 6,254,914 and U.S. Pat. No. 6,899,910). Methods of alcohol fermentations are described in THE ALCOHOL TEXTBOOK, A REFERENCE FOR THE BEVERAGE, FUEL AND INDUSTRIAL ALCOHOL INDUSTRIES, 3rd Ed., Eds K. A. Jacques et al., 1999, Nottingham University Press, UK. In certain embodiments, the alcohol will be ethanol. In particular, alcohol fermentation production processes are characterized as wet milling or dry milling processes. In some embodiments, the glucoamylase will be used in a wet milling fermentation process and in other embodiments the glucoamylase will find use in a dry milling process.

Dry grain milling involves a number of basic steps, which generally include: grinding, cooking, liquefaction, saccharification, fermentation, and separation of liquid and solids to produce alcohol and other co-products. Plant material and particularly whole cereal grains, such as corn (maize), wheat, or rye are ground. In some cases the grain may be first fractionated into component parts. The ground plant material may be milled to obtain a coarse or fine particle. The ground plant material can be mixed with liquid (e.g., water and/or thin stillage) in a slurry tank. The slurry is subjected to high temperatures (e.g., 90° C. to 105° C. or higher) in a jet cooker along with liquefying enzymes (e.g., alpha amylases) to solubilize and hydrolyze the starch in the grain to dextrins. The mixture can be cooled down and further treated with saccharifying enzymes, such as glucoamylases encompassed by the instant disclosure, to produce glucose. The mash containing glucose may then be fermented for approximately 24 to 120 hours in the presence of fermentation microorganisms, such as ethanol producing microorganism and particularly yeast (Saccharomyces spp). The solids in the mash are separated from the liquid phase and alcohol such as ethanol and useful co-products such as distillers' grains are obtained.

In some embodiments, the saccharification step and fermentation step are combined and the process is referred to as simultaneous saccharification and fermentation or simultaneous saccharification, yeast propagation and fermentation. In one aspect, the glucoamylase variants disclosed herein is used in a one-step process converting cellulosic biomass into alcohol that combines cellulytic enzymes and microbes for fermentation. In the process, the sugars released by enzymatic action may simultaneously be converted into alcohol by microbic fermentation.

In other embodiments, these glucoamylases may be used in a process for starch hydrolysis wherein the temperature of the process is between 25° C. and 50° C., in some embodiments, between 30° C. and 40° C. In some embodiments, the glucoamylase can be used in a process for starch hydrolysis at a pH of between pH 3.0 and pH 6.5. The fermentation processes in some embodiments include milling of a cereal grain or fractionated grain and combining the ground cereal grain with liquid to form a slurry that can then be mixed in a single vessel with a glucoamylase according to the disclosure and optionally other enzymes such as, but not limited to, alpha amylases, other glucoamylases, phytases, proteases, pullulanases, isoamylases or other enzymes having granular starch hydrolyzing activity and yeast to produce ethanol and other co-products (see e.g., U.S. Pat. No. 4,514,496, WO 04/081193, and WO 04/080923).

In some embodiments, the disclosure pertains to a method of saccharifying a liquid starch solution, which comprises an enzymatic saccharification step using one or more glucoamylases as contemplated herein.

In some embodiments, the disclosure pertains to a method of hydrolyzing and saccharifying gelatinised and liquefied (typically) grist starch to be used in brewing, whereby a composition comprising one or more glucoamylases as contemplated herein, is used to enhance the amount of brewers' yeast fermentable sugars obtained from the starch. A brewing process is used to produce the potable product, beer, where fermentable sugars are converted to ethanol and CO₂ by fermentation with brewers' yeast. The fermentable sugars are traditionally derived from starch in cereal grains, optionally supplemented with fermentable sugar sources such as glucose and maltose syrups and cane sugar. Briefly, beer production, well-known in the art, typically includes the steps of malting, mashing, and fermentation.

Historically the first step in beer production is malting—steeping, germination and drying of cereal grain (e.g. barley). During malting enzymes are produced in the germinating cereal (e.g. barley) kernel and there are certain changes in its chemical constituents (known as modification) including some degradation of starch, proteins and beta-glucans.

The malted cereal is milled to give a grist which may be mixed with a milled adjunct (e.g. non-germinated cereal grain) to give a mixed grist. The grist can also consist predominantly, or uniquely of adjunct. The grist is mixed with water and subjected to mashing; a previously cooked (gelatinised and liquefied) adjunct (the result of “adjunct cooking”) may be added to the mash. The mashing process is conducted over a period of time at various temperatures in order to hydrolyse cereal proteins, degrade beta-glucans and solubilise and hydrolyse the starch. The hydrolysis of the grist starch in the malt and adjunct in traditional mashing is believed to be catalysed by two main enzymes endogenous to malted barley. Alpha-amylase, randomly cleaves alpha-1,4 bonds in the interior of the starch molecule fragmenting them into smaller dextrins. Beta-amylase sequentially cleaves alpha-1,4 bonds from the non-reducing end of the these dextrins producing mainly maltose. Both alpha- and beta-amylase are unable to hydrolyse the alpha-1,6 bonds which forms the branching points of the starch chains in the starch molecule, which results in the accumulation of limit dextrins in the mash. Malt does contain an enzyme, limit dextrinase, which catalyses the hydrolysis of alpha-1,6 bonds but it only shows weak activity at mashing temperatures due to its thermolability. After mashing, the liquid extract (wort) is separated from the spent grain solids (i.e. the insoluble grain and husk material forming part of grist). The objectives of wort separation include: •to obtain good extract recovery, •to obtain good filterability, and •to produce clear wort. Extract recovery and filterability of the wort are important in the economics of the brewing process.

The composition of the wort depends on the raw materials, mashing process and profiles and other variables. A typical wort comprises 65-80% fermentable sugars (glucose, maltose and maltotriose, and 20-35% non-fermentable limit dextrins (sugars with a higher degree of polymerization than maltotriose). An insufficiency of starch hydrolytic enzymes during mashing can arise when brewing with high levels of adjunct unmalted cereal grists. A source of exogenous enzymes, capable of producing fermentable sugars during the mashing process is thus needed. Furthermore, such exogenous enzymes are also needed to reduce the level of non-fermentable sugars in the wort, with a corresponding increase in fermentable sugars, in order to brew highly attenuated beers with a low carbohydrate content. Herein disclosed is a enzyme composition for hydrolysis of starch comprising at least one glucoamylase as contemplated herein, which can be added to the mash or used in the mashing step of a brewing process, in order to cleave alpha-1,4 bonds and/or alpha-1,6 bonds in starch grist and thereby increase the fermentable sugar content of the wort and reduce the residue of non-fermentable sugars in the finished beer. In addition, the wort, so produced may be dried (by for example spray drying) or concentrated (e.g. boiling and evaporation) to provide a syrup or powder.

The grist, as contemplated herein, may comprise any starch and/or sugar containing plant material derivable from any plant and plant part, including e.g. tubers, roots, stems, leaves and seeds as described previously. Preferably the grist comprises grain, such as grain from barley, wheat, rye, oat, corn (maize), rice, milo, millet and sorghum, and more preferably, at least 10%, or more preferably at least 15%, even more preferably at least 25%, or most preferably at least 35%, such as at least 50%, at least 75%, at least 90% or even 100% (w/w) of the grist of the wort is derived from grain. Most preferably the grist comprises malted grain, such as barley malt. Preferably, at least 10%, or more preferably at least 15%, even more preferably at least 25%, or most preferably at least 35%, such as at least 50%, at least 75%, at least 90% or even 100% (w/w) of the grist of the wort is derived from malted grain. Preferably the grist comprises adjunct, such as non-malted grain from barley, wheat, rye, oat, corn (maize), rice, milo, millet and sorghum, and more preferably, at least 10%, or more preferably at least 15%, even more preferably at least 25%, or most preferably at least 35%, such as at least 50%, at least 75%, at least 90% or even 100% (w/w) of the grist of the wort is derived from non-malted grain or other adjunct. Adjunct comprising readily fermentable carbohydrates such as sugars or syrups may be added to the malt mash before, during or after the mashing process of the invention but is preferably added after the mashing process. A part of the adjunct may be treated with an alpha-amylase, and/or endopeptidase (protease) and/or a endoglucanase, and/or heat treated before being added to the mash. The enzyme composition for hydrolysis of starch, as contemplated herein, may include additional enzyme(s), preferably an enzyme selected from among an alpha-amylase, beta-amylase, peptidase (protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. During the mashing process, starch extracted from the grist is gradually hydrolyzed into fermentable sugars and smaller dextrins. Preferably the mash is starch negative to iodine testing, before wort separation.

After mashing, the wort (liquid extract wort) is separated from the spent grain solids by the process of lautering or mash filtration. The objectives of wort separation include: good extract recovery; good filterability, and a clear wort (further information may be found in “Technology Brewing and Malting” by Wolfgang Kunze of the Research and Teaching Institute of Brewing, Berlin (VLB), 3rd completely updated edition, 2004, ISBN 3-921690-49-8).

Prior to the third step of the brewing process, fermentation, the wort is typically transferred to a brew kettle and boiled vigorously for 50-60 minutes. A number of important processes occur during wort boiling (further information may be found in “Technology Brewing and Malting” by Wolfgang Kunze of the Research and Teaching Institute of Brewing, Berlin (VLB), 3rd completely updated edition, 2004, ISBN 3-921690-49-8) including inactivation of the endogenous malt enzymes and any exogenous enzyme added to the mash or adjunct. The boiled wort is then cooled, pitched with brewers' yeast and fermented at temperatures ranged from 8-16° C. to convert the fermentable sugars to ethanol. A low-alcohol beer can be produced from the final beer, by a process of vacuum evaporation that serves to selectively remove alcohol. Furthermore, hops may be added to the wort.

In one aspect, the invention relates to the use of a variant or a composition as contemplated herein in a fermentation, wherein said variant or composition is added before or during a fermentation step. In a further aspect, said fermentation step is followed by a pasteurisation step. In one aspect, said fermented beverage is selected from the group consisting of beer such as low alcohol beer or low calorie beer. In another aspect, said variant or said composition is added in combination with one or more further enzyme(s), such as selected among alpha-amylase, protease, pullulanase, isoamylase, cellulase, endoglucanase, xylanase, arabinofuranosidase, ferulic acid esterase, xylan acetyl esterase and glucoamylase, including any combination(s) thereof. In yet a further aspect, the variant and/or the one or more further enzyme(s) is inactivated in the pasteurisation step.

In one aspect, the variant(s) contemplated herein is added in an amount of for example 0.01-50 mg pr. ml fermented wort, such as 0.05-25 mg pr. ml fermented wort, such as 0.1-15 mg pr. ml fermented wort, such as 0.2-10 mg pr. ml fermented wort, such as 1-5 mg pr. ml fermented wort.

In one aspect, the variant(s) contemplated herein is added in an amount of for example at least 0.001, 0.01, 0.05, 0.10, 0.200, 0.300, 0.500, 0.800, 0.100, 0.500 or 1.000 mg pr. ml fermented wort.

In one aspect, the variant(s) contemplated herein is added in an amount of for example 0.01-20 GAU pr. ml fermented wort, such as 0.02-10 GAU pr. ml fermented wort, such as 0.05-5 GAU pr. ml fermented wort, such as 0.08-2 GAU pr. ml fermented wort, such as 0.1-1 GAU pr. ml fermented wort.

In one aspect, the variant(s) contemplated herein is added in an amount of for example at least 0.010, 0.050, 0.100, 0.150, 0.300, 0.500, 0.800, 1.00, 5.00 or 10.0 GAU pr. ml fermented wort.

In an alternative embodiment, the invention relates to a method, such as in a method wherein a fermentation is comprised in a process for making a fermented beverage, which method comprises adding a variant or a composition as described herein before or during a fermentation step, such as in a method comprising a pasteurisation step after the fermentation step or optional beer filtration step.

In one aspect, the invention relates to a method for production of a fermented beverage which comprises the following steps:

a) preparing a mash, such as obtained from a grist, where said grist for example comprises one or more of malted and/or unmalted grain, or starch-based material from another crop, and wherein the this step optionally further comprises contacting said mash with one or more further enzyme(s),
b) filtering the mash to obtain a wort, and
c) fermenting the wort to obtain a fermented beverage,
and optionally a pasteurisation step (d)
wherein a variant or a composition as described herein is added to:
i. the mash of step (a) and/or
ii. the wort of step (b) and/or
iii. the wort of step (c).

In one aspect the one or more enzymes optionally added in step a may be selected among a starch debranching enzyme, R-enzyme, limit dextrinase, alpha-amylase, beta-amylase, peptidase (protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof. In another aspect, one or more enzymes may also be added by contacting the wort of step (b) or (c) with one or more further enzyme(s), wherein the enzyme is selected among a starch debranching enzyme, isoamylase and limit dextrinase, including any combinations thereof.

In an alternative embodiment, the disclosure pertains to a method of enhancing the amount of fermentable sugars in the wort, using a composition comprising one or more glucoamylases as contemplated herein (e.g. thermolabile glucoamylase), whereby the composition is added to the wort after it has been boiled, such that the one or more glucoamylases are active during the fermentation step. The composition can be added to the boiled wort either before, simultaneously, or after the wort is pitched with the brewers' yeast. At the end of the fermentation and maturation step the beer, which may optionally be subjected to vacuum evaporation to produce a low-alcohol beer, is then optionally filtered and/or pasteurised. An inherent advantage of this method lies in the duration of the fermentation process, which is about 6-15 days (depending on pitching rate, fermentation, temperature, etc), which allows more time for the enzymatic cleavage of non-fermentable sugars, as compared to the short mashing step (2-4 h duration). A further advantage of this method lies in the amount of the composition needed to achieve the desired decrease in non-fermentable sugars (and increase in fermentable sugars), which corresponds to a significantly lower number of units of enzymatic activity (e.g. units of glucoamylase activity) than would need to be added to the mash to achieve a similar decrease in non-fermentable sugars. In addition, it removes the difficulties often seen during wort separation, especially by lautering, when high dose rates of glucoamylase are added in the mash. In contrast to alternative sources of glucoamylase enzyme, it has surprisingly been found that the glucoamylases as contemplated herein, are sufficiently temperature sensitive, that the final heat-treatment step of the finished beer (standard pasteurisation conditions) is sufficient for its catalytic activity to be inactivated. Hence an important advantage of the composition comprising one or more glucoamylases as contemplated herein, is that it can be used to reduce the amount of non-fermentable sugars in the wort during the fermentation step of brewing in order to brew highly attenuated beers with a low carbohydrate content, and where the catalytic activity of the composition is susceptible to inactivation by the heat treatment during beer pasteurisation thereby avoiding the expense of immobilized enzyme reactors or the use of genetically engineered brewer's yeast.

The present disclosure also provides a method for the production of a food, feed, or beverage product, such as an alcoholic or non-alcoholic beverage, such as a cereal- or malt-based beverage like beer or whiskey, such as wine, cider, vinegar, rice wine, soya sauce, or juice, said method comprising the step of treating a starch and/or sugar containing plant material with a variant or a composition as described herein. In another aspect, the invention also relates to a kit comprising a variant, or a composition as contemplated herein; and instructions for use of said variant or composition. The invention also relates to a fermented beverage produced by a method as described herein.

The present disclosure also provides an animal feed composition or formulation comprising at least one glucoamylase as contemplated herein. Methods of using a glucoamylase enzyme in the production of feeds comprising starch are provided in for example WO 03/049550 (herein incorporated by reference in its entirety). Briefly, the glucoamylase is admixed with a feed comprising starch. The glucoamylase is capable of degrading resistant starch for use by the animal.

Other objects and advantages of the present disclosure are apparent from the present specification.

### 6. FURTHER NUMBERED EMBODIMENTS ACCORDING TO THE INVENTION

### Embodiment 1

A glucoamylase variant comprising one or two amino acid substitutions in the group of interface amino acids consisting of residues 29, 43, 48, 116, and 502 of SEQ ID NO: 2, or an equivalent position in a parent glucoamylase; and one, two or three amino acid substitutions in the group of catalytic core amino acid residues consisting of residues 97, 98, 147, 175, 483 and 484 of SEQ ID NO: 2, or an equivalent position in a parent glucoamylase.

### Embodiment 2

A glucoamylase variant comprising

a) an amino acid substitution at the residue corresponding to position 502 of SEQ ID NO: 2, or an equivalent position in a parent glucoamylase, and optionally an amino acid substitution selected from the group of interface amino acids consisting of residues 29, 43, 48, and 116 of SEQ ID NO: 2, or an equivalent position in a parent glucoamylase;
b) an amino acid substitution at the residue corresponding to position 98 of SEQ ID NO: 2, or an equivalent position in a parent glucoamylase, and optionally one or two amino acid substitutions selected from the group of catalytic core amino acid residues consisting of residues 97, 147, 175, 483 and 484 of SEQ ID NO: 2, or an equivalent position in a parent glucoamylase;
which glucoamylase variant at least has one amino acid substitution selected from said group of interface amino acids or said group of catalytic core amino acid residues;
wherein the glucoamylase variant has at least 80% sequence identity with SEQ ID NO: 1, 2, 13, 18, 19, 20, 21, or 22.

### Embodiment 3

A glucoamylase variant comprising

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase, or an amino acid substitution at the residue corresponding to position 147 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

### Embodiment 4

A glucoamylase variant comprising

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 147 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

### Embodiment 5

A glucoamylase variant comprising

a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

### Embodiment 6

A glucoamylase variant comprising the amino acid substitution H502S of SEQ ID NO: 2 or 13; the amino acid substitution L98E of SEQ ID NO: 2 or 13; and the amino acid substitution Y48V of SEQ ID NO: 2 or 13, or the amino acid substitution Y147R of SEQ ID NO: 2 or 13; wherein the glucoamylase variant has at least 80% sequence identity with SEQ ID NO: 2 or 13.

### Embodiment 7

The glucoamylase variant according to any one of embodiments 1-5, wherein the parent glucoamylase is SEQ ID NO: 1, 2, 13, 18, 19, 20, 21, or 22.

### Embodiment 8

The glucoamylase variant according to any one of embodiments 1-7, wherein the parent glucoamylase is SEQ ID NO: 2 or 13.

### Embodiment 9

The glucoamylase variant according to any one of embodiments 1-8 comprising one or two amino acid substitutions in the group of interface amino acids consisting of residues 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596 of SEQ ID NO:2 or an equivalent position in a parent glucoamylase.

### Embodiment 10

The glucoamylase variant according to any one of embodiments 1-9 comprising one, two or three amino acid substitutions in the group of catalytic core amino acids consisting of residues not in direct contact with the starch binding domain in positions 1 to 484 with exception of position 24, 26, 27, 29, 30, 40, 42, 43, 44, 46, 48, 49, 110, 111, 112, 114, 116, 117, 118 and 119 of SEQ ID NO: 2, or an equivalent position in a parent glucoamylase.

### Embodiment 11

The glucoamylase variant according to any one of embodiments 1-10 having and RDF of at least 74.5%.

### Embodiment 12

The glucoamylase variant according to any one of embodiments 1-11, wherein the glucoamylase variant has at least 80% sequence identity with SEQ ID NO: 1, 2, 13, 18, 19, 20, 21, or 22.

### Embodiment 13

The glucoamylase variant according to any one of embodiments 1-12, wherein the glucoamylase variant has at least 85% sequence identity with SEQ ID NO: 1, 2, 13, 18, 19, 20, 21, or 22.

### Embodiment 14

The glucoamylase variant according to any one of embodiments 1-13, wherein the glucoamylase variant has at least 90% sequence identity with SEQ ID NO: 1, 2, 13, 18, 19, 20, 21, or 22.

### Embodiment 15

The glucoamylase variant according to any one of embodiments 1-14, wherein the glucoamylase variant has at least 95% sequence identity with SEQ ID NO: 1, 2, 13, 18, 19, 20, 21, or 22.

### Embodiment 16

The glucoamylase variant according to any one of embodiments 1-15, wherein the glucoamylase variant has at least 99.5% sequence identity with SEQ ID NO: 1, 2, 13, 18, 19, 20, 21, or 22.

### Embodiment 17

The glucoamylase variant according to any one of embodiments 1-16, wherein said glucoamylase variant has at least 80% sequence identity such as at least 85%, 90%, 95%, or 99.5% sequence identity with SEQ ID NO: 2 or 13.

### Embodiment 18

The glucoamylase variant according to any one of embodiments 1-17 consisting of the parent sequence of the amino acids of SEQ ID NO: 1, 2, 13, 18, 19, 20, 21, or 22, which sequence of amino acids has one or two amino acid substitutions in the group of interface amino acids consisting of residues F29, I43, Y48, F116 and H502 of SEQ ID NO: 2, wherein the substitution in I43 is I43Q, and the substitution in Y48 is Y48V, or an equivalent position in the parent glucoamylase; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W, or an equivalent position in the parent glucoamylase.

### Embodiment 19

The glucoamylase variant according to any one of embodiments 1-18 consisting of the sequence of the amino acids of SEQ ID NO: 2, which sequence of amino acids has one or two amino acid substitutions in the group of interface amino acids consisting of residues F29, I43, Y48, F116 and H502 of SEQ ID NO: 2, wherein the substitution in I43 is I43Q, and the substitution in Y48 is Y48V; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W.

### Embodiment 20

The glucoamylase variant according to any one of embodiments 1-19 consisting of the sequence of the amino acids of SEQ ID NO: 13, which sequence of amino acids has one or two amino acid substitutions in the group of interface amino acids consisting of residues F29, I43, Y48, F116 and H502 of SEQ ID NO: 13, wherein the substitution in 143 is I43Q, and the substitution in Y48 is Y48V; and one, two or three amino acid substitutions in the group of catalytic core amino acid residues consisting of residues S97, L98, Y147, F175, G483 and T484 of SEQ ID NO: 13, wherein the substitution in S97 is S97M, the substitution in G483 is G483S and the substitution in T484 is T484W SEQ ID NO: 13.

### Embodiment 21

The glucoamylase variant according to any one of embodiments 1-20, wherein the glucoamylase variant exhibits decreased thermostability as compared to the parent glucoamylase.

### Embodiment 22

The glucoamylase variant according to any one of embodiments 1-21, which glucoamylase variant is inactivated by pasteurisation such as using less than 16.8, 16, 15, 14, 13, 12, 11, 10, 9, 8, 7, 6, 5, or 4 pasteurisation units (PU) in beer.

### Embodiment 23

The glucoamylase variant according to any one of embodiments 1-22, which glucoamylase variant when in its crystal form has a crystal structure for which the atomic coordinates of the main chain atoms have a root-mean-square deviation from the atomic coordinates of the equivalent main chain atoms of TrGA (as defined in Table 20 in WO2009/067218) of less than 0.13 nm following alignment of equivalent main chain atoms, and which have a linker region, a starch binding domain and a catalytic domain.

### Embodiment 24

The glucoamylase variant according to any one of embodiments 1-23 comprising one or two amino acid substitutions in the group of interface amino acids consisting of residues F29, I43, Y48, F116 and H502 of SEQ ID NO: 2, wherein the substitution in I43 is I43Q, and the substitution in Y48 is Y48V, or an equivalent position in the parent glucoamylase; and one, two or three amino acid substitutions in the group of catalytic core amino acid residues consisting of residues S97, L98, Y147, F175, G483 and T484 of SEQ ID NO: 2, wherein the substitution in S97 is S97M, the substitution in G483 is G483S and the substitution in T484 is T484W, or an equivalent position in the parent glucoamylase

### Embodiment 25

The glucoamylase variant according to any one of the embodiments 1-24 comprising an amino acid substitution at the residue corresponding to position F29 of SEQ ID NO:2 or an equivalent position in a parent glucoamylase.

### Embodiment 26

The glucoamylase variant according to any one of the embodiments 1-25 comprising the following amino acid substitution F29A/R/N/D/C/E/F/G/H/K/S/T/Q/I/L/M/P/V of SEQ ID NO:2, or an equivalent position in a parent glucoamylase.

### Embodiment 27

The glucoamylase variant according to any one of the embodiments 1-26 comprising the following amino acid substitution F29V of SEQ ID NO:2, or an equivalent position in a parent glucoamylase.

### Embodiment 28

The glucoamylase variant according to any one of the embodiments 1-27 comprising an amino acid substitution at the residue corresponding to position 143 of SEQ ID NO:2, or an equivalent position in a parent glucoamylase.

### Embodiment 29

The glucoamylase variant according to any one of the embodiments 1-28 comprising the following amino acid substitution I43Q of SEQ ID NO:2, or an equivalent position in a parent glucoamylase.

### Embodiment 30

The glucoamylase variant according to any one of the embodiments 1-29 comprising an amino acid substitution at the residue corresponding to position Y48 of SEQ ID NO:2, or an equivalent position in a parent glucoamylase.

### Embodiment 31

The glucoamylase variant according to any one of the embodiments 1-30 comprising the following amino acid substitution Y48V of SEQ ID NO:2, or an equivalent position in a parent glucoamylase.

### Embodiment 32

The glucoamylase variant according to any one of the embodiments 1-31 comprising an amino acid substitution at the residue corresponding to position F116 of SEQ ID NO:2, or an equivalent position in a parent glucoamylase.

### Embodiment 33

The glucoamylase variant according to any one of the embodiments 1-32 comprising the following amino acid substitution F116M of SEQ ID NO:2, or an equivalent position in a parent glucoamylase.

### Embodiment 34

The glucoamylase variant according to any one of the embodiments 1-33 comprising an amino acid substitution at the residue corresponding to position H502 of SEQ ID NO:2, or an equivalent position in a parent glucoamylase.

### Embodiment 35

The glucoamylase variant according to any one of the embodiments 1-34 comprising the following amino acid substitution H502A/N/D/C/E/F/G/H/K/S/T/Q/I/L/M/P/V/W/Y of SEQ ID NO:2, or an equivalent position in a parent glucoamylase.

### Embodiment 36

The glucoamylase variant according to any one of the embodiments 1-35 comprising the following amino acid substitution H502S/E of SEQ ID NO:2, or an equivalent position in a parent glucoamylase.

### Embodiment 37

The glucoamylase variant according to any one of the embodiments 1-36 comprising an amino acid substitution at the residue corresponding to position S97 of SEQ ID NO:2, or an equivalent position in a parent glucoamylase.

### Embodiment 38

The glucoamylase variant according to any one of the embodiments 1-37 comprising the following amino acid substitution S97M of SEQ ID NO:2, or an equivalent position in a parent glucoamylase.

### Embodiment 39

The glucoamylase variant according to any one of the embodiments 1-38 comprising an amino acid substitution at the residue corresponding to position L98 of SEQ ID NO:2, or an equivalent position in a parent glucoamylase.

### Embodiment 40

The glucoamylase variant according to any one of the embodiments 1-39 comprising the following amino acid substitution L98A/R/N/E/G/H/K/S/T/Q/I/L/M/P/V/Y of SEQ ID NO:2, or an equivalent position in a parent glucoamylase.

### Embodiment 41

The glucoamylase variant according to any one of the embodiments 1-40 comprising the following amino acid substitution L98E of SEQ ID NO:2, or an equivalent position in a parent glucoamylase.

### Embodiment 42

The glucoamylase variant according to any one of the embodiments 1-41 comprising an amino acid substitution at the residue corresponding to position Y147 of SEQ ID NO:2, or an equivalent position in a parent glucoamylase.

### Embodiment 43

The glucoamylase variant according to any one of the embodiments 1-42 comprising the following amino acid substitution Y147R of SEQ ID NO:2, or an equivalent position in a parent glucoamylase.

### Embodiment 44

The glucoamylase variant according to any one of the embodiments 1-43 comprising an amino acid substitution at the residue corresponding to position F175 of SEQ ID NO:2, or an equivalent position in a parent glucoamylase.

### Embodiment 45

The glucoamylase variant according to any one of the embodiments 1-44 comprising the following amino acid substitution F175V/I/L of SEQ ID NO:2, or an equivalent position in a parent glucoamylase.

### Embodiment 46

The glucoamylase variant according to any one of the embodiments 1-45 comprising an amino acid substitution at the residue corresponding to position G483 of SEQ ID NO:2, or an equivalent position in a parent glucoamylase.

### Embodiment 47

The glucoamylase variant according to any one of the embodiments 1-46 comprising the following amino acid substitution G483S of SEQ ID NO:2, or an equivalent position in a parent glucoamylase.

### Embodiment 48

The glucoamylase variant according to any one of the embodiments 1-47 comprising an amino acid substitution at the residue corresponding to position T484 of SEQ ID NO:2, or an equivalent position in a parent glucoamylase.

### Embodiment 49

The glucoamylase variant according to any one of the embodiments 1-48 comprising the following amino acid substitution T484W of SEQ ID NO:2, or an equivalent position in a parent glucoamylase.

### Embodiment 50

The glucoamylase variant according to any one of the embodiments 1-49, wherein the total number of amino acid substitutions
(1) in the group of interface amino acid residue consisting of residues 24, 26, 27, 29, 30, 40, 42, 43, 44, 46, 48, 49, 110, 111, 112, 114, 116, 117, 118, 119, 500, 502, 504, 534, 536, 537, 539, 541, 542, 543, 544, 546, 547, 548, 580, 583, 585, 587, 588, 589, 590, 591, 592, 594, and 596 of SEQ ID NO:2 or an equivalent position in a parent glucoamylase; and
(2) in the group of catalytic core amino acid consisting of residues not in direct contact with the starch binding domain in positions 1 to 484 with exception of position 24, 26, 27, 29, 30, 40, 42, 43, 44, 46, 48, 49, 110, 111, 112, 114, 116, 117, 118 and 119 of SEQ ID NO: 2, or an equivalent position in a parent glucoamylase; are two, three or four.

### Embodiment 51

The glucoamylase variant according to any one of embodiments 1-50 comprising the following amino acid substitutions F29V-G483S, Y48V-L98E-H502S, F116M-F175V, F175V-H502E, I43Q-F175I-H502S, I43Q-F175I, F29V-597M-G483S-T484W, or L98E-Y147R-H502S of SEQ ID NO: 2, or an equivalent position in a parent glucoamylase.

### Embodiment 52

The glucoamylase variant according to any one of embodiments 1-51 further comprising the following amino acid substitutions L417V, T430A, Q511H, A539R and N563I.

### Embodiment 53

The glucoamylase variant according to any one of embodiments 1-52 consisting of SEQ ID NO: 14.

### Embodiment 54

The glucoamylase variant according to any one of embodiments 1-52 consisting of SEQ ID NO: 15.

### Embodiment 55

The glucoamylase variant according to any one of embodiments 1-52 consisting of SEQ ID NO: 16.

### Embodiment 56

The glucoamylase variant according to any one of embodiments 1-52 consisting of SEQ ID NO: 17.

### Embodiment 57

The glucoamylase variant according to any one of embodiments 1-56, wherein the parent glucoamylase has a catalytic domain that has at least 80% sequence identity with SEQ ID NO: 1, 2, 13, 18, 19, 20, 21, and/or 22.

### Embodiment 58

The glucoamylase variant according to any one of embodiments 1-57, wherein the parent glucoamylase has a starch binding domain that has at least 80% sequence identity with SEQ ID NO: 11, 24, 25, 26, 27, 28, and/or 29.

### Embodiment 59

The glucoamylase variant according to any one of embodiments 1-58, wherein the parent glucoamylase is selected from a glucoamylase obtained from a Trichoderma spp., an Aspergillus spp., a Humicola spp., a Penicillium spp., a Talaromyces spp., or a Schizosaccharmyces spp.

### Embodiment 60

The glucoamylase variant of embodiment 59, wherein the parent glucoamylase is obtained from a Trichoderma spp. or an Aspergillus spp.

### Embodiment 61

The glucoamylase variant according to any one of embodiments 1-60, wherein the glucoamylase variant exhibits altered thermostability as compared to the parent glucoamylase.

### Embodiment 62

The glucoamylase variant according to embodiment 61, wherein the altered thermostability is a decreased thermostability.

### Embodiment 63

The glucoamylase variant according to any one of embodiments 1-62, wherein the glucoamylase variant exhibits altered specific activity as compared to the parent glucoamylase.

### Embodiment 64

The glucoamylase variant according to embodiment 63, wherein the altered specific activity is similar or increased specific activity.

### Embodiment 65

The glucoamylase variant according to any one of embodiments 1-64, wherein the glucoamylase variant exhibits both decreased thermostability and similar or increased specific activity as compared to the parent glucoamylase.

### Embodiment 66

The glucoamylase variant according to any one of embodiments 1-65, wherein the percentage of identity of one amino acid sequence with, or to, another amino acid sequence is determined by the use of the protein-protein Blast search (http://blast.ncbi.nlm.nih.gov) with default settings: score matrix: blosum62, non-redundant protein sequences database and the blast algorithm

| | | |
|---|---|---|
| Settings | Expect threshold | 10 |
| | Max matches in a query range | 0 |
| | Gap opening penalty | 11 |
| | Gap extension penalty | 1 |
| | Compositional adjustment: | |
| | Conditional compositional score | |
| | matrix adjustment | |
| | Mask and filters | No |

### Embodiment 67

The glucoamylase variant according to any one of embodiments 1-66, which glucoamylase variant is inactivated by pasteurisation such as using less than 16.8, 16, 15, 14, 13, 12, 11, 10, 9, 8, 7, 6, 5, or 4 pasteurisation units (PU) in beer.

### Embodiment 68

The glucoamylase variant according to any one of embodiments 1-67, which glucoamylase variant has a glucoamylase activity (GAU) of 0.05-10 GAU/mg, such as 0.1-5 GAU/mg, such as 0.5-4 GAU/mg, such as 0.7-4 GAU/mg, or such as 2-4 GAU/mg.

### Embodiment 69

The glucoamylase variant according to any one of the embodiments 1-68, which is obtained by recombinant expression in a host cell.

### Embodiment 70

A nucleic acid capable of encoding a glucoamylase variant according to any one of embodiments 1-69.

### Embodiment 71

An expression vector or plasmid comprising a nucleic acid according to embodiment 70, or capable of expressing a glucoamylase variant according to any one of embodiments 1-69.

### Embodiment 72

The expression vector or plasmid according to embodiment 71 comprising a promoter derived from Trichoderma such as a T. reesei cbhI-derived promoter.

### Embodiment 73

The expression vector or plasmid according to any one of embodiments 71-72 comprising a terminator derived from Trichoderma such as a T. reesei cbhI-derived terminator.

### Embodiment 74

The expression vector or plasmid according to any one of embodiments 71-73 comprising one or more selective markers such as Aspergillus nidulans amdS and pyrG.

### Embodiment 75

The expression vector or plasmid according to any one of embodiments 71-74 comprising one or more telomere regions allowing for a non-chromosomal plasmid maintenance in a host cell.

### Embodiment 76

A host cell having heterologous expression of a glucoamylase variant as defined in any one of embodiments 1-69.

### Embodiment 77

The host cell according to embodiment 76, wherein the host cell is a fungal cell.

### Embodiment 78

The host cell according to embodiment 77, wherein the fungal cell is of the genus Trichoderma.

### Embodiment 79

The host cell according to embodiment 78, wherein the fungal cell is of the species Trichoderma reesei.

### Embodiment 80

The host cell according to embodiment 77, wherein the fungal cell is of the species Hypocrea jecorina.

### Embodiment 81

A host cell comprising, preferably transformed with, a plasmid or an expression vector as defined in any one of embodiments 71-75.

### Embodiment 82

A method of isolating a glucoamylase variant as defined in any one of embodiments 1-69, the method comprising the steps of inducing synthesis of the glucoamylase variant in a host cell as defined in any one of embodiments 76-81 having heterologous expression of said glucoamylase variant and recovering extracellular protein secreted by said host cell, and optionally purifying the glucoamylase variant.

### Embodiment 83

A method for producing a glucoamylase variant as defined in any one of embodiments 1-69, the method comprising the steps of inducing synthesis of the glucoamylase variant in a host cell as defined in any one of embodiments 76-81 having heterologous expression of said glucoamylase variant, and optionally purifying the glucoamylase variant.

### Embodiment 84

A method of expressing a glucoamylase variant as defined in any one of embodiments 1-69, the method comprising obtaining a host cell as defined in any one of embodiments 76-81 and expressing the glucoamylase variant from said host cell, and optionally purifying the glucoamylase variant.

### Embodiment 85

The method according to any one of embodiments 82-84, wherein the glucoamylase variant as defined in any one of embodiments 1-69 is the dominant secreted protein.

### Embodiment 86

A composition comprising one or more glucoamylase variant(s) as defined in any one of embodiments 1-69.

### Embodiment 87

The composition according to embodiment 86, wherein the composition is selected from among a starch hydrolyzing composition, a saccharifying composition, a detergent composition, an alcohol fermentation enzymatic composition, and an animal feed animal feed composition.

### Embodiment 88

The composition according to any one of embodiments 86-87, comprising one or more further enzyme(s).

### Embodiment 89

The composition according to embodiment 88, wherein the one or more further enzyme(s) is selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

### Embodiment 90

The composition according to any one of embodiments 86-89, which glucoamylase variant(s) and/or one or more further enzyme(s) is inactivated by pasteurisation.

### Embodiment 91

The composition according to embodiment 90, wherein the glucoamylase variant and/or the one or more further enzyme(s) is inactivated by pasteurisation such as by using less than 50, 45, 40, 35, 30, 25, 24, 23, 22, 21, 20, 19, 18, 17, 16, or 15 pasteurisation units (PU) in beer.

### Embodiment 92

Use of a glucoamylase variant as defined in any one of embodiments 1-69 or a composition as defined in any one of embodiments 86-91 in a fermentation, wherein said glucoamylase variant or composition is added before or during a fermentation step.

### Embodiment 93

The use according to embodiment 92, wherein said fermentation step, and optional beer filtration step, is followed by a pasteurisation step.

### Embodiment 94

The use according to any one of embodiments 92-93, wherein said fermentation is comprised in a process for making a fermented beverage.

### Embodiment 95

The use according to any one of embodiments 92-94, wherein said fermented beverage is selected from the group consisting of beer such as low alcohol beer or low calorie beer.

### Embodiment 96

The use according to any one of embodiments 92-95, wherein said glucoamylase variant or said composition is added in combination with one or more further enzyme(s).

### Embodiment 97

The use according to embodiment 96, wherein said one or more further enzyme(s) is selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

### Embodiment 98

The use according to any one of embodiments 92-97, wherein the glucoamylase variant and/or the one or more further enzyme(s) is inactivated in the pasteurisation step.

### Embodiment 99

The use according to any one of embodiments 92-98, wherein the glucoamylase variant is added in an amount of for example 0.01-50 mg pr. ml fermented wort, such as 0.05-25 mg pr. ml fermented wort, such as 0.1-15 mg pr. ml fermented wort, such as 0.2-10 mg pr. ml fermented wort, such as 1-5 mg pr. ml fermented wort.

### Embodiment 100

Use of a thermolabile glucoamylase variant to enhance the production of fermentable sugars in the fermentation step of a brewing process, wherein the glucoamylase variant is as defined in any one of embodiments 1-69.

### Embodiment 101

A method which comprises adding a glucoamylase variant as defined in any one of embodiments 1-69 or a composition as defined in any one of embodiments 86-91 before or during a fermentation step, such as a fermentation step with yeast.

### Embodiment 102

The method according to embodiment 101 comprising a pasteurisation step after the fermentation step or optional beer filtration step.

### Embodiment 103

The method according to any one of embodiments 101-102, wherein said fermentation is comprised in a process for making a fermented beverage.

### Embodiment 104

The method according to any one of embodiments 101-103, wherein said fermented beverage is selected from the group consisting of beer such as low alcohol beer, low calorie beer.

### Embodiment 105

The method according to any one of embodiments 101-104, wherein said glucoamylase variant or said composition is added in combination with one or more further enzyme(s).

### Embodiment 106

The method according to embodiment 105, wherein said one or more further enzyme(s) is selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

### Embodiment 107

The method according to any one of embodiments 101-106, wherein the glucoamylase variant and/or the one or more further enzyme(s) is inactivated in the pasteurisation step.

### Embodiment 108

The method according to any one of embodiments 101-107, wherein the glucoamylase variant is added in an amount of for example 0.01-50 mg pr. ml fermented wort, such as 0.05-25 mg pr. ml fermented wort, such as 0.1-15 mg pr. ml fermented wort, such as 0.2-10 mg pr. ml fermented wort, such as 1-5 mg pr. ml fermented wort.

### Embodiment 109

The method according to any one of embodiments 101-108 for production of a fermented beverage which comprises the following steps:

a) preparing a mash,
b) filtering the mash to obtain a wort, and
c) fermenting the wort to obtain a fermented beverage,

### Embodiment 110

The method according to embodiment 109, wherein a glucoamylase variant as defined in any one of embodiments 1-69 or a composition as defined in any one of embodiments 86-91 is added to:

the mash of step (a) and/or
the wort of step (b) and/or
the wort of step (c).

### Embodiment 111

The method according to embodiment 109 or 110, wherein the fermented beverage is subjected to a pasteurisation step (d).

### Embodiment 112

The method according to any one of embodiments 109-111, wherein the mash in step (a) is obtained from a grist.

### Embodiment 113

The method according to embodiment 112, wherein the grist comprises one or more of malted and/or unmalted grain, or starch-based material from another crop.

### Embodiment 114

The method according to any one of embodiments 109-113, further comprising contacting the mash of step (a) with one or more further enzyme(s).

### Embodiment 115

The method according to embodiment 114, wherein the enzyme is selected among a starch debranching enzyme, R-enzyme, limit dextrinase, alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

### Embodiment 116

The method according to any one of embodiments 109-115, further comprising contacting the wort of step (b) or (c) with one or more further enzyme(s), wherein the enzyme is selected among a starch debranching enzyme, isoamylase and limit dextrinase, including any combinations thereof.

### Embodiment 117

A fermented beverage wherein the fermented beverage is produced by a method as defined in any one of embodiments 109-116.

### Embodiment 118

The fermented beverage according to embodiment 117, which is beer such as low alcohol beer or low calorie beer.

### Embodiment 119

A method for the production of a food, feed, or beverage product, such as an alcoholic or non-alcoholic beverage, such as a cereal- or malt-based beverage like beer or whiskey, such as wine, cider, vinegar, rice wine, soya sauce, or juice, said method comprising the step of treating a starch and/or sugar containing plant material with a glucoamylase variant according to embodiments 1-69, or a composition as defined in any one of embodiments 86-91.

### Embodiment 120

A kit comprising a glucoamylase variant according to any one of embodiments 1-69, or a composition as defined in any one of embodiments 86-91; and instructions for use of said glucoamylase variant or composition.

### Embodiment 121

Use of a glucoamylase variant according to any one of embodiments 1-69, or a composition according to any one of embodiments 86-91 and 117-118, in the production of a first- or second-generation biofuel, such as bioethanol and/or biobutanol.

### Embodiment 122

Use of a glucoamylase variant according to any one of embodiments 1-69, or a composition according to any one of embodiments 86-91 and 117-118, in the production of a biochemical, such as bio-based isoprene.

### Embodiment 123

Method for the production of a first- or second-generation biofuel, such as bioethanol and/or biobutanol, said method comprising the step of treating a starch comprising material with a glucoamylase variant according to any one of embodiments 1-69, or a composition according to any one of embodiments 86-91 and 117-118.

### Embodiment 124

Method for the production of a biochemical, such as bio-based isoprene, said method comprising the step of treating a starch comprising material with a glucoamylase variant according to any one of embodiments 1-69, or a composition according to any one of embodiments 86-91 and 117-118.

### Embodiment 125

A glucoamylase variant obtained by the method according to any one of embodiments 82-85.

### Embodiment 126

A composition comprising the product according to embodiment 125, such as wherein the product is in a range of 0.1%-99.9%.

The following examples are provided and it should be understood that the various modifications can be made without departing from the spirit of the embodiments discussed.

### EXAMPLES

### Assays and Methods

The following assays and methods are used in the examples provided below. The methods used to provide variants are described below. However, it should be noted that different methods may be used to provide variants of a parent enzyme and the invention is not limited to the methods used in the examples. It is intended that any suitable means for making variants and selection of variants may be used.

### Production of GA by Fermentation

400× Trace Element Solution:

Dilute in 1000 ml of demi water: Anhydrous Citric Acid (175 g), FeSO₄*7 H₂O (200 g), ZnSO₄*7 H₂O (16 g), CuSO₄*5 H₂O (3.2 g), MnSO₄*H₂O (1.4 g), H₃BO₃ (0.8 g). It may be helpful to acidify this to get all components into solution. The solution was filtered and sterilized.

LD-Medium:

Add to ˜800 ml of demi water: Casamino acids (9 g), MgSO₄*7H₂O (1 g), (NH₄)₂SO₄ (5 g), KH₂PO₄ (4.5 g), CaCl₂*2H₂O (1 g), Piperazine-1,4-bis-propanesulfonic acid (PIPPS) buffer (33 g), 400×T. reesei trace elements (2.5 ml), Adjust pH to 5.5 with NaOH 4N. Adjust final volume to 920 ml.

2×Amd S Base Ager (1 Litre):

Mix KH₂PO₄ (30 g), 1M Acetamide (20 ml), 1M CsCl (20 ml), 20% MgSO4.7H₂O (6 ml), 20% CaCl₂.2H₂O (6 ml), T. reesei spore elements 400× (2 ml), 50% glucose.H₂O (80 ml). Adjust pH to 4.5 with 4N NaOH Make up to 1 L and filter sterilize. Store at 4° C.

Initial Culture:

Trichoderma reesei strains were grown on AmdS-Base agar plates. To produce agar plates minimal media agar was boiled and after cooling down to app. 50° C. it was diluted with 2×AmdS Base 1:1 and poured on petri dishes. After sporulation (app. 6-7 days) the plates were scraped with 2 ml saline 0.015% Tween 80. Approx 1 ml was added to glycerol tubes containing 500-600 μl 35% glycerol and stored at −80° C. The pre-culture fermentations were started directly from this spore suspension.

Pre Culture:

The medium is made by adding 2.5% glucose to the LD-medium, which is subsequently made up to 1 L. To produce biomass 50 μl spore suspension is added to 100 ml medium (sterilised in 500 ml shake flask). The flasks are incubated on a rotary shaker at 30° C., 180 rpm for 2 days, then 10 ml suspension is used to inoculate a new baffled shake flask, which is incubated under similar conditions for 1 day. The content of this flask is used to inoculate a fermentor. Alternatively fermentation of the pre-culture was initiated by a piece (˜1 cm²) of a fresh PDA plate with T. reesei.

Main Culture:

To make 1 L of medium, 40 ml glucose/sophorose mix (Danisco, Jamsa, Finland) was added to the LD-medium and mede up to 1 L. 6 L fermentors containing 4 L of medium were inoculated with the pre-culture, and grown at pH 3.5 for approximately 16 hours at 34° C., until CER/OUR (Carbondioxide Excretion Rate/Oxygen Uptake Rate) started falling. Then temperature was lowered to 28° C., pH was raised to 5.5 and the fermentation was continued for approximately 80 hours. The cell culture is harvested and media clarified by centrifugation (4000 rpm at 25 min.) and filtration (VacuCap 90, 0.2 μm). Following, the ferment was concentrated and stored at −20° C.

### Purification of TrGA Variants

Culture supernatants of expressed TrGA variants were purified in one step by affinity chromatography using a BioRAD DUO-Flow FPLC system (BioRAD, U.S.). Chromatography was carried out manually on a BioRAD FPLC system. A 15 ml β-cyclodextrin column was made by immobilizing β-cyclodextrin (Sigma-Aldrich Zwijndrecht, The Netherlands; CAS nr:68168-23-0) on Epoxy-activated Sepharose™ 6 B (GE Healthcare, Diegem, Belgium; Lot: 10021987). This β-CD-column was equilibrated with Buffer A at a flow rate of 2 ml/min. This flow rate was maintained throughout the purification. The sample containing 500 GAU units was loaded onto the column through the inlet tubing and fractions of 10 ml were collected throughout purification. The flowthrough was discarded and the buffer was switched to 100% Buffer B (10 mM a-cyclodextrin in 25 mM Na-acetat pH 4.3 (Sigma, 28705)) after stabilisation of the baseline by extensive washing with Buffer A. Bound TrGA variants was eluted from the column and the buffer was finally switched back to buffer A after all protein was eluted. Eluted protein was desalted to remove a-cyclodextrin and analyzed for glucoamylase activity and by sodium dodecyl sulfate-polyacrylamide gel electrophoresis (SDS-PAGE).

### Protein Quantification of Purified TrGA Variants

A Bradford assay was used for total protein quantification. The reagent solution was Bradford Quikstart work solution (BioRad cat#500-0205). 100 μl of supernatant was placed in a fresh 96-well flat bottom plate. To each well 200 UI reagent was added and incubated for 5 minutes at room temperature. The absorbance was measured at 595 nm in a MTP-reader (Molecular Devices Spectramax 190). Protein concentrations were calculated according to a Bovine Serum Albumin (BSA) (0-50 Ug/ml) standard curve.

### Gel Electrophoresis Analysis

All sodium dodecyl sulfate polyacrylamide gel electrophoresis (SDS-PAGE) was run with the Invitrogen NuPAGE® Novex 4-12% Bis-Tris Gel 1.0 mm, 12 well (Cat# NP0321box), Novex See-Blue® Plus2 prestained Standard (Cat# LC5925), Invitrogen Simply Blue Safestain (Cat#LC6060) and NuPAGE® MES SDS Running Buffer (Cat# NP0002) according to the manufacturer's protocol.

pNPG Glucoamylase Activity Assay for 96-Well Microtiter Plates

The reagent solutions were: NaAc buffer: 200 mM sodium acetate buffer pH 4.5; Substrate: 50 mM p-nitrophenyl-α-D-glucopyranoside (Sigma N-1377) in NaAc buffer (0.3 g/20 ml) and stop solution: 800 mM glycine-NaOH buffer pH 10. 30 μl filtered supernatant was placed in a fresh 96-well flat bottom micro titer plate (MTP). To each well 50 μl NaAc buffer and 120 μl substrate was added and incubated for 30 minutes at 50° C. (Thermolab systems iEMS Incubator/shaker HT). The reaction was terminated by adding 100 μl stop solution. The absorbance was measured at 405 nm in a MTP-reader (Molecular Devices Spectramax 384 plus) and the activity was calculated using a molar extinction coefficient of 0.011 μM/cm.

### Determination of GAU Activity in 96-Well Microtiter Plates

Specific chromogenic glucoamylase assay with a pNP-β-maltoside substrate and expressed as the amount of p-nitrophenol that is produced from the substrate under defined assay conditions. The specific substrate p-nitrophenyl-β-maltoside is not hydrolyzed by α-amylase, α-glucosidase, and transglucosidase, which may appear as contaminants in commercial glucoamylase preparations

Substrate:

p-Nitrophenyl-β-maltoside (4 mM), plus thermostable β-glucosidase (5 U/ml) (from assay R-AMGR3 05/04; Megazyme International Wicklow, Ireland) was freshly prepared.

Buffer:

200 mM Sodium acetate buffer (pH 4.5).

Enzyme samples were diluted by at least a factor 10 in sodium acetate buffer In a 96 well plate: 20 μL substrate was mixed with 20 μL enzyme solution and incubate at 40° C. with agitation for 10 minutes. 300 μL 2% Trizma base was added to terminate reaction and develop the colour. Absorbance at 400 nm was measured against a reagent blank.

Blanks were prepared by adding 300 μL of Trizma base solution (2%) to 20 μL of substrate with vigorous stirring, followed by the enzyme solution (20 μL). Activity was calculated as follows:

$$Activity ( GAU / mL ) = \frac{Δ {A}_{400}}{10} \cdot \frac{340}{20} \cdot \frac{1}{18.1} \cdot \frac{1}{0 , 88} \cdot Dilution$$

Where: GAU=International units of enzyme activity. One Unit is the amount of enzyme which releases one μmole of p-nitrophenol from the substrate per minute at the defined pH and temperature. ΔA₄₀₀=absorbance (reaction)−Absorbance (blank). 10=incubation time (min). 340=final reaction volume (μL). 20=volume of enzyme assayed (μL) 18.1=E mM p-nitrophenol in 2% trizma base (pH ˜8.5) at 400 nm (unit: μM⁻¹*cm⁻¹). 0.88=Light path (cm).

### Thermal Stability Assay

The relative loss of glucoamylase activity was determined in degassed beer or NaAcetate buffer pH 4.5 in a lab-scale pasteurisation assay. The sample was diluted 1:10 in beer or buffer and transferred to thin glass cuvette and placed in water bath at 72° C. where time and temperature were measured. Samples were withdrawn over time (0 to 100 sec) and hold on ice before determining the residual GAU activity. Dilution and mixing were performed in 96 well ELISA plates on a Biomek 3000 (Beckman Coulter). To measure enzyme thermostability under the conditions used in the present experiments, the GAU activity was determined before and after incubation of enzymes. Beer or buffer without glucoamylase was used as blank. The accumulated energy input was converted into pasteurisation units (PU), an energy equivalent index, by the equation stated below.

Pasteurisation units or PU refers to a quantitative measure of pasteurisation. One pasteurisation unit (1 PU) for beer is defined as a heat retention of one minute at 60 degrees Celsius. One calculates that:

PU=t×1.393̂(T−60), where:

t=time, in minutes, at the pasteurisation temperature in the pasteuriser
T=temperature, in degrees Celsius, in the pasteuriser
[̂(T−60) represents the exponent of (T−60)]

Thermostability was determined in regular degassed Pilsner (Royal Export Pilsner) pH (4.5) for the TrGA variants. Data is calculated as % relative activity as follows:

$$\frac{GAU\_residual - blank}{GAU\_initial - blank} \times 100 \%$$

Brew Analysis with Determination of Real Degree of Fermentation (RDF)

### Pure Malt Brew Analysis

340 g Munton's malt extract was dissolved in 1500 ml hot water. This slurry was added 5 pellets of Bitter hops from Hopfenveredlung, St. Johann: Alpha content of 16.0% (EBC 7.7 0 specific HPLC analysis), pH adjusted to 5.2 by H₂SO₄ and boiled for 1 hour before being autoclaved at 121° C. for 15 minutes, in order to destroy any residual glucoamylase activity and microbial contamination. At the end of mashing, the mashes were cooled, made up to 350 g and filtered. Filtrate volumes were measured after 30 minutes and the filtrated worts were sampled for specific gravity determination. The final wort was having an initial Specific Gravity of 1058.6 (i.e. 14.41° Plato). 60 ml of the wort were added to each 100 ml flask (Fermenting Vessel; FV), and then cooled 18° C. The enzymes were dosed on similar amount of protein (0.058 mg GA/mL wort) or similar β-D-maltoside activity (0.16 GAU/mL wort).

The following additions were made to the flasks:
Negative control flasks received 2 ml sterile water;
Positive control flasks received 2 ml of diluted DIAZYME® X4 (concentrated glucoamylase derived from a strain of Aspergillus niger) supplied by Genencor International; 2 ml of diluted the wild-type glucoamylase from Trichoderma reesei (TrGA wt) filtered fermentation broth; and 2 ml of diluted CS4 glucoamylase variant from Trichoderma reesei (TrGA CS4) filtered fermentation broth.
Test flasks received: 2 ml of a 3.5 mg→2 ml dilution of the thermolabile glucoamylase variants, equivalent to the same addition rate, in terms of amount (mg) of glucoamylase added per hl pitching wort, as that used for DIAZYME® X4 in the Positive control.

Each conical flask was dosed with W34/70 (Weihenstephan) freshly produced yeast at a dose rate of 0.6 g pr. 100 mL wort, the fermentation was allowed to proceed under standardised laboratory test conditions (an elevated temperature of 18.5° C., with gentle agitation of 150 rpm, in an orbital incubator for up to 88 hours). Each flask was analysed at scheduled intervals with respect to weight loss and specific gravity, while Real Degree of Fermentation (RDF, which is the Real Attenuation expressed in percentage form) was calculated for the final fermented wort (beer). Specific gravity of the wort before, during and after fermentation was measured using a specific gravity hydrometer or Anton-Paar density meter (e.g. DMA 4100 M) and Real Attenuation was calculated and expressed in percentage form as RDF according to the formulae listed by Ensminger (see http://hbd.org/ensmingr/ “Beer data: Alcohol, Calorie, and Attenuation Levels of Beer”). Monitoring weight loss during fermentation provides an indirect measure of CO2 evolution and hence ethanol formation.

Residual activity was measured before and after fermentation. Production of ethanol was indirectly measured by weight loss of ferments. Alcohol was measured on an Anton-Paar

### Malt-Adjunct Brew Analysis

A modified decoction mashing, using corn (maize) grist as adjunct was employed. The brewing protocol was modified from US 2009014247. 40% of the malt was substituted with corn (maize) grist with a moisture content of 12.6% (Benntag Nordic; Nordgetreide GmBH Lubec, Germany). All corn (maize) grist was heated to 100° C. at 2° C./min, together with 54% of the water and 5% of the malt (well modified Pilsner malt; Fuglsang Denmark). 5 min rests were held at 72° C. and 80° C. and a 10 min rest was held at 100° C. Hereafter the adjunct was cooled to 64° C. and combined with the main mash, also at 64° C. Enzymes were added at this stage, and the 64° C. rest was extended to 250 min. After fermentation the RDF values were determined.

Real degree of fermentation (RDF) value may be calculated according to the equation below:

$$R D F ( \% ) = ( 1 - \frac{R E}{° {P}_{initial}} ) \times 100$$

Where: RE=real extract=(0.1808×° Pᵢₙᵢₜᵢₐₗ)+(0.8192×° P_{final}), ° Pᵢₙᵢₜᵢₐₗ is the specific gravity of the standardised worts before fermentation and ° P_{final} is the specific gravity of the fermented worts expressed in degree plato.

In the present context, Real degree of fermentation (RDF) was determined from to the specific gravity and alcohol concentration.

Specific gravity and alcohol concentration was determined on the ferments using a Beer Alcolyzer Plus and a DMA 5000 Density meter (both from Anton Paar, Gratz, Austria). Based on these measurements, the real degree of fermentation (RDF) value was calculated according to the equation below:

$$R D F ( \% ) = \frac{O E - E ( r )}{O E} \times 100$$

Where: E(r) is the real extract in degree Plato (° P) and OE is the original extract in ° P.

### Example 1: Construction of TrGA Variants in the pTTT Vector for Expression in Trichoderma reesei

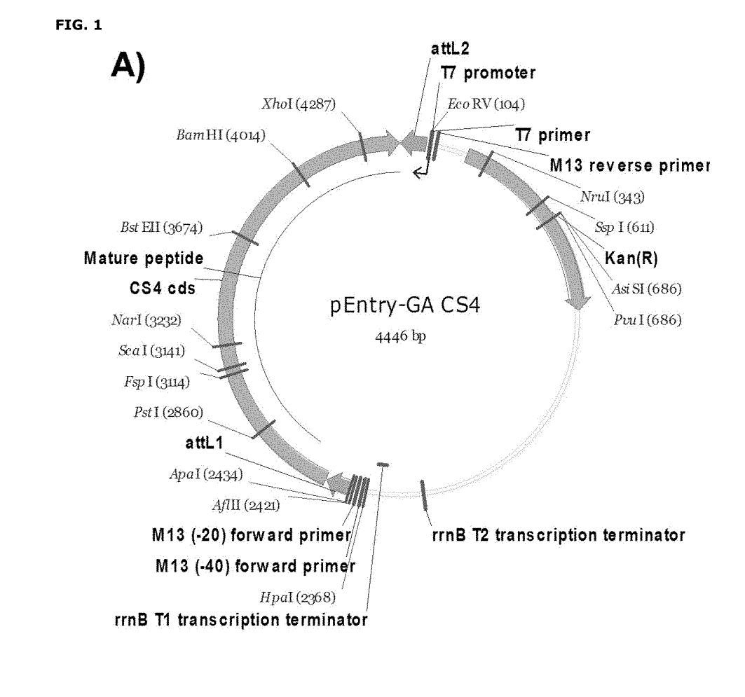

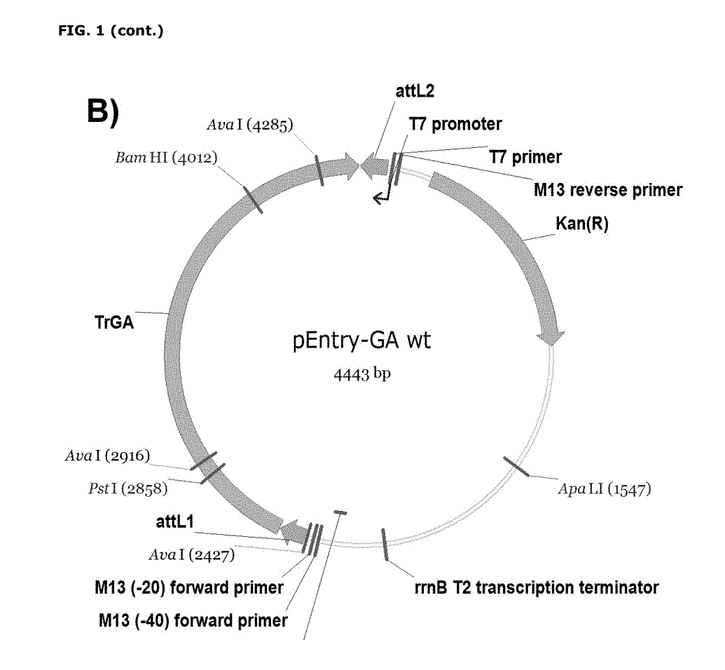

Hypocrea jecorina (anamorph Trichoderma reesei) optimized cDNA sequences (SEQ ID NO: 30 and SEQ ID NO:31) encoding TrGA wt and the TrGA CS4 variant, (SEQ ID NO:2 and SEQ ID NO:13), were cloned into pDONR™201 via the Gateway® BP recombination reaction (Invitrogen, Carlsbad, Calif., USA) resulting in the entry vector pEntry-CS4 and pEntry-GA (FIG. 1) as described in (US Patent Application no. US20110020899, US Patent Application no. US 20110014681). To enable the expression of the protein in H. jecorina, the TrGA CS4/GA wt coding sequence was cloned into the Gateway compatible destination vectors pTTT-pyrG13 or pTTT-pyr2 via the Gateway® LR recombination reaction.

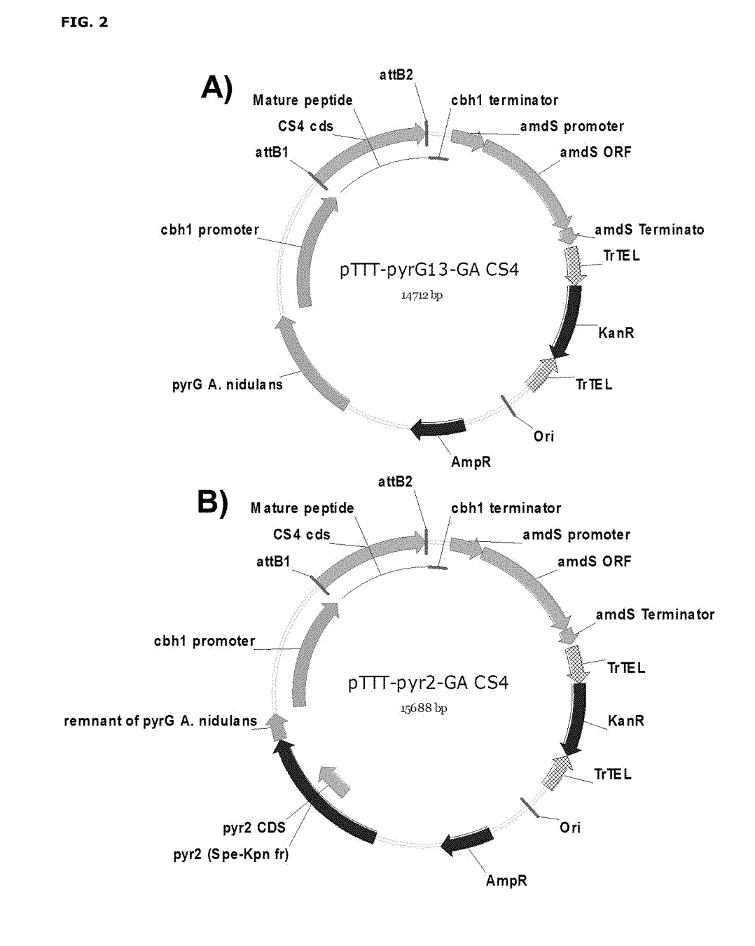

The pTTT-pyrG13 vector was described in WO2010141779A1. This vector contains the T. reesei cbhI-derived promoter and terminator regions allowing for a strong inducible expression of a gene of interest, the Aspergillus nidulans amdS and pyrG selective marker conferring growth of transformants on acetamide as a sole nitrogen source in the absence of uridine, and the T. reesei telomere regions allowing for non-chromosomal plasmid maintenance in a fungal cell. The cbhI promoter and terminator regions are separated by the chloramphenicol resistance gene, Cm^{R}, and the lethal E. coli gene, ccdB, flanked by the bacteriophage lambda-based specific recombination sites attR1, attR2. Such configuration allows for direct selection of recombinants containing the TrGA gene under the control of the cbhl regulatory elements in the right orientation via the Gateway® LR recombination reaction. The pTTT-pyr2 destination vector is a derivative of pTTT-pyrG13, where pyrG was replaced with the H. jecorina pyr2 gene conferring a H. jecorina uridine auxotroph ability to grow in the absence of uridine. The final expression vectors pTTT-pyrG13-GACS4 and pTTTpyr2-GACS4 are shown in FIG. 2.

The pEntry-CS4 and pEntry-GA wt plasmids were used as a template for combinatorial mutagenesis constructed by BASEClear (Leiden, The Netherlands). A request was made to the vendor for generation of specific single and combinatorial variants in the mature TrGA wt (SEQ ID NO. 2) and the mature TrGA CS4 variant (SEQ ID NO. 13) as shown in Table 1. The TrGA-CS4 variant include the following mutations L417V-T430A-Q511H-A539R-N563I compared to TrGA (wt).

TABLE 1
Mutations in TrGA (wt) and TrGA - CS4 variants

| No. | Samples ID | Backbone | Mutations |
|---|---|---|---|
| 1 | CPS3-B01 | TrGA - CS4 | T42M-I43Q-F175V-H502S |
| 2 | CPS2-F07 | TrGA - CS4 | I043Q-F175I-H502S |
| 3 | CPS2-A12 | TrGA - CS4 | F116M-F175V |
| 4 | CPS2-F05 | TrGA - CS4 | I043Q-F175I |
| 5 | CPS2-D11 | TrGA - CS4 | F175V-H502E |
| 6 | CPS2-F09 | TrGA - CS4 | T042L-F116M-F175I-H502E |
| 7 | CPS2-E08 | TrGA - CS4 | T042L-F175V-H502E |
| 8 | R_A_1 | TrGA - CS4 | F29V-G483S |
| 9 | R_A_2 | TrGA - CS4 | F29V-W156L-G483S |
| 10 | R_A_6 | TrGA | F29V-S97M-G483S-T484W |
| 11 | R_A_7 | TrGA | F29V-W156L-G483S |
| 12 | R_C_1 | TrGA - CS4 | Y48V-L98E-H502S |
| 13 | R_C_2 | TrGA - CS4 | Y48V-L98E-S102P-H502S |
| 14 | R_C_5 | TrGA - CS4 | Y48V-S102W-L111Q-F175V-A301I |
| 15 | R_C_7 | TrGA | Y48V-L98E-F175L-H502S |
| 16 | R_C_12 | TrGA - CS4 | F29Q-L98E-Y147R-A204T-T241L- |
| | | | N263E-H502S |
| 17 | R_C_13 | TrGA - CS4 | L98E-Y147R-H502S |
| 18 | R_C_22 | TrGA | Y48V-L98E-H502S |
| 19 | R_D_2 | TrGA - CS4 | H502S |
| 20 | R_D_3 | TrGA - CS4 | Y48V |
| 21 | R_D_5 | TrGA - CS4 | L98E |
| 22 | TrGA (wt) | TrGA | No |

### Example 2: Transformation of TrGA Variants into Trichoderma reesei

The TrGA variants were transformed into T. reesei using the PEG protoplast method. Plasmid DNAs confirmed by sequence analysis were provided by BASEClear (Leiden, The Netherlands) and transformed individually into a T. reesei host strain derived from RL-P37 bearing four gene deletions (Δcbh1, Δcbh2, Δegl1, Δegl2, i.e., “quad-deleted;” see U.S. Pat. No. 5,847,276, WO 92/06184, and WO 05/001036) using the PEG-Protoplast method (Penttila et al. (1987) Gene 61:155-164) with the following modifications.

For protoplast preparation, spores were grown for 16-24 hours at 24° C. in Trichoderma Minimal Medium (MM) (20 g/L glucose, 15 g/L KH₂PO₄, pH 4.5, 5 g/L (NH₄)₂SO₄, 0.6 g/L MgSO₄.7H₂O, 0.6 g/L CaCl₂.2H₂O, 1 ml of 1000×T. reesei Trace elements solution {5 g/L FeSO₄.7H₂O, 1.4 g/L ZnSO₄.7H₂O, 1.6 g/L MnSO₄.H₂O, 3.7 g/L CoCl₂.6H₂O}) with shaking at 150 rpm. Germinating spores were harvested by centrifugation and treated with 15 mg/ml of β-D-glucanase-G (Interspex—Art.No. 0439-1) solution to lyse the fungal cell walls. Further preparation of protoplasts was performed by a standard method, as described by Penttila et al. (1987 supra).

The transformation method was scaled down 10 fold. In general, transformation mixtures containing up to 600 ng of DNA and 1-5×10⁵ protoplasts in a total volume of 25 μl were treated with 200 ml of 25% PEG solution, diluted with 2 volumes of 1.2 M sorbitol solution, mixed with 3% selective top agarose MM with acetamide (the same Minimal Medium as mentioned above but (NH₄)₂SO₄ was substituted with 20 mM acetamide) and poured onto 2% selective agarose with acetamide either in 24 well microtiter plates or in a 20×20 cm Q-tray divided in 48 wells. The plates were incubated at 28° C. for 5 to 8 days. Spores from the total population of transformants regenerated on each individual well were harvested from the plates using a solution of 0.85% NaCl, 0.015% Tween 80. Spore suspensions were used to inoculate fermentations in 96 wells MTPs. In the case of 24 well MTPs, an additional plating step on a fresh 24 well MTP with selective acetamide MM was introduced in order to enrich the spore numbers.

### Example 3: Analysis of Enzyme Activity in Fermentation Broth of Glucoamylase Variants from Trichoderma reesei (TrGA)

The transformants were fermented, as described above in the Assays and Methods section and the supernatants containing the expressed variant TrGA proteins were tested for various properties.

In brief, mycelium was removed from the culture samples by centrifugation and the supernatant was analyzed for total protein content (BCA Protein Assay Kit, Pierce Cat. No. 23225) and GA activity, as described above in the Assays and Methods section.

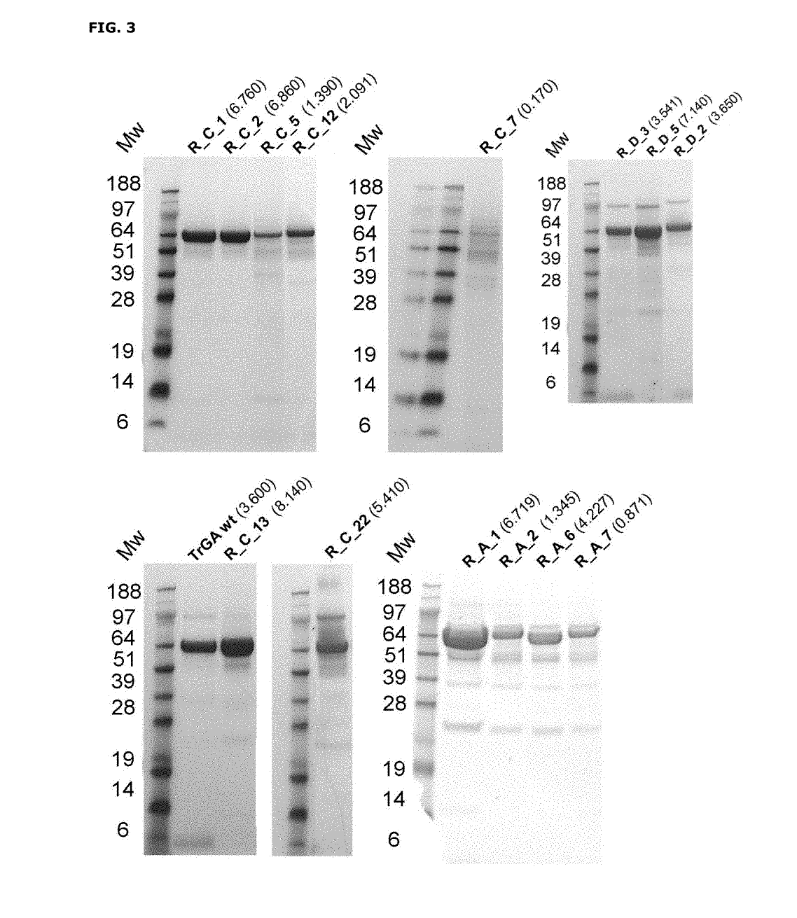

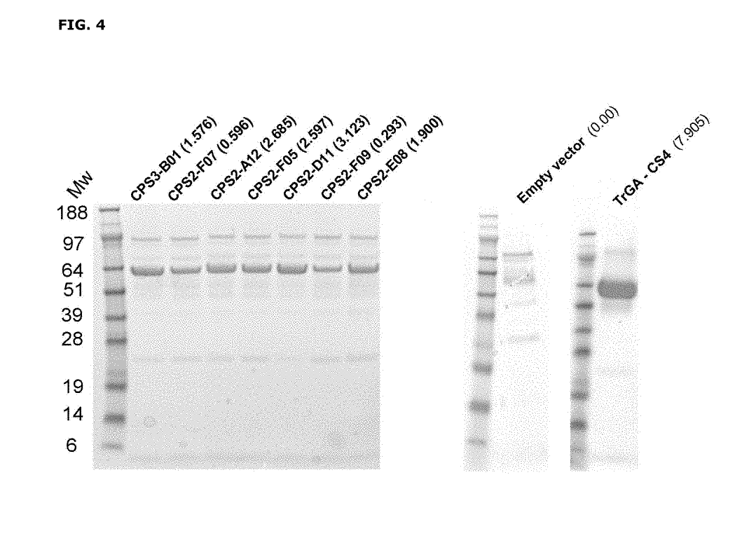

The protein profile of the whole broth samples was determined by sodium dodecyl sulfate polyacrylamide gel electrophoresis (SDS-PAGE electrophoresis). Samples of the culture supernatant were mixed with an equal volume of 5× sample loading buffer with reducing agent, boiled for 10 min and separated on NUPAGE® Novex 4-12% Bis-Tris Gel with MES SDS Running Buffer (Invitrogen, Carlsbad, Calif., USA). Polypeptide bands were visualized in the SDS gel with SIMPLYBLUE SafeStain (Invitrogen, Carlsbad, Calif., USA) according to the manufacturer's protocol. As depicted in FIGS. 3, 4 and 5, the fermentation broth of Trichoderma reesei variants from both the TrGA (wt) and TrGA-CS4 backbone (CS4 including L417V-T430A-Q511H-A539R-N563I compared to TrGA (wt)) were analyzed. Glucoamylase activity of the fermentation broth of Trichoderma reesei variants was measured and is shown in table 2.

TABLE 2
Glucoamylase activity of the fermentation broth of Trichorderma reesei
glucoamylase variants. Activity (GAU/ml) was measured with a
pNP-β-maltoside assay and results are an average of three measurements.

| [GAU/ml] | Act. | Std | Act. | Std | Act. | Std | Act. | Std | Act. | Std |
|---|---|---|---|---|---|---|---|---|---|---|
| | CPS3- | | CPS2- | | CPS2- | | CPS2- | | TrGA | |
| | B01 | | F07 | | A12 | | F05 | | (CS4) | |
| Activity | 1.576 | 0.11 | 0.596 | 0.09 | 2.685 | 0.21 | 2.597 | 0.14 | 7.905 | 0.17 |
| | CPS2- | | CPS2- | | CPS2- | | R_A_1 | | R_A_2 | |
| | D11 | | F09 | | E08 | | | | | |
| Activity | 3.213 | 0.09 | 0.293 | 0.01 | 1.900 | 0.12 | 6.72 | 0.21 | 1.35 | 0.13 |
| | R_A_6 | | R_A_7 | | R_C_1 | | R_C_2 | | R_C_5 | |
| Activity | 4.23 | 0.35 | 0.87 | 0.10 | 6.76 | 0.15 | 6.86 | 0.05 | 1.390 | 0.08 |
| | | | | | | | | | TrGA | |
| | R_C_7 | | R_C_12 | | R_C_13 | | R_C_22 | | (wt) | |
| Activity | 0.170 | 0.02 | 2.091 | 0.17 | 8.140 | 0.12 | 5.410 | 0.12 | 3.600 | 0.11 |
| | R_D_2 | | R_D_2 | | R_D_5 | | | | | |
| Activity | 3.65 | 0.12 | 3.541 | 0.19 | 7.140 | | | | | |

The fermentation broth of most Trichoderma reesei variants showed an intense protein band at the size of the TrGA (wt) glucoamylase (64 kDa). However large variation was seen in expression levels of the different variants and also in the measured GAU activity between the fermented broths. Comparing the total GAU activity of the ferment broth with the total protein content, the apperant specific activity was seen to vary 52-fold from the variants with the highest specific activity to one with the lowest. Thus several combinations of mutations involving certain sites were either destructive in terms expressibility or GAU activity, and left out of brew analysis.

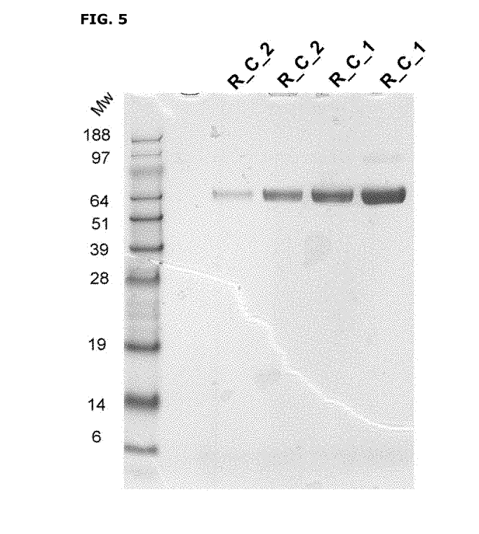

SDS-PAGE analysis of the purified R_C_1 and R_C_2 variants from the one step β-cyclodextrin chromatography is shown in FIG. 5. All purified variants were desalted on a PD-10 column (GE Healthcare, cat no. 17-0851-01) equillibrated in 25 mM Na-acetat pH 4.3 (Sigma, 28705) to avoid any inhibition of remaining a-cyclodextrin.

### Example 4: Thermo Stability Assay of Glucoamylase Variants from Trichoderma reesei (TrGA)

The thermal stability was measured according to above assay “Thermal stability assay”.

The results of the Thermo stability assay are shown in Table 3 with the residual activity for the variants, which were selected from an initial screen for expression and fermentation in large scale: CPS3-B01, CPS2-F07, CPS2-A12, CPS2-F05, CPS2-D11, CPS2-F09, CPS2-E08, R_A_1, R_A_2, R_A_6, R_A_7, R_C_1, R_C_2, R_C_5, R_C_7, R_C_12, R_C_13, R_C_22, R_D_2, R_D_3 and R_D_5. The parent molecule (TrGA-wt and TrGA-CS4) under the conditions described showed a residual activity of 24 and 38% respectively after pasteurization for 100 seconds. A glucoamylase from Aspergillus niger, DIAZYME® X4, was included for benchmark and showed a residual activity of 45% after 100 seconds of incubation. The material used was purified and desalted protein (25 mM Na-acetat pH 4.3). Residual activity was calculated on basis of GAU activity (pNP-β-maltoside substrate) before and after increasing (up to 100 sec) incubation in regular degassed Pilsner (Royal Export Pilsner) pH (4.5) at 72° C. Residual activity is shown as a function of incubation time: 0, 10, 20, 30, 40, 50, 70 and 100 sec and corresponding pasteurizations units: 0.0, 0.0, 0.0, 0.2, 1.6, 4.0, 16.8 and 42.6 PU. Selection of relevant variants for the FV application was defined as the set of variants completely inactivated by 16.8 PU. This leave the following 14 variants of interest: CPS3-B01, CPS2-F07, CPS2-A12, CPS2-F05, CPS2-D11, R_A_1, R_A_6, R_C_1, R_C_2, R_C_5, R_C_7, R_C_13, and R_C_22.

Different minimum PU may be used depending on beer type, raw materials and microbial contamination, brewer and perceived effect on beer flavour. Typically, for beer pasteurisation, 14-16 PU are required. Depending on the pasteurising equipment, pasteurisation temperatures are typically in the range of 64-72 degrees Celsius with a pasteurisation time calculated accordingly. Further information may be found in “Technology Brewing and Malting” by Wolfgang Kunze of the Research and Teaching Institute of Brewing, Berlin (VLB), 3rd completely updated edition, 2004, ISBN 3-921690-49-8.

In comparison thermostability was determined for MkGA I, MkGA II, DIAZYME® X4 (AnGA), TrGA (wt) and TrGA-CS4 in regular degassed Pilsner (Royal Export Pilsner) pH (4.5) as described above. MkGA I, a truncated glucoamylase lacking the SDB was completely inactivated with less than 26 pasteurisation units (PU) using a pasteurisation temperature of 72° C. (previously described in EP 12151285.9), MkGA II required 100 PU and AnGA and TrGA needed more than 200PU to be inactivated.

TABLE 3
Residual glucoamylase activity measured after pasteurisation at 72° C. in regular
pilsner beer at various time/PU. Results are an average of three measurements.

| | | | | | | | | | | CPS2- | |
|---|---|---|---|---|---|---|---|---|---|---|---|
| Time | | | | CPS3- | | CPS2- | | CPS2- | | F05 | |
| [sec] | | Diazyme ® | Res. | B01 | Res. | F07 | Res. | A12 | | Res. | |
| PU | | X4 | Act. | Std | Act. | Std | Act. | Std | Std | Act. | Std |
| 0 | 0.0 | 1.00 | 0.05 | 1.00 | 0.07 | 1.00 | 0.17 | 1.00 | 0.15 | 1.00 | 0.15 |
| 10 | 0.0 | 0.96 | 0.07 | 1.00 | 0.10 | 0.98 | 0.10 | 1.00 | 0.09 | 0.94 | 0.09 |
| 20 | 0.0 | 0.91 | 0.07 | 0.98 | 0.06 | 0.39 | 0.06 | 0.94 | 0.06 | 0.85 | 0.06 |
| 30 | 0.2 | 0.83 | 0.08 | 0.12 | 0.02 | 0.04 | 0.01 | 0.21 | 0.02 | 0.81 | 0.02 |
| 40 | 1.6 | 0.76 | 0.09 | 0.01 | 0.001 | 0.01 | 0.001 | 0.01 | 0.002 | 0.49 | 0.01 |
| 50 | 4.0 | 0.67 | 0.07 | 0.01 | 0.001 | 0.00 | 0.000 | 0.01 | 0.001 | 0.21 | 0.002 |
| 70 | 16.8 | 0.62 | 0.05 | 0.00 | 0.000 | 0.00 | 0.000 | 0.00 | 0.000 | 0.00 | 0.001 |
| 100 | 42.6 | 0.47 | 0.02 | 0.00 | 0.000 | 0.00 | 0.000 | 0.00 | 0.000 | 0.00 | 0.000 |
| | | | | | | CPS2- | | | | | |
| Time | | CPS2- | | CPS2- | | E08 | | R_A_1 | | R_A_2 | |
| [sec] | | D11 | | F09 | | Res. | | Res. | | Res. | |
| PU | | Res. Act. | Std | Res. Act. | Std | Act. | Std | Act. | Std | Act. | Std |
| 0 | 0.0 | 1.00 | 0.13 | 1.00 | 0.05 | 1.00 | 0.09 | 1.00 | 0.09 | 1.00 | 0.11 |
| 10 | 0.0 | 0.97 | 0.10 | 0.92 | 0.12 | 0.99 | 0.10 | 0.89 | 0.12 | 0.90 | 0.06 |
| 20 | 0.0 | 0.96 | 0.06 | 0.43 | 0.05 | 0.96 | 0.08 | 0.79 | 0.07 | 0.81 | 0.05 |
| 30 | 0.2 | 0.11 | 0.02 | 0.05 | 0.06 | 0.16 | 0.03 | 0.41 | 0.04 | 0.57 | 0.07 |
| 40 | 1.6 | 0.01 | 0.001 | 0.04 | 0.005 | 0.02 | 0.005 | 0.02 | 0.002 | 0.10 | 0.005 |
| 50 | 4.0 | 0.01 | 0.001 | 0.04 | 0.003 | 0.01 | 0.002 | 0.00 | 0.000 | 0.03 | 0.003 |
| 70 | 16.8 | 0.00 | 0.000 | 0.04 | 0.008 | 0.01 | 0.002 | 0.00 | 0.000 | 0.02 | 0.001 |
| 100 | 42.6 | 0.00 | 0.000 | 0.01 | 0.001 | 0.01 | 0.001 | 0.00 | 0.000 | 0.01 | 0.001 |
| Time | | R_A_6 | | R_A_7 | | R_C_1 | | R_C_2 | | R_C_5 | |
| [sec] | | Res. | | Res. | | Res. | | Res. | | Res. | |
| PU | | Act. | Std | Act. | Std | Act. | Std | Act. | Std | Act. | Std |
| 0 | 0.0 | 1.00 | 0.15 | 1.00 | 0.09 | 1.00 | 0.10 | 1.00 | 0.06 | 1.00 | 0.07 |
| 10 | 0.0 | 0.83 | 0.09 | 0.82 | 0.08 | 0.80 | 0.07 | 0.70 | 0.05 | 0.38 | 0.03 |
| 20 | 0.0 | 0.62 | 0.06 | 0.68 | 0.06 | 0.20 | 0.02 | 0.02 | 0.02 | 0.05 | 0.001 |
| 30 | 0.2 | 0.25 | 0.02 | 0.36 | 0.02 | 0.01 | 0.001 | 0.01 | 0.001 | 0.02 | 0.001 |
| 40 | 1.6 | 0.00 | 0.000 | 0.05 | 0.002 | 0.00 | 0.001 | 0.00 | 0.001 | 0.02 | 0.001 |
| 50 | 4.0 | 0.00 | 0.000 | 0.04 | 0.001 | 0.00 | 0.001 | 0.00 | 0.001 | 0.00 | 0.000 |
| 70 | 16.8 | 0.00 | 0.000 | 0.02 | 0.001 | 0.00 | 0.001 | 0.00 | 0.000 | 0.00 | 0.000 |
| 100 | 42.6 | 0.00 | 0.000 | 0.00 | 0.000 | 0.00 | 0.001 | 0.00 | 0.000 | 0.00 | 0.000 |
| | | | | | | | | | | TrGA | |
| Time | | R_C_7 | | R_C_12 | | R_C_13 | | R_C_22 | | (wt) | |
| [sec] | | Res. | | Res. | | Res. | | Res. | | Res. | |
| PU | | Act. | Std | Act. | Std | Act. | Std | Act. | Std | Act. | Std |
| 0 | 0.0 | 1.00 | 0.11 | 1.00 | 0.09 | 1.00 | 0.13 | 1.00 | 0.10 | 1.00 | 0.06 |
| 10 | 0.0 | 0.67 | 0.08 | 0.35 | 0.02 | 0.99 | 0.10 | 0.79 | 0.10 | 0.96 | 0.04 |
| 20 | 0.0 | 0.08 | 0.01 | 0.03 | 0.02 | 0.28 | 0.04 | 0.30 | 0.01 | 0.94 | 0.07 |
| 30 | 0.2 | 0.04 | 0.01 | 0.03 | 0.001 | 0.01 | 0.001 | 0.01 | 0.01 | 0.90 | 0.05 |
| 40 | 1.6 | 0.02 | 0.001 | 0.02 | 0.001 | 0.01 | 0.001 | 0.00 | 0.001 | 0.61 | 0.07 |
| 50 | 4.0 | 0.00 | 0.001 | 0.02 | 0.002 | 0.01 | 0.000 | 0.00 | 0.001 | 0.47 | 0.06 |
| 70 | 16.8 | 0.00 | 0.000 | 0.02 | 0.001 | 0.00 | 0.000 | 0.00 | 0.001 | 0.29 | 0.03 |
| 100 | 42.6 | 0.00 | 0.000 | 0.01 | 0.001 | 0.00 | 0.000 | 0.00 | 0.000 | 0.24 | 0.02 |
| | | TrGA | | | | | | | | | |

| Time | | (CS4) | | R_D_2 | | R_D_3 | | R_D_5 | |
|---|---|---|---|---|---|---|---|---|---|
| [sec] | | Res. | | Res. | | Res. | | Res. | |
| PU | | Act. | Std | Act. | Std | Act. | Std | Act. | Std |
| 0 | 0.0 | 1.00 | 0.06 | 1.00 | 0.09 | 1.00 | 0.05 | 1.00 | 0.07 |
| 10 | 0.0 | 0.99 | 0.08 | 0.98 | 0.09 | 1.00 | 0.09 | 0.87 | 0.07 |
| 20 | 0.0 | 0.92 | 0.06 | 0.90 | 0.06 | 0.99 | 0.09 | 0.80 | 0.08 |
| 30 | 0.2 | 0.87 | 0.05 | 0.67 | 0.08 | 0.80 | 0.05 | 0.70 | 0.04 |
| 40 | 1.6 | 0.85 | 0.07 | 0.27 | 0.05 | 0.26 | 0.02 | 0.11 | 0.01 |
| 50 | 4.0 | 0.79 | 0.06 | 0.10 | 0.003 | 0.04 | 0.002 | 0.03 | 0.001 |
| 70 | 16.8 | 0.49 | 0.02 | 0.02 | 0.001 | 0.02 | 0.001 | 0.02 | 0.001 |
| 100 | 42.6 | 0.38 | 0.02 | 0.01 | 0.002 | 0.02 | 0.001 | 0.02 | 0.001 |

### Example 5: Use of Trichoderma reesei Glucoamylase Variants from Fermentation Broth in the Fermentation Step of Brewing

### Brew Analysis:

The use of M. kaoliang glucoamylase to saccharify wort carbohydrates and support ethanol fermentation was compared to DIAZYME® X4 which comprises a glucoamylase from Aspergillus niger (AnGA), the wild-type glucoamylase from Trichoderma reesei (TrGA wt), the CS4 glucoamylase variant from Trichoderma reesei (TrGA CS4) and two glucoamylases from Monascus kaoling (MkGAI and MkGAII) previously investigated for application in brewing (EP 12151285.9). Fermentation trials were performed using a wort prepared from Munton's malt extract as described in the Assays and Methods section.

Specific gravity of the wort before, during and after fermentation was measured using a specific gravity hydrometer or Anton-Paar density meter (e.g. DMA 4100 M) and Real Attenuation was calculated and expressed in percentage form as RDF according to the formulae listed by Ensminger (see http://hbd.org/ensmingr/ “Beer data: Alcohol, Calorie, and Attenuation Levels of Beer”). The obtained RDF values when enzyme are dosed on mg protein (0.058 mg GA/ml wort) are shown in table 4.

TABLE 4
RDF values determined for the listed GAs (purified proteins)
applied to the FV at similar concentration (0.058 mg GA/ml wort).
Results are an average of two measurements ± std error.

| | No enzymes | CPS3-B01 | CPS2-F07 | CPS2-A12 | CPS2-F05 |
|---|---|---|---|---|---|
| RDF [%] | 60.15 ± 0.16 | 70.58 ± 0.39 | 74.56 ± 0.52 | 74.54 ± 0.06 | 75.19 ± 0.68 |
| | CPS2-D11 | CPS2-F09 | CPS2-E08 | R_A_1 | R_A_2 |
| RDF [%] | 74.63 ± 0.53 | 65.60 ± 0.17 | 68.95 ± 0.11 | 75.28 ± 0.08 | 71.71 ± 0.23 |
| | R_A_6 | R_A_7 | R_C_1 | R_C_2 | R_C_5 |
| RDF [%] | 74.69 ± 0.18 | 73.63 ± 0.09 | 75.15 ± 0.36 | 73.48 ± 0.04 | 69.07 ± 0.20 |
| | R_C_7 | R_C_12 | R_C_13 | R_C_22 | TrGA (wt) |
| RDF [%] | 66.51 ± 0.34 | 72.69 ± 0.11 | 75.76 ± 0.16 | 75.05 ± 0.21 | 75.04 ± 0.13 |
| | TrGA (CS4) | Diazyme ® X4 | MkGAI | MkGAII | |
| RDF [%] | 75.19 ± 0.08 | 74.81 ± 0.37 | 75.57 ± 0.20 | 74.92 ± 0.24 | |

Several variants showed similar performance to the references (TrGA (wt), TrGA (CS4) and Diaxyme® X4) being within standard error, however significant differences were also seen for some of the combinatorial variants. Notably a number of combinatorial variants show markedly decreased performance (decreased RDF %), which may be subscribed to a change in substrate specificity as their performance also were lowered when dosed on GAU activity (CPS3 B01, CPS2 E08, CPS2 F09, R_A_2, R_A_7, R_C_2, R_C_5, R_C_7 and R_C_12). The remaining 9 GA's (CPS2-A12, CPS2-F05, CPS2-D11, CPS2-F07, R_A_1, R_A_6, R_C_1, R_C_13 and R_C_22) produced RDF values comparable/similar to what was obtained by the references (TrGA wt, TrGA CS4 and Diaxyme® X4). None of the tested combinatorial variants significantly increased the RDF value compared to the RDF obtained by the references (TrGA (wt), TrGA-CS4 and Diaxyme®X4) and also the glucoamylases from Monascus kaoliang (MkGAI and MkGAII). Selection of relevant variants for the FV application was defined as the set of variants producing an RDF value of minimum 74.5, when dosed at 0.058 mg GA/ml wort. This leave the following 9 variants of interest: CPS2-A12, CPS2-F05, CPS2-D11, CPS2-F07, R_A_1, R_A_6, R_C_1, R_C_13 and R_C_22.

This set of 9 variants that were functional in the FV, all of them were interesting in terms of thermolability according to the “Thermo stability assay” as described above. Each variant may get completely inactivated by 16.8 PU and produces an RDF value of minimum 74.5, when dosed at 0.058 mg GA/ml wort in the FV under the given set of conditions.

These 9 variants were socalled winner hits in screening both thermolability and saccharification performance.

### SEQUENCES

Following are sequences, which are herein incorporated by reference in their entirety.

SEQ ID NO 1:
Trichoderma reesei glucoamylase, full-length; with signal peptide

| | |
|---|---|
| <210> | 1 |
| <211> | 632 |
| <212> | PRT |
| <213> | Trichoderma reesei |
| <400> | 1 |

```
Met His Val Leu Ser Thr Ala Val Leu Leu Gly Ser Val Ala Val Gln
1 5 10 15
Lys Val Leu Gly Arg Pro Gly Ser Ser Gly Leu Ser Asp Val Thr Lys
                        20 25 30
Arg Ser Val Asp Asp Phe Ile Ser Thr Glu Thr Pro Ile Ala Leu Asn
                35 40 45
Asn Leu Leu Cys Asn Val Gly Pro Asp Gly Cys Arg Ala Phe Gly Thr
        50 55 60
Ser Ala Gly Ala Val Ile Ala Ser Pro Ser Thr Ile Asp Pro Asp Tyr
65 70 75 80
Tyr Tyr Met Trp Thr Arg Asp Ser Ala Leu Val Phe Lys Asn Leu Ile
                                85 90 95
Asp Arg Phe Thr Glu Thr Tyr Asp Ala Gly Leu Gln Arg Arg Ile Glu
                        100 105 110
Gln Tyr Ile Thr Ala Gln Val Thr Leu Gln Gly Leu Ser Asn Pro Ser
                115 120 125
Gly Ser Leu Ala Asp Gly Ser Gly Leu Gly Glu Pro Lys Phe Glu Leu
        130 135 140
Thr Leu Lys Pro Phe Thr Gly Asn Trp Gly Arg Pro Gln Arg Asp Gly
145 150 155 160
Pro Ala Leu Arg Ala Ile Ala Leu Ile Gly Tyr Ser Lys Trp Leu Ile
                                165 170 175
Asn Asn Asn Tyr Gln Ser Thr Val Ser Asn Val Ile Trp Pro Ile Val
                        180 185 190
Arg Asn Asp Leu Asn Tyr Val Ala Gln Tyr Trp Asn Gln Thr Gly Phe
                195 200 205
Asp Leu Trp Glu Glu Val Asn Gly Ser Ser Phe Phe Thr Val Ala Asn
        210 215 220
Gln His Arg Ala Leu Val Glu Gly Ala Thr Leu Ala Ala Thr Leu Gly
225 230 235 240
Gln Ser Gly Ser Ala Tyr Ser Ser Val Ala Pro Gln Val Leu Cys Phe
                                245 250 255
Leu Gln Arg Phe Trp Val Ser Ser Gly Gly Tyr Val Asp Ser Asn Ile
                        260 265 270
Asn Thr Asn Glu Gly Arg Thr Gly Lys Asp Val Asn Ser Val Leu Thr
                275 280 285
Ser Ile His Thr Phe Asp Pro Asn Leu Gly Cys Asp Ala Gly Thr Phe
        290 295 300
Gln Pro Cys Ser Asp Lys Ala Leu Ser Asn Leu Lys Val Val Val Asp
305 310 315 320
Ser Phe Arg Ser Ile Tyr Gly Val Asn Lys Gly Ile Pro Ala Gly Ala
                                325 330 335
Ala Val Ala Ile Gly Arg Tyr Ala Glu Asp Val Tyr Tyr Asn Gly Asn
                        340 345 350
Pro Trp Tyr Leu Ala Thr Phe Ala Ala Ala Glu Gln Leu Tyr Asp Ala
                355 360 365
Ile Tyr Val Trp Lys Lys Thr Gly Ser Ile Thr Val Thr Ala Thr Ser
        370 375 380
Leu Ala Phe Phe Gln Glu Leu Val Pro Gly Val Thr Ala Gly Thr Tyr
385 390 395 400
Ser Ser Ser Ser Ser Thr Phe Thr Asn Ile Ile Asn Ala Val Ser Thr
                                405 410 415
Tyr Ala Asp Gly Phe Leu Ser Glu Ala Ala Lys Tyr Val Pro Ala Asp
                        420 425 430
Gly Ser Leu Ala Glu Gln Phe Asp Arg Asn Ser Gly Thr Pro Leu Ser
                435 440 445
Ala Leu His Leu Thr Trp Ser Tyr Ala Ser Phe Leu Thr Ala Thr Ala
        450 455 460
Arg Arg Ala Gly Ile Val Pro Pro Ser Trp Ala Asn Ser Ser Ala Ser
465 470 475 480
Thr Ile Pro Ser Thr Cys Ser Gly Ala Ser Val Val Gly Ser Tyr Ser
                                485 490 495
Arg Pro Thr Ala Thr Ser Phe Pro Pro Ser Gln Thr Pro Lys Pro Gly
                        500 505 510
Val Pro Ser Gly Thr Pro Tyr Thr Pro Leu Pro Cys Ala Thr Pro Thr
                515 520 525
Ser Val Ala Val Thr Phe His Glu Leu Val Ser Thr Gln Phe Gly Gln
        530 535 540
Thr Val Lys Val Ala Gly Asn Ala Ala Ala Leu Gly Asn Trp Ser Thr
545 550 555 560
Ser Ala Ala Val Ala Leu Asp Ala Val Asn Tyr Ala Asp Asn His Pro
                                565 570 575
Leu Trp Ile Gly Thr Val Asn Leu Glu Ala Gly Asp Val Val Glu Tyr
                        580 585 590
Lys Tyr Ile Asn Val Gly Gln Asp Gly Ser Val Thr Trp Glu Ser Asp
                595 600 605
Pro Asn His Thr Tyr Thr Val Pro Ala Val Ala Cys Val Thr Gln Val
        610 615 620
Val Lys Glu Asp Thr Trp Gln Ser
625 630
SEQ ID NO: 2:
Trichoderma reesei glucoamylase, mature protein; without signal
peptide
```

| | |
|---|---|
| <210> | 2 |
| <211> | 599 |
| <212> | PRT |
| <213> | Trichoderma reesei |
| <400> | 2 |

```
Ser Val Asp Asp Phe Ile Ser Thr Glu Thr Pro Ile Ala Leu Asn Asn
1 5 10 15
Leu Leu Cys Asn Val Gly Pro Asp Gly Cys Arg Ala Phe Gly Thr Ser
                        20 25 30
Ala Gly Ala Val Ile Ala Ser Pro Ser Thr Ile Asp Pro Asp Tyr Tyr
                35 40 45
Tyr Met Trp Thr Arg Asp Ser Ala Leu Val Phe Lys Asn Leu Ile Asp
        50 55 60
Arg Phe Thr Glu Thr Tyr Asp Ala Gly Leu Gln Arg Arg Ile Glu Gln
65 70 75 80
Tyr Ile Thr Ala Gln Val Thr Leu Gln Gly Leu Ser Asn Pro Ser Gly
                                85 90 95
Ser Leu Ala Asp Gly Ser Gly Leu Gly Glu Pro Lys Phe Glu Leu Thr
                        100 105 110
Leu Lys Pro Phe Thr Gly Asn Trp Gly Arg Pro Gln Arg Asp Gly Pro
                115 120 125
Ala Leu Arg Ala Ile Ala Leu Ile Gly Tyr Ser Lys Trp Leu Ile Asn
        130 135 140
Asn Asn Tyr Gln Ser Thr Val Ser Asn Val Ile Trp Pro Ile Val Arg
145 150 155 160
Asn Asp Leu Asn Tyr Val Ala Gln Tyr Trp Asn Gln Thr Gly Phe Asp
                                165 170 175
Leu Trp Glu Glu Val Asn Gly Ser Ser Phe Phe Thr Val Ala Asn Gln
                        180 185 190
His Arg Ala Leu Val Glu Gly Ala Thr Leu Ala Ala Thr Leu Gly Gln
                195 200 205
Ser Gly Ser Ala Tyr Ser Ser Val Ala Pro Gln Val Leu Cys Phe Leu
        210 215 220
Gln Arg Phe Trp Val Ser Ser Gly Gly Tyr Val Asp Ser Asn Ile Asn
225 230 235 240
Thr Asn Glu Gly Arg Thr Gly Lys Asp Val Asn Ser Val Leu Thr Ser
                                245 250 255
Ile His Thr Phe Asp Pro Asn Leu Gly Cys Asp Ala Gly Thr Phe Gln
                        260 265 270
Pro Cys Ser Asp Lys Ala Leu Ser Asn Leu Lys Val Val Val Asp Ser
                275 280 285
Phe Arg Ser Ile Tyr Gly Val Asn Lys Gly Ile Pro Ala Gly Ala Ala
        290 295 300
Val Ala Ile Gly Arg Tyr Ala Glu Asp Val Tyr Tyr Asn Gly Asn Pro
305 310 315 320
Trp Tyr Leu Ala Thr Phe Ala Ala Ala Glu Gln Leu Tyr Asp Ala Ile
                                325 330 335
Tyr Val Trp Lys Lys Thr Gly Ser Ile Thr Val Thr Ala Thr Ser Leu
                        340 345 350
Ala Phe Phe Gln Glu Leu Val Pro Gly Val Thr Ala Gly Thr Tyr Ser
                355 360 365
Ser Ser Ser Ser Thr Phe Thr Asn Ile Ile Asn Ala Val Ser Thr Tyr
        370 375 380
Ala Asp Gly Phe Leu Ser Glu Ala Ala Lys Tyr Val Pro Ala Asp Gly
385 390 395 400
Ser Leu Ala Glu Gln Phe Asp Arg Asn Ser Gly Thr Pro Leu Ser Ala
                                405 410 415
Leu His Leu Thr Trp Ser Tyr Ala Ser Phe Leu Thr Ala Thr Ala Arg
                        420 425 430
Arg Ala Gly Ile Val Pro Pro Ser Trp Ala Asn Ser Ser Ala Ser Thr
                435 440 445
Ile Pro Ser Thr Cys Ser Gly Ala Ser Val Val Gly Ser Tyr Ser Arg
        450 455 460
Pro Thr Ala Thr Ser Phe Pro Pro Ser Gln Thr Pro Lys Pro Gly Val
465 470 475 480
Pro Ser Gly Thr Pro Tyr Thr Pro Leu Pro Cys Ala Thr Pro Thr Ser
                                485 490 495
Val Ala Val Thr Phe His Glu Leu Val Ser Thr Gln Phe Gly Gln Thr
                        500 505 510
Val Lys Val Ala Gly Asn Ala Ala Ala Leu Gly Asn Trp Ser Thr Ser
                515 520 525
Ala Ala Val Ala Leu Asp Ala Val Asn Tyr Ala Asp Asn His Pro Leu
        530 535 540
Trp Ile Gly Thr Val Asn Leu Glu Ala Gly Asp Val Val Glu Tyr Lys
545 550 555 560
Tyr Ile Asn Val Gly Gln Asp Gly Ser Val Thr Trp Glu Ser Asp Pro
                                565 570 575
Asn His Thr Tyr Thr Val Pro Ala Val Ala Cys Val Thr Gln Val Val
                        580 585 590
Lys Glu Asp Thr Trp Gln Ser
                595
SEQ ID NO: 3:
Trichoderma reesei glucoamylase catalytic domain, 1-453 of mature TrGA,
CD
```

| | |
|---|---|
| <210> | 3 |
| <211> | 453 |
| <212> | PRT |
| <213> | Trichoderma reesei |
| <400> | 3 |

```
Ser Val Asp Asp Phe Ile Ser Thr Glu Thr Pro Ile Ala Leu Asn Asn
1 5 10 15
Leu Leu Cys Asn Val Gly Pro Asp Gly Cys Arg Ala Phe Gly Thr Ser
                        20 25 30
Ala Gly Ala Val Ile Ala Ser Pro Ser Thr Ile Asp Pro Asp Tyr Tyr
                35 40 45
Tyr Met Trp Thr Arg Asp Ser Ala Leu Val Phe Lys Asn Leu Ile Asp
        50 55 60
Arg Phe Thr Glu Thr Tyr Asp Ala Gly Leu Gln Arg Arg Ile Glu Gln
65 70 75 80
Tyr Ile Thr Ala Gln Val Thr Leu Gln Gly Leu Ser Asn Pro Ser Gly
                                85 90 95
Ser Leu Ala Asp Gly Ser Gly Leu Gly Glu Pro Lys Phe Glu Leu Thr
                        100 105 110
Leu Lys Pro Phe Thr Gly Asn Trp Gly Arg Pro Gln Arg Asp Gly Pro
                115 120 125
Ala Leu Arg Ala Ile Ala Leu Ile Gly Tyr Ser Lys Trp Leu Ile Asn
        130 135 140
Asn Asn Tyr Gln Ser Thr Val Ser Asn Val Ile Trp Pro Ile Val Arg
145 150 155 160
Asn Asp Leu Asn Tyr Val Ala Gln Tyr Trp Asn Gln Thr Gly Phe Asp
                                165 170 175
Leu Trp Glu Glu Val Asn Gly Ser Ser Phe Phe Thr Val Ala Asn Gln
                        180 185 190
His Arg Ala Leu Val Glu Gly Ala Thr Leu Ala Ala Thr Leu Gly Gln
                195 200 205
Ser Gly Ser Ala Tyr Ser Ser Val Ala Pro Gln Val Leu Cys Phe Leu
        210 215 220
Gln Arg Phe Trp Val Ser Ser Gly Gly Tyr Val Asp Ser Asn Ile Asn
225 230 235 240
Thr Asn Glu Gly Arg Thr Gly Lys Asp Val Asn Ser Val Leu Thr Ser
                                245 250 255
Ile His Thr Phe Asp Pro Asn Leu Gly Cys Asp Ala Gly Thr Phe Gln
                        260 265 270
Pro Cys Ser Asp Lys Ala Leu Ser Asn Leu Lys Val Val Val Asp Ser
                275 280 285
Phe Arg Ser Ile Tyr Gly Val Asn Lys Gly Ile Pro Ala Gly Ala Ala
        290 295 300
Val Ala Ile Gly Arg Tyr Ala Glu Asp Val Tyr Tyr Asn Gly Asn Pro
305 310 315 320
Trp Tyr Leu Ala Thr Phe Ala Ala Ala Glu Gln Leu Tyr Asp Ala Ile
                                325 330 335
Tyr Val Trp Lys Lys Thr Gly Ser Ile Thr Val Thr Ala Thr Ser Leu
                        340 345 350
Ala Phe Phe Gln Glu Leu Val Pro Gly Val Thr Ala Gly Thr Tyr Ser
                355 360 365
Ser Ser Ser Ser Thr Phe Thr Asn Ile Ile Asn Ala Val Ser Thr Tyr
        370 375 380
Ala Asp Gly Phe Leu Ser Glu Ala Ala Lys Tyr Val Pro Ala Asp Gly
385 390 395 400
Ser Leu Ala Glu Gln Phe Asp Arg Asn Ser Gly Thr Pro Leu Ser Ala
                                405 410 415
Leu His Leu Thr Trp Ser Tyr Ala Ser Phe Leu Thr Ala Thr Ala Arg
                        420 425 430
Arg Ala Gly Ile Val Pro Pro Ser Trp Ala Asn Ser Ser Ala Ser Thr
                435 440 445
Ile Pro Ser Thr Cys
        450
SEQ ID NO: 4:
Trichoderma reesei glucoamylase cDNA
```

| | |
|---|---|
| <210> | 4 |
| <211> | 1899 |
| <212> | DNA |
| <213> | Trichoderma reesei |
| <400> | 4 |

| | |
|---|---|
| atgcacgtcc tgtcgactgc ggtgctgctc ggctccgttg ccgttcaaaa ggtcctggga | 60 |
| agaccaggat caagcggtct gtccgacgtc accaagaggt ctgttgacga cttcatcagc | 120 |
| accgagacgc ctattgcact gaacaatctt ctttgcaatg ttggtcctga tggatgccgt | 180 |
| gcattcggca catcagctgg tgcggtgatt gcatctccca gcacaattga cccggactac | 240 |
| tattacatgt ggacgcgaga tagcgctctt gtcttcaaga acctcatcga ccgcttcacc | 300 |
| gaaacgtacg atgcgggcct gcagcgccgc atcgagcagt acattactgc ccaggtcact | 360 |
| ctccagggcc tctctaaccc ctcgggctcc ctcgcggacg gctctggtct cggcgagccc | 420 |
| aagtttgagt tgaccctgaa gcctttcacc ggcaactggg gtcgaccgca gcgggatggc | 480 |
| ccagctctgc gagccattgc cttgattgga tactcaaagt ggctcatcaa caacaactat | 540 |
| cagtcgactg tgtccaacgt catctggcct attgtgcgca acgacctcaa ctatgttgcc | 600 |
| cagtactgga accaaaccgg ctttgacctc tgggaagaag tcaatgggag ctcattcttt | 660 |
| actgttgcca accagcaccg agcacttgtc gagggcgcca ctcttgctgc cactcttggc | 720 |
| cagtcgggaa gcgcttattc atctgttgct ccccaggttt tgtgctttct ccaacgattc | 780 |
| tgggtgtcgt ctggtggata cgtcgactcc aacatcaaca ccaacgaggg caggactggc | 840 |
| aaggatgtca actccgtcct gacttccatc cacaccttcg atcccaacct tggctgtgac | 900 |
| gcaggcacct tccagccatg cagtgacaaa gcgctctcca acctcaaggt tgttgtcgac | 960 |
| tccttccgct ccatctacgg cgtgaacaag ggcattcctg ccggtgctgc cgtcgccatt | 1020 |
| ggccggtatg cagaggatgt gtactacaac ggcaaccctt ggtatcttgc tacatttgct | 1080 |
| gctgccgagc agctgtacga tgccatctac gtctggaaga agacgggctc catcacggtg | 1140 |
| accgccacct ccctggcctt cttccaggag cttgttcctg gcgtgacggc cgggacctac | 1200 |
| tccagcagct cttcgacctt taccaacatc atcaacgccg tctcgacata cgccgatggc | 1260 |
| ttcctcagcg aggctgccaa gtacgtcccc gccgacggtt cgctggccga gcagtttgac | 1320 |
| cgcaacagcg gcactccgct gtctgcgctt cacctgacgt ggtcgtacgc ctcgttcttg | 1380 |
| acagccacgg cccgtcgggc tggcatcgtg cccccctcgt gggccaacag cagcgctagc | 1440 |
| acgatcccct cgacgtgctc cggcgcgtcc gtggtcggat cctactcgcg tcccaccgcc | 1500 |
| acgtcattcc ctccgtcgca gacgcccaag cctggcgtgc cttccggtac tccctacacg | 1560 |
| cccctgccct gcgcgacccc aacctccgtg gccgtcacct tccacgagct cgtgtcgaca | 1620 |
| cagtttggcc agacggtcaa ggtggcgggc aacgccgcgg ccctgggcaa ctggagcacg | 1680 |
| agcgccgccg tggctctgga cgccgtcaac tatgccgata accaccccct gtggattggg | 1740 |
| acggtcaacc tcgaggctgg agacgtcgtg gagtacaagt acatcaatgt gggccaagat | 1800 |
| ggctccgtga cctgggagag tgatcccaac cacacttaca cggttcctgc ggtggcttgt | 1860 |
| gtgacgcagg ttgtcaagga ggacacctgg cagtcgtaa | 1899 |

SEQ ID NO: 5:
Aspergillus awamori GA (AaGA); CD

| | |
|---|---|
| <210> | 5 |
| <211> | 448 |
| <212> | PRT |
| <213> | Aspergillus awamori |
| <400> | 5 |

```
Ala Thr Leu Asp Ser Trp Leu Ser Asn Glu Ala Thr Val Ala Arg Thr
1 5 10 15
Ala Ile Leu Asn Asn Ile Gly Ala Asp Gly Ala Trp Val Ser Gly Ala
                        20 25 30
Asp Ser Gly Ile Val Val Ala Ser Pro Ser Thr Asp Asn Pro Asp Tyr
                35 40 45
Phe Tyr Thr Trp Thr Arg Asp Ser Gly Leu Val Ile Lys Thr Leu Val
        50 55 60
Asp Leu Phe Arg Asn Gly Asp Thr Asp Leu Leu Ser Thr Ile Glu Asn
65 70 75 80
Tyr Ile Ser Ser Gln Ala Ile Val Gln Gly Ile Ser Asn Pro Ser Gly
                                85 90 95
Asp Leu Ser Ser Gly Gly Leu Gly Glu Pro Lys Phe Asn Val Asp Glu
                        100 105 110
Thr Ala Tyr Thr Gly Ser Trp Gly Arg Pro Gln Arg Asp Gly Pro Ala
                115 120 125
Leu Arg Ala Thr Ala Met Ile Gly Phe Arg Gln Trp Leu Leu Asp Asn
        130 135 140
Gly Tyr Thr Ser Ala Ala Thr Glu Ile Val Trp Pro Leu Val Arg Asn
145 150 155 160
Asp Leu Ser Tyr Val Ala Gln Tyr Trp Asn Gln Thr Gly Tyr Asp Leu
                                165 170 175
Trp Glu Glu Val Asn Gly Ser Ser Phe Phe Thr Ile Ala Val Gln His
                        180 185 190
Arg Ala Leu Val Glu Gly Ser Ala Phe Ala Thr Ala Val Gly Ser Ser
                195 200 205
Cys Ser Trp Cys Asp Ser Gln Ala Pro Gln Ile Leu Cys Tyr Leu Gln
        210 215 220
Ser Phe Trp Thr Gly Glu Tyr Ile Leu Ala Asn Phe Asp Ser Ser Arg
225 230 235 240
Ser Gly Lys Asp Thr Asn Thr Leu Leu Gly Ser Ile His Thr Phe Asp
                                245 250 255
Pro Glu Ala Gly Cys Asp Asp Ser Thr Phe Gln Pro Cys Ser Pro Arg
                        260 265 270
Ala Leu Ala Asn His Lys Glu Val Val Asp Ser Phe Arg Ser Ile Tyr
                275 280 285
Thr Leu Asn Asp Gly Leu Ser Asp Ser Glu Ala Val Ala Val Gly Arg
        290 295 300
Tyr Pro Lys Asp Ser Tyr Tyr Asn Gly Asn Pro Trp Phe Leu Cys Thr
305 310 315 320
Leu Ala Ala Ala Glu Gln Leu Tyr Asp Ala Leu Tyr Gln Trp Asp Lys
                                325 330 335
Gln Gly Ser Leu Glu Ile Thr Asp Val Ser Leu Asp Phe Phe Gln Ala
                        340 345 350
Leu Tyr Ser Asp Ala Ala Thr Gly Thr Tyr Ser Ser Ser Ser Ser Thr
                355 360 365
Tyr Ser Ser Ile Val Asp Ala Val Lys Thr Phe Ala Asp Gly Phe Val
        370 375 380
Ser Ile Val Glu Thr His Ala Ala Ser Asn Gly Ser Leu Ser Glu Gln
385 390 395 400
Tyr Asp Lys Ser Asp Gly Asp Glu Leu Ser Ala Arg Asp Leu Thr Trp
                                405 410 415
Ser Tyr Ala Ala Leu Leu Thr Ala Asn Asn Arg Arg Asn Ser Val Met
                        420 425 430
Pro Pro Ser Trp Gly Glu Thr Ser Ala Ser Ser Val Pro Gly Thr Cys
                435 440 445
SEQ ID NO: 6:
Aspergillus niger (AnGA), CD
```

| | |
|---|---|
| <210> | 6 |
| <211> | 449 |
| <212> | PRT |
| <213> | Aspergillus niger |
| <400> | 6 |

```
Ala Thr Leu Asp Ser Trp Leu Ser Asn Glu Ala Thr Val Ala Arg Thr
1 5 10 15
Ala Ile Leu Asn Asn Ile Gly Ala Asp Gly Ala Trp Val Ser Gly Ala
                        20 25 30
Asp Ser Gly Ile Val Val Ala Ser Pro Ser Thr Asp Asn Pro Asp Tyr
                35 40 45
Phe Tyr Thr Trp Thr Arg Asp Ser Gly Leu Val Leu Lys Thr Leu Val
        50 55 60
Asp Leu Phe Arg Asn Gly Asp Thr Ser Leu Leu Ser Thr Ile Glu Asn
65 70 75 80
Tyr Ile Ser Ala Gln Ala Ile Val Gln Gly Ile Ser Asn Pro Ser Gly
                                85 90 95
Asp Leu Ser Ser Gly Ala Gly Leu Gly Glu Pro Lys Phe Asn Val Asp
                        100 105 110
Glu Thr Ala Tyr Thr Gly Ser Trp Gly Arg Pro Gln Arg Asp Gly Pro
                115 120 125
Ala Leu Arg Ala Thr Ala Met Ile Gly Phe Gly Gln Trp Leu Leu Asp
        130 135 140
Asn Gly Tyr Thr Ser Thr Ala Thr Asp Ile Val Trp Pro Leu Val Arg
145 150 155 160
Asn Asp Leu Ser Tyr Val Ala Gln Tyr Trp Asn Gln Thr Gly Tyr Asp
                                165 170 175
Leu Trp Glu Glu Val Asn Gly Ser Ser Phe Phe Thr Ile Ala Val Gln
                        180 185 190
His Arg Ala Leu Val Glu Gly Ser Ala Phe Ala Thr Ala Val Gly Ser
                195 200 205
Ser Cys Ser Trp Cys Asp Ser Gln Ala Pro Glu Ile Leu Cys Tyr Leu
        210 215 220
Gln Ser Phe Trp Thr Gly Ser Phe Ile Leu Ala Asn Phe Asp Ser Ser
225 230 235 240
Arg Ser Gly Lys Asp Ala Asn Thr Leu Leu Gly Ser Ile His Thr Phe
                                245 250 255
Asp Pro Glu Ala Ala Cys Asp Asp Ser Thr Phe Gln Pro Cys Ser Pro
                        260 265 270
Arg Ala Leu Ala Asn His Lys Glu Val Val Asp Ser Phe Arg Ser Ile
                275 280 285
Tyr Thr Leu Asn Asp Gly Leu Ser Asp Ser Glu Ala Val Ala Val Gly
        290 295 300
Arg Tyr Pro Glu Asp Thr Tyr Tyr Asn Gly Asn Pro Trp Phe Leu Cys
305 310 315 320
Thr Leu Ala Ala Ala Glu Gln Leu Tyr Asp Ala Leu Tyr Gln Trp Asp
                                325 330 335
Lys Gln Gly Ser Leu Glu Val Thr Asp Val Ser Leu Asp Phe Phe Lys
                        340 345 350
Ala Leu Tyr Ser Asp Ala Ala Thr Gly Thr Tyr Ser Ser Ser Ser Ser
                355 360 365
Thr Tyr Ser Ser Ile Val Asp Ala Val Lys Thr Phe Ala Asp Gly Phe
        370 375 380
Val Ser Ile Val Glu Thr His Ala Ala Ser Asn Gly Ser Met Ser Glu
385 390 395 400
Gln Tyr Asp Lys Ser Asp Gly Glu Gln Leu Ser Ala Arg Asp Leu Thr
                                405 410 415
Trp Ser Tyr Ala Ala Leu Leu Thr Ala Asn Asn Arg Arg Asn Ser Val
                        420 425 430
Val Pro Ala Ser Trp Gly Glu Thr Ser Ala Ser Ser Val Pro Gly Thr
                435 440 445
Cys
SEQ ID NO: 7:
Aspergillus oryzae (AoGA), CD
```

| | |
|---|---|
| <210> | 7 |
| <211> | 450 |
| <212> | PRT |
| <213> | Aspergillus oryzae |
| <400> | 7 |

```
Gln Ser Asp Leu Asn Ala Phe Ile Glu Ala Gln Thr Pro Ile Ala Lys
1 5 10 15
Gln Gly Tyr Leu Asn Asn Ile Gly Ala Asp Gly Lys Leu Val Glu Gly
                        20 25 30
Ala Ala Ala Gly Ile Val Tyr Ala Ser Pro Ser Lys Ser Asn Pro Asp
                35 40 45
Tyr Phe Tyr Thr Trp Thr Arg Asp Ala Gly Leu Thr Met Glu Glu Tyr
        50 55 60
Ile Glu Gln Phe Ile Gly Gly Asp Ala Thr Leu Glu Ser Thr Ile Gln
65 70 75 80
Asn Tyr Val Asp Ser Gln Ala Asn Glu Gln Ala Val Ser Asn Pro Ser
                                85 90 95
Gly Gly Leu Ser Asp Gly Ser Gly Leu Ala Glu Pro Lys Phe Tyr Tyr
                        100 105 110
Asn Ile Ser Gln Phe Thr Asp Ser Trp Gly Arg Pro Gln Arg Asp Gly
                115 120 125
Pro Ala Leu Arg Ala Ser Ala Leu Ile Ala Tyr Gly Asn Ser Leu Ile
        130 135 140
Ser Ser Asp Lys Gln Ser Val Val Lys Ala Asn Ile Trp Pro Ile Tyr
145 150 155 160
Gln Asn Asp Leu Ser Tyr Val Gly Gln Tyr Trp Asn Gln Thr Gly Phe
                                165 170 175
Asp Leu Trp Glu Glu Val Gln Gly Ser Ser Phe Phe Thr Val Ala Val
                        180 185 190
Gln His Lys Ala Leu Val Glu Gly Asp Ala Phe Ala Lys Ala Leu Gly
                195 200 205
Glu Glu Cys Gln Ala Cys Ser Val Ala Pro Gln Ile Leu Cys His Leu
        210 215 220
Gln Asp Phe Trp Asn Gly Ser Ala Val Leu Ser Asn Leu Pro Thr Asn
225 230 235 240
Gly Arg Ser Gly Leu Asp Thr Asn Ser Leu Leu Gly Ser Ile His Thr
                                245 250 255
Phe Asp Pro Ala Ala Ala Cys Asp Asp Thr Thr Phe Gln Pro Cys Ser
                        260 265 270
Ser Arg Ala Leu Ser Asn His Lys Leu Val Val Asp Ser Phe Arg Ser
                275 280 285
Val Tyr Gly Ile Asn Asn Gly Arg Gly Ala Gly Lys Ala Ala Ala Val
        290 295 300
Gly Pro Tyr Ala Glu Asp Thr Tyr Gln Gly Gly Asn Pro Trp Tyr Leu
305 310 315 320
Thr Thr Leu Val Ala Ala Glu Leu Leu Tyr Asp Ala Leu Tyr Gln Trp
                                325 330 335
Asp Lys Gln Gly Gln Val Asn Val Thr Glu Thr Ser Leu Pro Phe Phe
                        340 345 350
Lys Asp Leu Ser Ser Asn Val Thr Thr Gly Ser Tyr Ala Lys Ser Ser
                355 360 365
Ser Ala Tyr Glu Ser Leu Thr Ser Ala Val Lys Thr Tyr Ala Asp Gly
        370 375 380
Phe Ile Ser Val Val Gln Glu Tyr Thr Pro Asp Gly Gly Ala Leu Ala
385 390 395 400
Glu Gln Tyr Ser Arg Asp Gln Gly Thr Pro Val Ser Ala Ser Asp Leu
                                405 410 415
Thr Trp Ser Tyr Ala Ala Phe Leu Ser Ala Val Gly Arg Arg Asn Gly
                        420 425 430
Thr Val Pro Ala Ser Trp Gly Ser Ser Thr Ala Asn Ala Val Pro Ser
                435 440 445
Gln Cys
        450
SEQ ID NO: 8:
Humicola grisea glucoamylase (HgGA); CD
```

| | |
|---|---|
| <210> | 8 |
| <211> | 441 |
| <212> | PRT |
| <213> | Humicola grisea |
| <400> | 8 |

```
Ala Ala Val Asp Thr Phe Ile Asn Thr Glu Lys Pro Ile Ala Trp Asn
1 5 10 15
Lys Leu Leu Ala Asn Ile Gly Pro Asn Gly Lys Ala Ala Pro Gly Ala
                        20 25 30
Ala Ala Gly Val Val Ile Ala Ser Pro Ser Arg Thr Asp Pro Pro Tyr
                35 40 45
Phe Phe Thr Trp Thr Pro Asp Ala Ala Leu Val Leu Thr Gly Ile Ile
        50 55 60
Glu Ser Leu Gly His Asn Tyr Asn Thr Thr Leu Gln Gln Val Ser Asn
65 70 75 80
Pro Ser Gly Thr Phe Ala Asp Gly Ser Gly Leu Gly Glu Ala Lys Phe
                                85 90 95
Asn Val Asp Leu Thr Ala Phe Thr Gly Glu Trp Gly Arg Pro Gln Arg
                        100 105 110
Asp Gly Pro Pro Leu Arg Ala Ile Ala Leu Ile Gln Tyr Ala Lys Trp
                115 120 125
Leu Ile Ala Asn Gly Tyr Lys Ser Thr Ala Lys Ser Val Val Trp Pro
        130 135 140
Val Val Lys Asn Asp Leu Ala Tyr Thr Ala Gln Tyr Trp Asn Glu Thr
145 150 155 160
Gly Phe Asp Leu Trp Glu Glu Val Pro Gly Ser Ser Phe Phe Thr Ile
                                165 170 175
Ala Ser Ser His Arg Ala Leu Thr Glu Gly Ala Tyr Leu Ala Ala Gln
                        180 185 190
Leu Asp Thr Glu Cys Pro Pro Cys Thr Thr Val Ala Pro Gln Val Leu
                195 200 205
Cys Phe Gln Gln Ala Phe Trp Asn Ser Lys Gly Asn Tyr Val Val Ser
        210 215 220
Thr Ser Thr Ala Gly Glu Tyr Arg Ser Gly Lys Asp Ala Asn Ser Ile
225 230 235 240
Leu Ala Ser Ile His Asn Phe Asp Pro Glu Ala Gly Cys Asp Asn Leu
                                245 250 255
Thr Phe Gln Pro Cys Ser Glu Arg Ala Leu Ala Asn His Lys Ala Tyr
                        260 265 270
Val Asp Ser Phe Arg Asn Leu Tyr Ala Ile Asn Lys Gly Ile Ala Gln
                275 280 285
Gly Lys Ala Val Ala Val Gly Arg Tyr Ser Glu Asp Val Tyr Tyr Asn
        290 295 300
Gly Asn Pro Trp Tyr Leu Ala Asn Phe Ala Ala Ala Glu Gln Leu Tyr
305 310 315 320
Asp Ala Ile Tyr Val Trp Asn Lys Gln Gly Ser Ile Thr Val Thr Ser
                                325 330 335
Val Ser Leu Pro Phe Phe Arg Asp Leu Val Ser Ser Val Ser Thr Gly
                        340 345 350
Thr Tyr Ser Lys Ser Ser Ser Thr Phe Thr Asn Ile Val Asn Ala Val
                355 360 365
Lys Ala Tyr Ala Asp Gly Phe Ile Glu Val Ala Ala Lys Tyr Thr Pro
        370 375 380
Ser Asn Gly Ala Leu Ala Glu Gln Tyr Asp Arg Asn Thr Gly Lys Pro
385 390 395 400
Asp Ser Ala Ala Asp Leu Thr Trp Ser Tyr Ser Ala Phe Leu Ser Ala
                                405 410 415
Ile Asp Arg Arg Ala Gly Leu Val Pro Pro Ser Trp Arg Ala Ser Val
                        420 425 430
Ala Lys Ser Gln Leu Pro Ser Thr Cys
                435 440
SEQ ID NO: 9:
Hypocrea vinosa glucoamylase (HvGA); CD
```

| | |
|---|---|
| <210> | 9 |
| <211> | 452 |
| <212> | PRT |
| <400> | 9 |

```
Ser Val Asp Asp Phe Ile Asn Thr Gln Thr Pro Ile Ala Leu Asn Asn
1 5 10 15
Leu Leu Cys Asn Val Gly Pro Asp Gly Cys Arg Ala Phe Gly Thr Ser
                        20 25 30
Ala Gly Ala Val Ile Ala Ser Pro Ser Thr Thr Asp Pro Asp Tyr Tyr
                35 40 45
Tyr Met Trp Thr Arg Asp Ser Ala Leu Val Phe Lys Asn Ile Val Asp
        50 55 60
Arg Phe Thr Gln Gln Tyr Asp Ala Gly Leu Gln Arg Arg Ile Glu Gln
65 70 75 80
Tyr Ile Ser Ala Gln Val Thr Leu Gln Gly Ile Ser Asn Pro Ser Gly
                                  85 90 95
Ser Leu Ser Asp Gly Ser Gly Leu Gly Glu Pro Lys Phe Glu Leu Thr
                        100 105 110
Leu Ser Gln Phe Thr Gly Asn Trp Gly Arg Pro Gln Arg Asp Gly Pro
                115 120 125
Ala Leu Arg Ala Ile Ala Leu Ile Gly Tyr Ser Lys Trp Leu Ile Asn
        130 135 140
Asn Asn Tyr Gln Ser Thr Val Ser Asn Ile Ile Trp Pro Ile Val Arg
145 150 155 160
Asn Asp Leu Asn Tyr Val Ala Gln Tyr Trp Asn Gln Thr Gly Phe Asp
                                165 170 175
Leu Trp Glu Glu Val Asn Gly Ser Ser Phe Phe Thr Val Ala Asn Gln
                        180 185 190
His Arg Ala Leu Val Glu Gly Ala Thr Leu Ala Ala Thr Leu Gly Gln
                195 200 205
Ser Gly Ser Thr Tyr Ser Ser Val Ala Pro Gln Ile Leu Cys Phe Leu
        210 215 220
Gln Arg Phe Trp Val Ser Gly Gly Tyr Ile Asp Ser Asn Ile Asn Thr
225 230 235 240
Asn Glu Gly Arg Thr Gly Lys Asp Ala Asn Ser Leu Leu Ala Ser Ile
                                245 250 255
His Thr Phe Asp Pro Ser Leu Gly Cys Asp Ala Ser Thr Phe Gln Pro
                        260 265 270
Cys Ser Asp Lys Ala Leu Ser Asn Leu Lys Val Val Val Asp Ser Phe
                275 280 285
Arg Ser Ile Tyr Gly Val Asn Lys Gly Ile Pro Ala Gly Ser Ala Val
        290 295 300
Ala Ile Gly Arg Tyr Pro Glu Asp Val Tyr Phe Asn Gly Asn Pro Trp
305 310 315 320
Tyr Leu Ala Thr Phe Ala Ala Ala Glu Gln Leu Tyr Asp Ser Val Tyr
                                325 330 335
Val Trp Lys Lys Thr Gly Ser Ile Thr Val Thr Ser Thr Ser Ser Ala
                        340 345 350
Phe Phe Gln Glu Leu Val Pro Gly Val Ala Ala Gly Thr Tyr Ser Ser
                355 360 365
Ser Gln Ser Thr Phe Thr Ser Ile Ile Asn Ala Ile Ser Thr Tyr Ala
        370 375 380
Asp Gly Phe Leu Ser Glu Ala Ala Lys Tyr Val Pro Ala Asp Gly Ser
385 390 395 400
Leu Ala Glu Gln Phe Asp Arg Asn Thr Gly Thr Pro Leu Ser Ala Val
                                405 410 415
His Leu Thr Trp Ser Tyr Ala Ser Phe Leu Thr Ala Ala Ala Arg Arg
                        420 425 430
Ala Gly Val Val Pro Pro Ser Trp Ala Ser Ser Gly Ala Asn Thr Val
                435 440 445
Pro Ser Ser Cys
        450
SEQ ID NO: 10:
TrGA, linker region
```

| | |
|---|---|
| <210> | 10 |
| <211> | 37 |
| <212> | PRT |
| <213> | Trichoderma reesei |
| <400> | 10 |

```
Ser Gly Ala Ser Val Val Gly Ser Tyr Ser Arg Pro Thr Ala Thr Ser
1 5 10 15
Phe Pro Pro Ser Gln Thr Pro Lys Pro Gly Val Pro Ser Gly Thr Pro
                                20 25 30
Tyr Thr Pro Leu Pro
                        35
SEQ ID NO: 11:
TrGA, SBD
<210> 11
<211> 109
<212> PRT
<213> Trichoderma reesei
<400> 11
Cys Ala Thr Pro Thr Ser Val Ala Val Thr Phe His Glu Leu Val Ser
1 5 10 15
Thr Gln Phe Gly Gln Thr Val Lys Val Ala Gly Asn Ala Ala Ala Leu
                        20 25 30
Gly Asn Trp Ser Thr Ser Ala Ala Val Ala Leu Asp Ala Val Asn Tyr
                35 40 45
Ala Asp Asn His Pro Leu Trp Ile Gly Thr Val Asn Leu Glu Ala Gly
        50 55 60
Asp Val Val Glu Tyr Lys Tyr Ile Asn Val Gly Gln Asp Gly Ser Val
65 70 75 80
Thr Trp Glu Ser Asp Pro Asn His Thr Tyr Thr Val Pro Ala Val Ala
                                85 90 95
Cys Val Thr Gln Val Val Lys Glu Asp Thr Trp Gln Ser
                        100 105
SEQ ID NO: 12
SVDDFI: start of the TrGA mature protein
```

| | |
|---|---|
| <210> | 12 |
| <211> | 6 |
| <212> | PRT |
| <213> | Trichoderma reesei |
| <400> | 12 |

Ser Val Asp Asp Phe Ile
1 5
SEQ ID NO: 13:
Trichoderma reesei glucoamylase CS4 variant, mature protein; without
signal peptide

| | |
|---|---|
| <212> | PRT |
| <213> | Trichoderma reesei |
| 1 | SVDDFISTET PIALNNLLCN VGPDGCRAFG TSAGAVIASP STIDPDYYYM |
| 51 | WTRDSALVFK NLIDRFTETY DAGLQRRIEQ YITAQVTLQG LSNPSGSLAD |
| 101 | GSGLGEPKFE LTLKPFTGNW GRPQRDGPAL RAIALIGYSK WLINNNYQST |
| 151 | VSNVIWPIVR NDLNYVAQYW NQTGFDLWEE VNGSSFFTVA NQHRALVEGA |
| 201 | TLAATLGQSG SAYSSVAPQV LCFLQRFWVS SGGYVDSNIN TNEGRTGKDV |
| 251 | NSVLTSIHTF DPNLGCDAGT FQPCSDKALS NLKVVVDSFR SIYGVNKGIP |
| 301 | AGAAVAIGRY AEDVYYNGNP WYLATFAAAE QLYDAIYVWK KTGSITVTAT |
| 351 | SLAFFQELVP GVTAGTYSSS SSTFTNIINA VSTYADGFLS EAAKYVPADG |
| 401 | SLAEQFDRNS GTPLSAVHLT WSYASFLTAA ARRAGIVPPS WANSSASTIP |
| 451 | STCSGASVVG SYSRPTATSF PPSQTPKPGV PSGTPYTPLP CATPTSVAVT |
| 501 | FHELVSTQFG HTVKVAGNAA ALGNWSTSAA VALDAVNYRD NHPLWIGTVN |
| 551 | LEAGDVVEYK YIIVGQDGSV TWESDPNHTY TVPAVACVTQ VVKEDTWQS |

SEQ ID NO: 14:
Trichoderma reesei glucoamylase R_A_1 variant, mature protein; without
signal peptide

| | |
|---|---|
| <212> | PRT |
| <213> | Trichoderma reesei |
| 1 | SVDDFISTET PIALNNLLCN VGPDGCRAVG TSAGAVIASP STIDPDYYYM |
| 51 | WTRDSALVFK NLIDRFTETY DAGLQRRIEQ YITAQVTLQG LSNPSGSLAD |
| 101 | GSGLGEPKFE LTLKPFTGNW GRPQRDGPAL RAIALIGYSK WLINNNYQST |
| 151 | VSNVIWPIVR NDLNYVAQYW NQTGFDLWEE VNGSSFFTVA NQHRALVEGA |
| 201 | TLAATLGQSG SAYSSVAPQV LCFLQRFWVS SGGYVDSNIN TNEGRTGKDV |
| 251 | NSVLTSIHTF DPNLGCDAGT FQPCSDKALS NLKVVVDSFR SIYGVNKGIP |
| 301 | AGAAVAIGRY AEDVYYNGNP WYLATFAAAE QLYDAIYVWK KTGSITVTAT |
| 351 | SLAFFQELVP GVTAGTYSSS SSTFTNIINA VSTYADGFLS EAAKYVPADG |
| 401 | SLAEQFDRNS GTPLSAVHLT WSYASFLTAA ARRAGIVPPS WANSSASTIP |
| 451 | STCSGASVVG SYSRPTATSF PPSQTPKPGV PSSTPYTPLP CATPTSVAVT |
| 501 | FHELVSTQFG HTVKVAGNAA ALGNWSTSAA VALDAVNYRD NHPLWIGTVN |
| 551 | LEAGDVVEYK YIIVGQDGSV TWESDPNHTY TVPAVACVTQ VVKEDTWQS |

SEQ ID NO: 15:
Trichoderma reesei glucoamylase R_C_1 variant, mature protein; without
signal peptide

| | |
|---|---|
| <212> | PRT |
| <213> | Trichoderma reesei |
| 1 | SVDDFISTET PIALNNLLCN VGPDGCRAFG TSAGAVIASP STIDPDYVYM |
| 51 | WTRDSALVFK NLIDRFTETY DAGLQRRIEQ YITAQVTLQG LSNPSGSEAD |
| 101 | GSGLGEPKFE LTLKPFTGNW GRPQRDGPAL RAIALIGYSK WLINNNYQST |
| 151 | VSNVIWPIVR NDLNYVAQYW NQTGFDLWEE VNGSSFFTVA NQHRALVEGA |
| 201 | TLAATLGQSG SAYSSVAPQV LCFLQRFWVS SGGYVDSNIN TNEGRTGKDV |
| 251 | NSVLTSIHTF DPNLGCDAGT FQPCSDKALS NLKVVVDSFR SIYGVNKGIP |
| 301 | AGAAVAIGRY AEDVYYNGNP WYLATFAAAE QLYDAIYVWK KTGSITVTAT |
| 351 | SLAFFQELVP GVTAGTYSSS SSTFTNIINA VSTYADGFLS EAAKYVPADG |
| 401 | SLAEQFDRNS GTPLSAVHLT WSYASFLTAA ARRAGIVPPS WANSSASTIP |
| 451 | STCSGASVVG SYSRPTATSF PPSQTPKPGV PSGTPYTPLP CATPTSVAVT |
| 501 | FSELVSTQFG HTVKVAGNAA ALGNWSTSAA VALDAVNYRD NHPLWIGTVN |
| 551 | LEAGDVVEYK YIIVGQDGSV TWESDPNHTY TVPAVACVTQ VVKEDTWQS |

SEQ ID NO: 16:
Trichoderma reesei glucoamylase R_A_6 variant, mature protein; without
signal peptide

| | |
|---|---|
| <212> | PRT |
| <213> | Trichoderma reesei |
| 1 | SVDDFISTET PIALNNLLCN VGPDGCRAVG TSAGAVIASP STIDPDYYYM |
| 51 | WTRDSALVFK NLIDRFTETY DAGLQRRIEQ YITAQVTLQG LSNPSGMLAD |
| 101 | GSGLGEPKFE LTLKPFTGNW GRPQRDGPAL RAIALIGYSK WLINNNYQST |
| 151 | VSNVIWPIVR NDLNYVAQYW NQTGFDLWEE VNGSSFFTVA NQHRALVEGA |
| 201 | TLAATLGQSG SAYSSVAPQV LCFLQRFWVS SGGYVDSNIN TNEGRTGKDV |
| 251 | NSVLTSIHTF DPNLGCDAGT FQPCSDKALS NLKVVVDSFR SIYGVNKGIP |
| 301 | AGAAVAIGRY AEDVYYNGNP WYLATFAAAE QLYDAIYVWK KTGSITVTAT |
| 351 | SLAFFQELVP GVTAGTYSSS SSTFTNIINA VSTYADGFLS EAAKYVPADG |
| 401 | SLAEQFDRNS GTPLSALHLT WSYASFLTAT ARRAGIVPPS WANSSASTIP |
| 451 | STCSGASVVG SYSRPTATSF PPSQTPKPGV PSSWPYTPLP CATPTSVAVT |
| 501 | FHELVSTQFG QTVKVAGNAA ALGNWSTSAA VALDAVNYAD NHPLWIGTVN |
| 551 | LEAGDVVEYK YINVGQDGSV TWESDPNHTY TVPAVACVTQ VVKEDTWQS |

SEQ ID NO: 17:
Trichoderma reesei glucoamylase R_C_13 variant, mature protein; without
signal peptide

| | |
|---|---|
| <212> | PRT |
| <213> | Trichoderma reesei |
| 1 | SVDDFISTET PIALNNLLCN VGPDGCRAFG TSAGAVIASP STIDPDYYYM |
| 51 | WTRDSALVFK NLIDRFTETY DAGLQRRIEQ YITAQVTLQG LSNPSGSLAD |
| 101 | GSGLGEPKFE LTLKPFTGNW GRPQRDGPAL RAIALIGYSK WLINNNYQST |
| 151 | VSNVIWPIVR NDLNYVAQYW NQTGFDLWEE VNGSSFFTVA NQHRALVEGA |
| 201 | TLAATLGQSG SAYSSVAPQV LCFLQRFWVS SGGYVDSNIN TNEGRTGKDV |
| 251 | NSVLTSIHTF DPNLGCDAGT FQPCSDKALS NLKVVVDSFR SIYGVNKGIP |
| 301 | AGAAVAIGRY AEDVYYNGNP WYLATFAAAE QLYDAIYVWK KTGSITVTAT |
| 351 | SLAFFQELVP GVTAGTYSSS SSTFTNIINA VSTYADGFLS EAAKYVPADG |
| 401 | SLAEQFDRNS GTPLSAVHLT WSYASFLTAA ARRAGIVPPS WANSSASTIP |
| 451 | STCSGASVVG SYSRPTATSF PPSQTPKPGV PSGTPYTPLP CATPTSVAVT |
| 501 | FHELVSTQFG HTVKVAGNAA ALGNWSTSAA VALDAVNYRD NHPLWIGTVN |
| 551 | LEAGDVVEYK YIIVGQDGSV TWESDPNHTY TVPAVACVTQ VVKEDTWQS |

SEQ ID NO: 18:
Aspergillus awamori glucoamylase (AaGA), mature full-length, without
signal peptide

| | |
|---|---|
| <212> | PRT |
| <213> | Aspergillus awamori |
| 1 | TLDSWLSNEA TVARTAILNN IGADGAWVSG ADSGIVVASP STDNPDYFYT |
| 51 | WTRDSGLVIK TLVDLFRNGD TDLLSTIENY ISSQAIVQGI SNPSGDLSSG |
| 101 | GLGEPKFNVD ETAYTGSWGR PQRDGPALRA TAMIGFRQWL LDNGYTSAAT |
| 151 | EIVWPLVRND LSYVAQYWNQ TGYDLWEEVN GSSFFTIAVQ HRALVEGSAF |
| 201 | ATAVGSSCSW CDSQAPQILC YLQSFWTGEY ILANFDSSRS GKDTNTLLGS |
| 251 | IHTFDPEAGC DDSTFQPCSP RALANHKEVV DSFRSIYTLN DGLSDSEAVA |
| 301 | VGRYPKDSYY NGNPWFLCTL AAAEQLYDAL YQWDKQGSLE ITDVSLDFFQ |
| 351 | ALYSDAATGT YSSSSSTYSS IVDAVKTFAD GFVSIVETHA ASNGSLSEQY |
| 401 | DKSDGDELSA RDLTWSYAAL LTANNRRNSV MPPSWGETSA SSVPGTCAAT |
| 451 | SASGTYSSVT VTSSPSIVAT GGTTTTATTT GFGGVTSTSK TTTTASKTST |
| 501 | TTSSTSCTTP TAVAVTFDLT ATTTYGENIY LVGSISQLGD WDTSDGIALS |
| 551 | ADKYTSSNPL WYVTVTLPAG ESFEYKFIRI ESDDSVEWES DPNREYTVPQ |
| 601 | ACGESTATVT DTWR |

SEQ ID NO: 19:
Aspergillus niger glucoamylase (AnGA), mature full-length, without signal
peptide

| | |
|---|---|
| <212> | PRT |
| <213> | Aspergillus niger |
| 1 | ATLDSWLSNE ATVARTAILN NIGADGAWVS GADSGIVVAS PSTDNPDYFY |
| 51 | TWTRDSGLVL KTLVDLFRNG DTSLLSTIEN YISAQAIVQG ISNPSGDLSS |
| 101 | GAGLGEPKFN VDETAYTGSW GRPQRDGPAL RATAMIGFGQ WLLDNGYTST |
| 151 | ATDIVWPLVR NDLSYVAQYW NQTGYDLWEE VNGSSFFTIA VQHRALVEGS |
| 201 | AFATAVGSSC SWCDSQAPEI LCYLQSFWTG SFILANFDSS RSGKDANTLL |
| 251 | GSIHTFDPEA ACDDSTFQPC SPRALANHKE VVDSFRSIYT LNDGLSDSEA |
| 301 | VAVGRYPEDT YYNGNPWFLC TLAAAEQLYD ALYQWDKQGS LEVTDVSLDF |
| 351 | FKALYSDAAT GTYSSSSSTY SSIVDAVKTF ADGFVSIVET HAASNGSMSE |
| 401 | QYDKSDGEQL SARDLTWSYA ALLTANNRRN SVVPASWGET SASSVPGTCA |
| 451 | ATSAIGTYSS VTVTSWPSIV ATGGTTTTAT PTGSGSVTST SKTTATASKT |
| 501 | STSTSSTSCT TPTAVAVTFD LTATTTYGEN IYLVGSISQL GDWETSDGIA |
| 551 | LSADKYTSSD PLWYVTVTLP AGESFEYKFI RIESDDSVEW ESDPNREYTV |
| 601 | PQACGTSTAT VTDTWR |

SEQ ID NO: 20:
Aspergillus oryzae glucoamylase (AoGA), mature full-length, without
signal peptide

| | |
|---|---|
| <212> | PRT |
| <213> | Aspergillus oryzae |
| 1 | HPSFPIHKRQ SDLNAFIEAQ TPIAKQGVLN NIGADGKLVE GAAAGIVVAS |
| 51 | PSKSNPDYFY TWTRDAGLTM EEVIEQFIGG DATLESTIQN YVDSQANEQA |
| 101 | VSNPSGGLSD GSGLAEPKFY VNISQFTDSW GRPQRDGPAL RASALIAYGN |
| 151 | SLISSDKQSV VKANIWPIVQ NDLSYVGQYW NQTGFDLWEE VQGSSFFTVA |
| 201 | VQHKALVEGD AFAKALGEEC QACSVAPQIL CHLQDFWNGS AVLSNLPTNG |
| 251 | RSGLDTNSLL GSIHTFDPAA ACDDTTFQPC SSRALSNHKL VVDSFRSVYG |
| 301 | INNGRGAGKA AAVGPYAEDT YQGGNPWYLT TLVAAELLYD ALYQWDKQGQ |
| 351 | VNVTETSLPF FKDLSSNVTT GSYAKSSSAY ESLTSAVKTY ADGFISVVQE |
| 401 | YTPDGGALAE QYSRDQGTPV SASDLTWSYA AFLSAVGRRN GTVPASWGSS |
| 451 | TANAVPSQCS GGTVSGSYTT PTVGSW |

SEQ ID NO: 21:
Humicola grisea glucoamylase (HgGA), mature full-length, without signal
peptide

| | |
|---|---|
| <212> | PRT |
| <213> | Humicola grisea |
| 1 | RPHGSSRLQE RAAVDTFINT EKPIAWNKLL ANIGPNGKAA PGAAAGVVIA |
| 51 | SPSRTDPPYF FTWTPDAALV LTGIIESLGH NYNTTLQQVS NPSGTFADGS |
| 101 | GLGEAKFNVD LTAFTGEWGR PQRDGPPLRA IALIQYAKWL IANGYKSTAK |
| 151 | SVVWPVVKND LAYTAQYWNE TGFDLWEEVP GSSFFTIASS HRALTEGAYL |
| 201 | AAQLDTECPP CTTVAPQVLC FQQAFWNSKG NYVVSTSTAG EYRSGKDANS |
| 251 | ILASIHNFDP EAGCDNLTFQ PCSERALANH KAYVDSFRNL YAINKGIAQG |
| 301 | KAVAVGRYSE DVYYNGNPWY LANFAAAEQL YDAIYVWNKQ GSITVTSVSL |
| 351 | PFFRDLVSSV STGTYSKSSS TFTNIVNAVK AYADGFIEVA AKYTPSNGAL |
| 401 | AEQYDRNTGK PDSAADLTWS YSAFLSAIDR RAGLVPPSWR ASVAKSQLPS |
| 451 | TCSRIEVAGT YVAATSTSFP SKQTPNPSAA PSPSPYPTAC ADASEVYVTF |
| 501 | NERVSTAWGE TIKVVGNVPA LGNWDTSKAV TLSASGYKSN DPLWSITVPI |
| 551 | KATGSAVQYK YIKVGTNGKI TWESDPNRSI TLQTASSAGK CAAQTVNDSW |
| 601 | R |

SEQ ID NO: 22:
Hypocrea vinosa glucoamylase (HvGA), mature full-length, without signal peptide

| | |
|---|---|
| <212> | PRT |
| <213> | Hypocrea vinosa |
| 1 | RPGSNGLSDI TKRSVDSFIS AETPIALNNL LCNVGPDGCR AFGTSAGAVI |
| 51 | ASPSTVDPDY YYMWTRDSAL VFKNIVDRFT QKYDAGLQRR IEQYISAQVT |
| 101 | LQGISNPSGS LSDGSGLGEP KFELTLNQFT GNWGRPQRDG PALRAIALIG |
| 151 | YSKWLINNNY QSTVSSVIWP IVKNDLNYVA QYWNQTGFDL WEEVNGSSFF |
| 201 | TVANQHRALV EGATLATTLG QSGSTYSSVA PQILCFLQRF WVSGSYIDSN |
| 251 | INVNEGRTGK DANSLLASIH TFDPSLGCDA STFQPCSDKA LSNLKVVVDS |
| 301 | FRSIYGVNSG ISASSAVAIG RYPEDVYFNG NPWYLATFAA AEQLYDALYV |
| 351 | WKQAGSITVT STSLAFFQQL VPGVAAGTYS SSQSTYTSII NAVSAYADGF |
| 401 | MNEAAKYVPA DGSLAEQFDK NSGTPLSAVH LTWSYASFLT AADRRAGIVP |
| 451 | SSWASSGANT VPSSCSGASV VGSYSRPTAT SFPPSQTPKP GVPSGTPFTP |
| 501 | IPCATPTSVA VTFHELATTQ FGQTIKVVGS VPELGNWSTN AAVALNAYNY |
| 551 | ASNHPLWLGS INLAAGEVVQ YKYINVGSDG SVTWESDPNH TYTVPAVACV |
| 601 | TQVVKEDTWQ S |

SEQ ID NO: 23:
Talaromyces GA, mature protein

| | |
|---|---|
| <210> | 384 |
| <211> | 588 |
| <212> | PRT |
| <213> | Talaromyces sp. |

```
Gly Ser Leu Asp Ser Phe Leu Ala Thr Glu Thr Pro Ile Ala Leu Gln
1 5 10 15
Gly Val Leu Asn Asn Ile Gly Pro Asn Gly Ala Asp Val Ala Gly Ala
                        20 25 30
Ser Ala Gly Ile Val Val Ala Ser Pro Ser Arg Ser Asp Pro Asp Tyr
                35 40 45
Phe Tyr Ser Trp Thr Arg Asp Ala Ala Leu Thr Ala Lys Tyr Leu Val
        50 55 60
Asp Ala Phe Ile Ala Gly Asn Lys Asp Leu Glu Gln Thr Ile Gln Glu
65 70 75 80
Tyr Ile Ser Ala Gln Ala Gln Val Gln Thr Ile Ser Asn Pro Ser Gly
                                85 90 95
Asp Leu Ser Thr Gly Gly Leu Gly Glu Pro Lys Phe Asn Val Asn Glu
                        100 105 110
Thr Ala Phe Thr Gly Pro Trp Gly Arg Pro Gln Arg Asp Gly Pro Ala
                115 120 125
Leu Arg Ala Thr Ala Leu Ile Ala Tyr Ala Asn Tyr Leu Ile Asp Asn
        130 135 140
Gly Gln Ala Ser Thr Ala Asp Glu Ile Ile Trp Pro Ile Val Gln Asn
145 150 155 160
Asp Leu Ser Tyr Val Thr Gln Tyr Trp Asn Ser Ser Thr Phe Asp Leu
                                165 170 175
Trp Glu Glu Val Glu Gly Ser Ser Phe Phe Thr Thr Ala Val Gln His
                        180 185 190
Arg Ala Leu Val Glu Gly Asn Ala Leu Ala Thr Arg Leu Asn His Thr
                195 200 205
Cys Pro Asn Cys Val Ser Gln Ala Pro Gln Val Leu Cys Phe Leu Gln
        210 215 220
Ser Tyr Trp Thr Gly Ser Tyr Val Leu Ala Asn Phe Gly Gly Ser Gly
225 230 235 240
Arg Ser Gly Lys Asp Val Asn Ser Ile Leu Gly Ser Ile His Thr Phe
                                245 250 255
Asp Pro Ala Gly Gly Cys Asp Asp Ser Thr Phe Gln Pro Cys Ser Ala
                        260 265 270
Arg Ala Leu Ala Asn His Lys Val Val Thr Asp Ser Phe Arg Ser Val
                275 280 285
Tyr Ala Val Asn Ser Gly Ile Ala Glu Gly Ser Ala Val Ala Val Gly
        290 295 300
Arg Tyr Pro Glu Asp Val Tyr Gln Gly Gly Asn Pro Trp Tyr Leu Ala
305 310 315 320
Thr Ala Ala Ala Ala Glu Gln Leu Tyr Asp Ala Ile Tyr Gln Trp Asn
                                325 330 335
Lys Ile Gly Ser Ile Ser Ile Thr Asp Val Ser Leu Ala Phe Phe Gln
                        340 345 350
Asp Ile Tyr Pro Ser Ala Ala Val Gly Thr Tyr Asn Ser Gly Ser Ser
                355 360 365
Thr Phe Asn Asp Ile Ile Ser Ala Val Gln Thr Tyr Ala Asp Gly Tyr
        370 375 380
Leu Ser Ile Ile Glu Lys Tyr Thr Pro Ser Asp Gly Ser Leu Thr Glu
385 390 395 400
Gln Phe Ser Arg Ser Asp Gly Thr Pro Leu Ser Ala Ser Gly Leu Thr
                                405 410 415
Trp Ser Tyr Ala Ser Leu Leu Thr Ala Ala Ala Arg Arg Gln Ser Ile
                        420 425 430
Val Pro Ala Ser Trp Gly Glu Ser Ser Ala Ser Ser Val Pro Ala Val
                435 440 445
Cys Ser Ala Thr Ser Ala Thr Gly Pro Tyr Ser Thr Ala Thr Asn Thr
        450 455 460
Ala Trp Pro Ser Ser Gly Ser Gly Pro Ser Thr Thr Thr Ser Val Pro
465 470 475 480
Cys Thr Thr Pro Thr Ser Val Ala Val Thr Phe Asp Glu Ile Val Ser
                                485 490 495
Thr Thr Tyr Gly Glu Thr Ile Tyr Leu Ala Gly Ser Ile Pro Glu Leu
                        500 505 510
Gly Asn Trp Ser Pro Ser Ser Ala Ile Pro Leu Arg Ala Asp Ala Tyr
                515 520 525
Thr Ser Ser Asn Pro Leu Trp Tyr Val Thr Leu Asn Leu Pro Ala Gly
        530 535 540
Thr Ser Phe Glu Tyr Lys Phe Phe Lys Lys Glu Thr Asp Gly Thr Ile
545 550 555 560
Val Trp Glu Asp Asp Pro Asn Arg Ser Tyr Thr Val Pro Ala Tyr Cys
                                565 570 575
Gly Gln Thr Thr Ala Ile Leu Asp Asp Ser Trp Gln
                        580 585
SEQ ID NO: 24:
Humicola grisea GA, SBD
```

| | |
|---|---|
| <210> | 385 |
| <211> | 112 |
| <212> | PRT |
| <213> | Humicola grisea |

```
Cys Ala Asp Ala Ser Glu Val Tyr Val Thr Phe Asn Glu Arg Val Ser
1 5 10 15
Thr Ala Trp Gly Glu Thr Ile Lys Val Val Gly Asn Val Pro Ala Leu
                        20 25 30
Gly Asn Trp Asp Thr Ser Lys Ala Val Thr Leu Ser Ala Ser Gly Tyr
                35 40 45
Lys Ser Asn Asp Pro Leu Trp Ser Ile Thr Val Pro Ile Lys Ala Thr
        50 55 60
Gly Ser Ala Val Gln Tyr Lys Tyr Ile Lys Val Gly Thr Asn Gly Lys
65 70 75 80
Ile Thr Trp Glu Ser Asp Pro Asn Arg Ser Ile Thr Leu Gln Thr Ala
                                85 90 95
Ser Ser Ala Gly Lys Cys Ala Ala Gln Thr Val Asn Asp Ser Trp Arg
                        100 105 110
SEQ ID NO: 25:
Thermomyces lanuginosus GA, SBD
```

| | |
|---|---|
| <210> | 386 |
| <211> | 109 |
| <212> | PRT |
| <213> | Thermomyces lanuginosus |

```
Cys Thr Pro Pro Ser Glu Val Thr Leu Thr Phe Asn Ala Leu Val Asp
1 5 10 15
Thr Ala Phe Gly Gln Asn Ile Tyr Leu Val Gly Ser Ile Pro Glu Leu
                          20 25 30
Gly Ser Trp Asp Pro Ala Asn Ala Leu Leu Met Ser Ala Lys Ser Trp
                35 40 45
Thr Ser Gly Asn Pro Val Trp Thr Leu Ser Ile Ser Leu Pro Ala Gly
        50 55 60
Thr Ser Phe Glu Tyr Lys Phe Ile Arg Lys Asp Asp Gly Ser Ser Asp
65 70 75 80
Val Val Trp Glu Ser Asp Pro Asn Arg Ser Tyr Asn Val Pro Lys Asp
                                85 90 95
Cys Gly Ala Asn Thr Ala Thr Val Asn Ser Trp Trp Arg
                        100 105
SEQ ID NO: 26:
Talaromyces emersonii GA, SBD
```

| | |
|---|---|
| <210> | 387 |
| <211> | 108 |
| <212> | PRT |
| <213> | Talaromyces emersonii |

```
Cys Thr Thr Pro Thr Ser Val Ala Val Thr Phe Asp Glu Ile Val Ser
1 5 10 15
Thr Ser Tyr Gly Glu Thr Ile Tyr Leu Ala Gly Ser Ile Pro Glu Leu
                        20 25 30
Gly Asn Trp Ser Thr Ala Ser Ala Ile Pro Leu Arg Ala Asp Ala Tyr
                35 40 45
Thr Asn Ser Asn Pro Leu Trp Tyr Val Thr Val Asn Leu Pro Pro Gly
        50 55 60
Thr Ser Phe Glu Tyr Lys Phe Phe Lys Asn Gln Thr Asp Gly Thr Ile
65 70 75 80
Val Trp Glu Asp Asp Pro Asn Arg Ser Tyr Thr Val Pro Ala Tyr Cys
                                85 90 95
Gly Gln Thr Thr Ala Ile Leu Asp Asp Ser Trp Gln
                        100 105
SEQ ID NO: 27:
Aspergillus niger GA, SBD
```

| | |
|---|---|
| <210> | 388 |
| <211> | 108 |
| <212> | PRT |
| <213> | Aspergillus niger |

```
Cys Thr Thr Pro Thr Ala Val Ala Val Thr Phe Asp Leu Thr Ala Thr
1 5 10 15
Thr Thr Tyr Gly Glu Asn Ile Tyr Leu Val Gly Ser Ile Ser Gln Leu
                        20 25 30
Gly Asp Trp Glu Thr Ser Asp Gly Ile Ala Leu Ser Ala Asp Lys Tyr
                35 40 45
Thr Ser Ser Asp Pro Leu Trp Tyr Val Thr Val Thr Leu Pro Ala Gly
        50 55 60
Glu Ser Phe Glu Tyr Lys Phe Ile Arg Ile Glu Ser Asp Asp Ser Val
65 70 75 80
Glu Trp Glu Ser Asp Pro Asn Arg Glu Tyr Thr Val Pro Gln Ala Cys
                                85 90 95
Gly Thr Ser Thr Ala Thr Val Thr Asp Thr Trp Arg
                        100 105
SEQ ID NO: 28:
Aspergillus awamori GA, SBD
```

| | |
|---|---|
| <210> | 389 |
| <211> | 108 |
| <212> | PRT |
| <213> | Aspergillus awamori |

```
Cys Thr Thr Pro Thr Ala Val Ala Val Thr Phe Asp Leu Thr Ala Thr
1 5 10 15
Thr Thr Tyr Gly Glu Asn Ile Tyr Leu Val Gly Ser Ile Ser Gln Leu
                        20 25 30
Gly Asp Trp Asp Thr Ser Asp Gly Ile Ala Leu Ser Ala Asp Lys Tyr
                35 40 45
Thr Ser Ser Asn Pro Leu Trp Tyr Val Thr Val Thr Leu Pro Ala Gly
        50 55 60
Glu Ser Phe Glu Tyr Lys Phe Ile Arg Ile Glu Ser Asp Asp Ser Val
65 70 75 80
Glu Trp Glu Ser Asp Pro Asn Arg Glu Tyr Thr Val Pro Gln Ala Cys
                                85 90 95
Gly Glu Ser Thr Ala Thr Val Thr Asp Thr Trp Arg
                        100 105
SEQ ID NO: 29:
Thielavia terrestris GA, SBD
```

| | |
|---|---|
| <210> | 390 |
| <211> | 108 |
| <212> | PRT |
| <213> | Thielavia terrestris |
| <400> | 390 |

```
Cys Ser Thr Pro Thr Ala Val Ala Val Thr Phe Asn Glu Arg Val Thr
1 5 10 15
Thr Gln Trp Gly Gln Thr Ile Lys Val Val Gly Asp Ala Ala Ala Leu
                        20 25 30
Gly Gly Trp Asp Thr Ser Lys Ala Val Pro Leu Ser Ala Ala Gly Tyr
                35 40 45
Thr Ala Ser Asp Pro Leu Trp Ser Gly Thr Val Asp Leu Pro Ala Gly
        50 55 60
Leu Ala Val Gln Tyr Lys Tyr Ile Asn Val Ala Ala Asp Gly Gly Val
65 70 75 80
Thr Trp Glu Ala Asp Pro Asn His Ser Phe Thr Val Pro Ala Ala Cys
                                85 90 95
Gly Thr Thr Ala Val Thr Arg Asp Asp Thr Trp Gln
                        100 105
SEQ ID NO: 30:
Trichoderma reesei wt glucoamylase optimized cDNA (2535 bp-
4433 bp, direct) 1899 bp (pEntry-GA WT)
ATGCACGTCCTGTCGACTGCGGTGCTGCTCGGCTCCGTTGCCGTTCAAAAGGTCCTGGGAAGACCAGGATCAAGC
GGTCTGTCCGACGTCACCAAGAGGTCTGTTGACGACTTCATCAGCACCGAGACGCCTATTGCACTGAACAATCTT
CTTTGCAATGTTGGTCCTGATGGATGCCGTGCATTCGGCACATCAGCTGGTGCGGTGATTGCATCTCCCAGCACA
ATTGACCCGGACTACTATTACATGTGGACGCGAGATAGCGCTCTTGTCTTCAAGAACCTCATCGACCGCTTCACC
GAAACGTACGATGCGGGCCTGCAGCGCCGCATCGAGCAGTACATTACTGCCCAGGTCACTCTCCAGGGCCTCTCT
AACCCCTCGGGCTCCCTCGCGGACGGCTCTGGTCTCGGCGAGCCCAAGTTTGAGTTGACCCTGAAGCCTTTCACC
GGCAACTGGGGTCGACCGCAGCGGGATGGCCCAGCTCTGCGAGCCATTGCCTTGATTGGATACTCAAAGTGGCTC
ATCAACAACAACTATCAGTCGACTGTGTCCAACGTCATCTGGCCTATTGTGCGCAACGACCTCAACTATGTTGCC
CAGTACTGGAACCAAACCGGCTTTGACCTCTGGGAAGAAGTCAATGGGAGCTCATTCTTTACTGTTGCCAACCAG
CACCGAGCACTTGTCGAGGGCGCCACTCTTGCTGCCACTCTTGGCCAGTCGGGAAGCGCTTATTCATCTGTTGCT
CCCCAGGTTTTGTGCTTTCTCCAACGATTCTGGGTGTCGTCTGGTGGATACGTCGACTCCAACATCAACACCAAC
GAGGGCAGGACTGGCAAGGATGTCAACTCCGTCCTGACTTCCATCCACACCTTCGATCCCAACCTTGGCTGTGAC
GCAGGCACCTTCCAGCCATGCAGTGACAAAGCGCTCTCCAACCTCAAGGTTGTTGTCGACTCCTTCCGCTCCATC
TACGGCGTGAACAAGGGCATTCCTGCCGGTGCTGCCGTCGCCATTGGCCGGTATGCAGAGGATGTGTACTACAAC
GGCAACCCTTGGTATCTTGCTACATTTGCTGCTGCCGAGCAGCTGTACGATGCCATCTACGTCTGGAAGAAGACG
GGCTCCATCACGGTGACCGCCACCTCCCTGGCCTTCTTCCAGGAGCTTGTTCCTGGCGTGACGGCCGGGACCTAC
TCCAGCAGCTCTTCGACCTTTACCAACATCATCAACGCCGTCTCGACATACGCCGATGGCTTCCTCAGCGAGGCT
GCCAAGTACGTCCCCGCCGACGGTTCGCTGGCCGAGCAGTTTGACCGCAACAGCGGCACTCCGCTGTCTGCGCTT
CACCTGACGTGGTCGTACGCCTCGTTCTTGACAGCCACGGCCCGTCGGGCTGGCATCGTGCCCCCCTCGTGGGCC
AACAGCAGCGCTAGCACGATCCCCTCGACGTGCTCCGGCGCGTCCGTGGTCGGATCCTACTCGCGTCCCACCGCC
ACGTCATTCCCTCCGTCGCAGACGCCCAAGCCTGGCGTGCCTTCCGGTACTCCCTACACGCCCCTGCCCTGCGCG
ACCCCAACCTCCGTGGCCGTCACCTTCCACGAGCTCGTGTCGACACAGTTTGGCCAGACGGTCAAGGTGGCGGGC
AACGCCGCGGCCCTGGGCAACTGGAGCACGAGCGCCGCCGTGGCTCTGGACGCCGTCAACTATGCCGATAACCAC
CCCCTGTGGATTGGGACGGTCAACCTCGAGGCTGGAGACGTCGTGGAGTACAAGTACATCAATGTGGGCCAAGAT
GGCTCCGTGACCTGGGAGAGTGATCCCAACCACACTTACACGGTTCCTGCGGTGGCTTGTGTGACGCAGGTTGTC
AAGGAGGACACCTGGCAGTCGTAA
SEQ ID NO: 31:
Trichoderma reesei CS4 variant glucoamylase optimized cDNA
(2535 bp-4433 bp, direct) 1899 bp (pEntry-GA CS4)
ATGCACGTCCTGTCGACTGCGGTGCTGCTCGGCTCCGTTGCCGTTCAAAAGGTCCTGGGAAGACCAGGATCAAGC
GGTCTGTCCGACGTCACCAAGAGGTCTGTTGACGACTTCATCAGCACCGAGACGCCTATTGCACTGAACAATCTT
CTTTGCAATGTTGGTCCTGATGGATGCCGTGCATTCGGCACATCAGCTGGTGCGGTGATTGCATCTCCCAGCACA
ATTGACCCGGACTACTATTACATGTGGACGCGAGATAGCGCTCTTGTCTTCAAGAACCTCATCGACCGCTTCACC
GAAACGTACGATGCGGGCCTGCAGCGCCGCATCGAGCAGTACATTACTGCCCAGGTCACTCTCCAGGGCCTCTCT
AACCCCTCGGGCTCCCTCGCGGACGGCTCTGGTCTCGGCGAGCCCAAGTTTGAGTTGACCCTGAAGCCTTTCACC
GGCAACTGGGGTCGACCGCAGCGGGATGGCCCAGCTCTGCGAGCCATTGCCTTGATTGGATACTCAAAGTGGCTC
ATCAACAACAACTATCAGTCGACTGTGTCCAACGTCATCTGGCCTATTGTGCGCAACGACCTCAACTATGTTGCC
CAGTACTGGAACCAAACCGGCTTTGACCTCTGGGAAGAAGTCAATGGGAGCTCATTCTTTACTGTTGCCAACCAG
CACCGAGCACTTGTCGAGGGCGCCACTCTTGCTGCCACTCTTGGCCAGTCGGGAAGCGCTTATTCATCTGTTGCT
CCCCAGGTTTTGTGCTTTCTCCAACGATTCTGGGTGTCGTCTGGTGGATACGTCGACTCCAACATCAACACCAAC
GAGGGCAGGACTGGCAAGGATGTCAACTCCGTCCTGACTTCCATCCACACCTTCGATCCCAACCTTGGCTGTGAC
GCAGGCACCTTCCAGCCATGCAGTGACAAAGCGCTCTCCAACCTCAAGGTTGTTGTCGACTCCTTCCGCTCCATC
TACGGCGTGAACAAGGGCATTCCTGCCGGTGCTGCCGTCGCCATTGGCCGGTATGCAGAGGATGTGTACTACAAC
GGCAACCCTTGGTATCTTGCTACATTTGCTGCTGCCGAGCAGCTGTACGATGCCATCTACGTCTGGAAGAAGACG
GGCTCCATCACGGTGACCGCCACCTCCCTGGCCTTCTTCCAGGAGCTTGTTCCTGGCGTGACGGCCGGGACCTAC
TCCAGCAGCTCTTCGACCTTTACCAACATCATCAACGCCGTCTCGACATACGCCGATGGCTTCCTCAGCGAGGCT
GCCAAGTACGTCCCCGCCGACGGTTCGCTGGCCGAGCAGTTTGACCGCAACAGCGGCACTCCGCTGTCTGCGGTT
CACCTGACGTGGTCGTACGCCTCGTTCTTGACAGCCGCGGCCCGTCGGGCTGGCATCGTGCCCCCCTCGTGGGCC
AACAGCAGCGCTAGCACGATCCCCTCGACGTGCTCCGGCGCGTCCGTGGTCGGATCCTACTCGCGTCCCACCGCC
ACGTCATTCCCTCCGTCGCAGACGCCCAAGCCTGGCGTGCCTTCCGGTACTCCCTACACGCCCCTGCCCTGCGCG
ACCCCAACCTCCGTGGCCGTCACCTTCCACGAGCTCGTGTCGACACAGTTTGGCCATACGGTCAAGGTGGCGGGC
AACGCCGCGGCCCTGGGCAACTGGAGCACGAGCGCCGCCGTGGCTCTGGACGCCGTCAACTATCGTGATAACCAC
CCCCTGTGGATTGGGACGGTCAACCTCGAGGCTGGAGACGTCGTGGAGTACAAGTACATCATTGTGGGCCAAGAT
GGCTCCGTGACCTGGGAGAGTGATCCCAACCACACTTACACGGTTCCTGCGGTGGCTTGTGTGACGCAGGTTGTC
AAGGAGGACACCTGGCAGTCGTAA
```

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Various modifications and variations of the described embodiments will be apparent to those skilled in the art without departing from the scope and spirit of those embodiments. It should be understood that the subject matters as claimed should not be unduly limited to such specific embodiments. Indeed, various modifications of the described modes for carrying out the embodiments that are obvious to those skilled in the art are intended to be within the scope of the following claims.

1. A glucoamylase variant comprising one or two amino acid substitutions in the group of interface amino acids consisting of residues 502, 29, 43, 48, and 116 of SEQ ID NO: 2, or an equivalent position in a parent glucoamylase; and one, two or three amino acid substitutions in the group of catalytic core amino acid residues consisting of residues 98, 97, 147, 175, 483 and 484 of SEQ ID NO: 2, or an equivalent position in a parent glucoamylase, wherein the glucoamylase variant has at least 80% sequence identity with SEQ ID NO: 1, 2, 13, 18, 19, 20, 21, or 22.

2. The glucoamylase variant according to claim 1 comprising
a) an amino acid substitution at the residue corresponding to position 502 of SEQ ID NO: 2, or an equivalent position in a parent glucoamylase, and optionally an amino acid substitution selected from the group of interface amino acids consisting of residues 29, 43, 48, and 116 of SEQ ID NO: 2, or an equivalent position in a parent glucoamylase;
b) an amino acid substitution at the residue corresponding to position 98 of SEQ ID NO: 2, or an equivalent position in a parent glucoamylase, and optionally one or two amino acid substitutions selected from the group of catalytic core amino acid residues consisting of residues 97, 147, 175, 483 and 484 of SEQ ID NO: 2, or an equivalent position in a parent glucoamylase;
which glucoamylase variant at least has one amino acid substitution selected from said group of interface amino acids or said group of catalytic core amino acid residues;
wherein the glucoamylase variant has at least 80% sequence identity with SEQ ID NO: 1, 2, 13, 18, 19, 20, 21, or 22.

3. The glucoamylase variant according to claim 2 comprising
a) an amino acid substitution at the residue corresponding to position 502 of SEQ ID NO: 2, or an equivalent position in a parent glucoamylase,
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase, or an amino acid substitution at the residue corresponding to position 147 of SEQ ID NO: 2 or an equivalent position in a parent glucoamylase;
wherein the glucoamylase variant has at least 80% sequence identity with SEQ ID NO: 1, 2, 13, 18, 19, 20, 21, or 22.

4. The glucoamylase variant according to claim 3 comprising
a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 147 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

5. The glucoamylase variant according to claim 4 comprising
a) an amino acid substitution at the residue corresponding to position 502 of SEQ ID NO: 2, or an equivalent position in a parent glucoamylase;
b) an amino acid substitution at the residue corresponding to position 98 of SEQ ID NO: 2, or an equivalent position in a parent glucoamylase; and
c) an amino acid substitution at the residue corresponding to position 48 of SEQ ID NO: 2, or an equivalent position in a parent glucoamylase;
wherein the glucoamylase variant has at least 80% sequence identity with SEQ ID NO: 1, 2, 13, 18, 19, 20, 21, or 22.

6. The glucoamylase variant according to claim 5, comprising the following amino acid substitution H502S of SEQ ID NO:2, or an equivalent position in a parent glucoamylase.

7. The glucoamylase variant according to claim 6, comprising the following amino acid substitution L98E of SEQ ID NO:2, or an equivalent position in a parent glucoamylase.

8. The glucoamylase variant according to claim 7, comprising the following amino acid substitution Y48V of SEQ ID NO:2, or an equivalent position in a parent glucoamylase.

9. The glucoamylase variant according to claim 8, comprising the following amino acid substitution Y147R of SEQ ID NO:2, or an equivalent position in a parent glucoamylase.

10. The glucoamylase variant according to claim 9, comprising the amino acid substitution H502S of SEQ ID NO: 2 or 13; the amino acid substitution L98E of SEQ ID NO: 2 or 13; and the amino acid substitution Y48V of SEQ ID NO: 2 or 13, or the amino acid substitution Y147R of SEQ ID NO: 2 or 13; wherein the glucoamylase variant has at least 80% sequence identity with SEQ ID NO: 2 or 13.

11. The glucoamylase variant according to claim 10, wherein the parent glucoamylase is SEQ ID NO: 1, 2, 13, 18, 19, 20, 21, or 22.

12. The glucoamylase variant according to claim 11, wherein the parent glucoamylase is SEQ ID NO: 2 or 13.

13. The glucoamylase variant according to claim 12 further comprising one or two amino acid substitutions in the group of interface amino acids consisting of residues 24, 26, 27, 30, 40, 42, 44, 46, 49, 110, 111, 112, 114, 117, 118, 119, 500, 504, 534, 536, 537, 539, 541, 542, 543, 544, 546, 547, 548, 580, 583, 585, 587, 588, 589, 590, 591, 592, 594, and 596 of SEQ ID NO:2 or an equivalent position in a parent glucoamylase.

14. The glucoamylase variant according to claim 13 further comprising one, two or three amino acid substitutions in the group of catalytic core amino acids consisting of residues in positions 1 to 484 with exception of position 24, 26, 27, 29, 30, 40, 42, 43, 44, 46, 48, 49, 97, 98, 110, 111, 112, 114, 116, 117, 118, 119, 147, 175, 483 and 484 of SEQ ID NO: 2, or an equivalent position in a parent glucoamylase.

15. The glucoamylase variant according to claim 14, wherein the glucoamylase variant exhibits a RDF of at least 74.5%.

16. The glucoamylase variant according to claim 15, wherein the glucoamylase variant has at least 85% sequence identity with SEQ ID NO: 1, 2, 13, 18, 19, 20, 21, or 22.

17. The glucoamylase variant according to claim 16, wherein the glucoamylase variant has at least 80% sequence identity, such as at least 85%, 90%, 95%, or 99.5% sequence identity with SEQ ID NO: 2, or 13.

18. The glucoamylase variant according to claim 17, wherein the glucoamylase variant exhibits decreased thermostability as compared to the parent glucoamylase.

19. The glucoamylase variant according to claim 18, which glucoamylase variant is inactivated by pasteurisation such as using less than 16.8, 16, 15, 14, 13, 12, 11, 10, 9, 8, 7, 6, 5, or 4 pasteurisation units (PU) in beer.

20. The glucoamylase variant according to claim 19 consisting of SEQ ID NO: 14, 15 or 17.

21. A method for producing a glucoamylase variant as defined in any one of claim 1, the method comprising the steps of inducing synthesis of the glucoamylase variant in a host cell having heterologous expression of said glucoamylase variant, and optionally purifying the glucoamylase variant.

22. A composition comprising one or more glucoamylase variant(s) as defined in claim 1 such as an alcohol fermentation enzymatic composition, which composition optionally comprises one or more further enzyme(s) selected among alpha-amylase, beta-amylase, peptidase (for example protease, proteinase, endopeptidase, exopeptidase), pullulanase, isoamylase, cellulase, endo-glucanase and related beta-glucan hydrolytic accessory enzymes, xylanase and xylanase accessory enzymes (for example, arabinofuranosidase, ferulic acid esterase, xylan acetyl esterase), acetolactate decarboxylase and glucoamylase, including any combination(s) thereof.

23. Use of a glucoamylase variant as defined in claim 1 or a composition as defined in claim 22 in a fermentation, wherein said glucoamylase variant or composition is added before or during a fermentation step, wherein said fermentation step is optionally followed by a pasteurisation step, such as wherein said fermentation is comprised in a process for making a fermented beverage.

24. A method which comprises adding a glucoamylase variant as defined in claim 1 or a composition as defined in claim 22 before or during a fermentation step optionally followed by a pasteurisation step.

25. A method for production of a fermented beverage which comprises the following steps:
a) preparing a mash,
b) filtering the mash to obtain a wort, and
c) fermenting the wort to obtain a fermented beverage,
wherein a glucoamylase variant as defined in claim 1 or a composition as defined in claim 22 is added to:
i. the mash of step (a) and/or
ii. the wort of step (b) and/or
iii. the wort of step (c).
